(19) United States
(12) Patent Application Publication
(10) Pub. No.: US 2010/0003675 A1
(43) Pub. Date: Jan. 7, 2010

(54) Qtls for Mastitis Resistance in Cattle

(75) Inventors: Mogens Sando Lund, Orum (DK); Bente Flugel Majgren, Hobro (DK); Goutam Sahana, Tjele (DK); Ayman Mahmoud Sabry, Cairo (EG); Peter Sorensen, Viborg (DK); Soren Svendsen, Randers (DK); Bo Thomsen, Arhus (DK); Ingrid Lena Anderson-Eklund, Storvreta (SE); Helmi Johanna Vilkki, Jokioinen (FI); Christian Bendixen, Ulstrup (DK); Terhi Katariina Iso-Touru, Turku (FI); Sirja Maria Viitala, Tammela (FI); Nina Frederika Schulman, Ypaja (FI); Nicola Hastings, Midlothian (GB); John Lewis William Williams, Lodi (IT); John Arthur Woolliams, Peebles (GB); Ana Isabel Fernandez Avila, Madrid (ES); Haldja Viinalass, Tartu Maakond (EE); Sirje Varv, Tartu (EE)

Correspondence Address: WEINGARTEN, SCHURGIN, GAGNEBIN & LEBOVICI LLP, TEN POST OFFICE SQUARE, BOSTON, MA, 02109, US

(73) Assignees: MTT Agrifood Research Finland, Jokioinen (FI); Aarhus Universitet, Arthus (DK); Estonian University of Life Sciences, Tartu (EE)

(21) Appl. No.: 12/223,689

(22) PCT Filed: Feb. 5, 2007
(86) PCT No.: PCT/DK2007/000058

§ 371 Date: Sep. 11, 2009

(30) Foreign Application Priority Data
Feb. 6, 2006 (DK) PA 2006 00164
Jan. 30, 2007 (DK) PA 2007 00147

(51) Int. Cl. C12Q 1/68

(52) U.S. Cl. 435/6

(57) ABSTRACT

The invention relates to a method for determining mastitis resistance in bovine subjects, wherein mastitis resistance comprise resistance to both sub-clinical and clinical mastitis. In particular, the method of the invention involves identification of genetic markers and/or Quantitative Trait Locus (QTL) for the determination of mastitis resistance in a bovine subject. The determination of mastitis resistance involves resolution of the specific microsatellite status. Furthermore, the invention relates to a diagnostic kit for detection of genetic marker(s) associated with mastitis resistance. The method and kit of the present invention can be applied for selection of bovine subjects for breeding purposes. Thus, the invention provides a method of genetically selecting bovine subjects with mastitis resistance, thereby yielding cows less prone to mastitis.

### FIELD OF INVENTION

The present invention relates to a method for determining the resistance to mastitis in a bovine subject comprising detecting at least one genetic marker located on the bovine chromosomes BTA9 and BTA11. Furthermore, the present invention relates to a diagnostic kit for detecting the presence or absence of at least one genetic marker associated with resistance to mastitis.

### BACKGROUND OF INVENTION

Mastitis is the inflammation of the mammary gland or udder of the cow resulting from infection or trauma and mastitis is believed to be the most economically important disease in cattle.

The disease may be caused by a variety of agents. The primary cause of mastitis is the invasion of the mammary gland via the teat end by microorganisms.

Mastitis may be clinical or sub-clinical, with sub-clinical infection preceding clinical manifestations. Clinical mastitis (CM) can be detected visually through observing red and swollen mammary glands i.e. red swollen udder, and through the production of clotted milk. Once detected, the milk from mastitic cows is kept separate from the vat so that it will not affect the overall milk quality.

Sub-clinical mastitis is a type of mastitis characterized by high somatic cell counts (SCS), a normal or elevated body temperature, and milk samples that should test positive on culture. Thus, sub-clinical mastitis cannot be detected visually by swelling of the udder or by observation of the gland or the milk produced. Because of this, farmers do not have the option of diverting milk from sub-clinical mastitic cows. However, this milk is of poorer quality than that from non-infected cows and can thus contaminate the rest of the milk in the vat.

Mastitis can be detected by the use of somatic cell counts (SCS) in which a sample of milk from a cow is analysed for the presence of somatic cells (white blood cells). Somatic cells are part of the cow's natural defence mechanism and cell counts rise when the udder becomes infected. The number of somatic cells in a milk sample can be estimated indirectly by rolling-ball viscometer and Coulter counter.

As mastitis results in reduced quantity and quality of milk and products from milk, mastitis results in economic losses to the farmer and dairy industry. Therefore, the ability to determine the genetic basis of resistance to mastitis in a bovine is of immense economic significance to the dairy industry both in terms of daily milk production but also in breeding management, selecting for bovine subjects with resistance to mastitis. A method of genetically selecting bovine subjects with improved resistance that will yield cows less prone to mastitis would be desirable.

One approach to identify genetic determinants for genetic traits is the use of linkage disequilibrium (LD) mapping which aims at exploiting historical recombinants and has been shown in some livestock populations, including dairy cattle, to extend over very long chromosome segments when compared to human populations (Farnir et al., 2000). Once mapped, a Quantitative Trait Locus (QTL) can be usefully applied in marker assisted selection.

### Linkage Disequilibrium

Linkage disequilibrium reflects recombination events dating back in history and the use of LD mapping within families increases the resolution of mapping. LD exists when observed haplotypes in a population do not agree with the haplotype frequencies predicted by multiplying together the frequency of individual genetic markers in each haplotype. In this respect the term haplotype means a set of closely linked genetic markers present on one chromosome which tend to be inherited together. In order for LD mapping to be efficient the density of genetic markers needs to be compatible with the distance across which LD extends in the given population. In a study of LD in dairy cattle population using a high number of genetic markers (284 autosomal microsatellite markers) it was demonstrated that LD extends over several tens of centimorgans for intrachromosomal markers (Farnir et al. 2000). Similarly, Georges, M (2000) reported that the location of a genetic marker that is linked to a particular phenotype in livestock typically has a confidence interval of 20-30 cM (corresponding to maybe 500-1000 genes) (Georges, M., 2000). The existence of linkage disequilibrium is taken into account in order to use maps of particular regions of interest with high confidence.

In the present invention quantitative trait loci associated to clinical mastitis and/or SCS have been identified on bovine chromosome BTA9 which allows for a method for determining whether a bovine subject will be resistant to mastitis.

### SUMMARY OF INVENTION

It is of significant economic interest within the cattle industry to be able to select bovine subjects with increased resistance to mastitis and thereby avoid economic losses in connection with animals suffering from mastitis. The genetic predisposition for resistance to mastitis may be detected by the present invention. The present invention offers a method for determining the resistance to mastitis in a bovine subject based on genetic markers which are associated with and/or linked to resistance to mastitis.

Thus, one aspect of the present invention relates to a method for determining the resistance to mastitis in a bovine subject, comprising detecting in a sample from said bovine subject the presence or absence of at least one genetic marker that is linked to at least one trait indicative of mastitis resistance, wherein said at least one genetic marker is located on the bovine chromosome BTA9 in the region flanked by and including the polymorphic microsatellite markers C6orf93 and inra084 and/or BTA11 in the region flanked by and including the polymorphic microsatellite markers HELMTT43 and BM3501, wherein the presence or absence of said at least one genetic marker is indicative of mastitis resistance of said bovine subject or off-spring therefrom.

A second aspect of the present invention relates to a diagnostic kit for use in detecting the presence or absence in a bovine subject of at least one genetic marker associated with resistance to mastitis, comprising at least one oligonucleotide sequence and combinations thereof, wherein the nucleotide sequences are selected from any of SEQ ID NO.: 1 to SEQ ID NO.: 192 and/or any combination thereof.

### DESCRIPTION OF DRAWINGS

FIG. 1: Genome scan of BTA9 in relation to mastitis resistance characteristic of Danish Red families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIG. 2: Genome scan of BTA9 in relation to somatic cell count characteristic of Danish Red families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIG. 3: Genome scan of BTA9 in relation to mastitis resistance characteristic of Finnish Ayrshire families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIG. 4: Genome scan of BTA9 in relation to somatic cell count characteristic of Finnish Ayrshire families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIGS. 5a and 5b: Genome scan of BTA9 in relation to mastitis resistance characteristic of Swedish Red and White families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIGS. 6a and 6b: Genome scan of BTA9 in relation to somatic cell count characteristic of Swedish Red and White families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIG. 7: Genome scan of BTA9 in relation to mastitis resistance characteristic of Danish Holstein families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIG. 8: Genome scan of BTA9 in relation to somatic cell count characteristic of Danish Holstein families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIG. 9: QTL profile for the trait mastitis resistance showing LDLA/LD peak between the markers BMS2819 and INRA144 at 74.08 cM in Danish Red cattle. The X-axis represents the distance of the chromosome expressed in Morgan according to the positions employed in this analysis. The Y-axis represents the likelihood ratio test-statistics of the QTL analysis.

FIG. 10: QTL profile for the trait Somatic Cell Count showing LDLA peak between the markers BMS2819 and INRA144 at 74.075 cM in Danish Red cattle. The X-axis represents the distance of the chromosome expressed in Morgan according to the positions employed in this analysis. The Y-axis represents the likelihood ratio test-statistics of the QTL analysis.

FIG. 11: QTL profile for the trait mastitis resistance showing LDLA peak between the markers BM4208 and INRA144 in Finnish Ayrshire breed. The X-axis represents the distance of the chromosome expressed in Morgan according to the positions employed in this analysis. The Y-axis represents the likelihood ratio test-statistics of the QTL analysis.

FIG. 12: QTL profile for the trait mastitis resistance showing LDLA/LD peak between the markers BMS2819 and INRA144 in combined Finnish Ayrshire and Danish Red cattle. The X-axis represents the distance of the chromosome expressed in Morgan according to the positions employed in this analysis. The Y-axis represents the likelihood ratio test-statistics of the QTL analysis.

FIG. 13: QTL profile for the trait mastitis resistance showing LA peak at 60-80 cM markers BMS2819 and INRA144 in Swedish Red and White cattle. The X-axis represents the distance of the chromosome expressed in Morgan according to the positions employed in this analysis. The Y-axis represents the likelihood ratio test-statistics of the QTL analysis.

FIG. 14: QTL profile for SCS in Swedish Red and White cattle with LDLA evidence between markers BMS2819 and INRA084. The X-axis represents the distance of the chromosome expressed in Morgan according to the positions employed in this analysis. The Y-axis represents the likelihood ratio test-statistics of the QTL analysis.

FIG. 15: QTL profile for the trait mastitis resistance showing LDLA/LD peak between the markers BM4208 and INRA144 in combined analyses of Finnish Ayrshire, Danish Red cattle and Swedish Red and White cattle. The X-axis represents the distance of the chromosome expressed in Morgan according to the positions employed in this analysis. The Y-axis represents the likelihood ratio test-statistics of the QTL analysis.

FIG. 16: QTL profile for the trait mastitis resistance Danish Holstein cattle. The X-axis represents the distance of the chromosome expressed in Morgan according to the positions employed in this analysis. The Y-axis represents the likelihood ratio test-statistics of the QTL analysis.

FIG. 17: The haplotypes effect on mastitis resistance at 74.08 cM in Danish Red. The haplotypes effects are on the y-axis and haplotypes number is on x-axis. In the beginning there are the large clusters of dam haplotypes followed by the sire haplotypes.

FIG. 18: The haplotypes effect on mastitis resistance at 74.08 cM in Finnish Ayrshire. The haplotypes effects are on the y-axis and haplotypes number is on x-axis. In the beginning there are the large clusters of dam haplotypes followed by the sire haplotypes.

FIG. 19: The haplotypes effect on mastitis resistance at 74.08 cM in combined Danish Red and Finnish Ayrshire. The haplotypes effects are on the y-axis and haplotypes number is on x-axis. In the beginning there are the large clusters of dam haplotypes followed by the sire haplotypes.

FIG. 20: The haplotypes effect on mastitis resistance at 74.08 cM in combined Danish Red, Finnish Ayrshire and Swedish Red and White. The haplotypes effects are on the y-axis and haplotypes number is on x-axis. In the beginning there are the large clusters of dam haplotypes followed by the sire haplotypes.

FIG. 21: Genome scan of BTA11 in relation to the trait clinical mastitis characteristic of Finnish Ayrshire families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIG. 22: Genome scan of BTA11 in relation to the trait somatic cell score characteristic of Finnish Ayrshire families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIG. 23: Genome scan of BTA11 in relation to the trait clinical mastitis characteristic of Swedish Red and White families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIG. 24: Genome scan of BTA11 in relation to the trait somatic cell score characteristic of Swedish Red and White families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIG. 25: Genome scan of BTA11 in relation to the trait clinical mastitis characteristic of one Danish Red, eight Finnish Ayrshire and four Swedish Red and White families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIG. 26: Genome scan of BTA11 in relation to somatic cell score characteristic of one Danish Red, eight Finnish Ayrshire and four Swedish Red and White families. Numbers refer to ‘herdbook number’. The X-axis represents the distance of the chromosome expressed in Morgan according to the positions employed in this analysis. The Y-axis represents the test-statistics of the QTL analysis expressed in the F-value.

FIG. 27: QTL profile for the trait clinical mastitis showing LDLA/LD peak in Finnish Ayrshire cattle. The X-axis represents the distance of the chromosome expressed in Morgan according to the positions employed in this analysis. The Y-axis represents the likelihood ratio test-statistics of the QTL analysis.

FIG. 28: QTL profile for the trait clinical mastitis showing LDLA/LD peak with a 4-marker haplotype in Finnish Ayrshire cattle. The X-axis represents the distance of the chromosome expressed in Morgan according to the positions employed in this analysis. The Y-axis represents the likelihood ratio test-statistics of the QTL analysis.

FIG. 29: QTL profile for the trait somatic cell score showing LA, LDLA and LD profiles in Finnish Ayrshire cattle. The X-axis represents the distance of the chromosome expressed in Morgan according to the positions employed in this analysis. The Y-axis represents the likelihood ratio test-statistics of the QTL analysis.

FIG. 30: QTL profile for the trait somatic cell score showing LA, LDLA and LD profiles in Swedish Red and White cattle. The X-axis represents the distance of the chromosome expressed in Morgan according to the positions employed in this analysis. The Y-axis represents the likelihood ratio test-statistics of the QTL analysis.

FIG. 31: QTL profile for the trait clinical mastitis showing LA, LDLA and LD profiles in combined analysis of 14 families of Nordic Red breeds. The X-axis represents the distance of the chromosome expressed in Morgan according to the positions employed in this analysis. The Y-axis represents the likelihood ratio test-statistics of the QTL analysis.

FIG. 32: QTL profile for the trait somatic cell score showing QTL profiles in combined analysis of 14 families from three Nordic Red cattle. The X-axis represents the distance of the chromosome expressed in Morgan according to the positions employed in this analysis. The Y-axis represents the likelihood ratio test-statistics of the QTL analysis.

FIG. 33: The 4-marker haplotypes effect on clinical mastitis at 17.8 cM in Finnish Ayrshire. The haplotypes effects are on the y-axis and haplotypes number is on x-axis. In the beginning there are the large clusters of dam haplotypes followed by the sire haplotypes.

### DETAILED DESCRIPTION OF THE INVENTION

The present invention relates to genetic determinants of mastitis resistance in dairy cattle. The occurrence of mastitis, both clinical and sub-clinical mastitis involves substantial economic loss for the dairy industry. Therefore, it is of economic interest to identity those bovine subjects that have a genetic predisposition for mastitis resistance. Bovine subjects with such genetic predisposition are carriers of desired traits, which can be passed on to their offspring.

The term “bovine subject” refers to cattle of any breed and is meant to include both cows and bulls, whether adult or newborn animals. No particular age of the animals are denoted by this term. One example of a bovine subject is a member of the Holstein breed. In one preferred embodiment, the bovine subject is a member of the Holstein-Friesian cattle population. In another embodiment, the bovine subject is a member of the Holstein Swartbont cattle population. In another embodiment, the bovine subject is a member of the Deutsche Holstein Schwarzbunt cattle population. In another embodiment, the bovine subject is a member of the US Holstein cattle population. In one embodiment, the bovine subject is a member of the Red and White Holstein breed. In another embodiment, the bovine subject is a member of the Deutsche Holstein Schwarzbunt cattle population. In one embodiment, the bovine subject is a member of any family, which include members of the Holstein breed. In one preferred embodiment the bovine subject is a member of the Danish Red population. In another preferred embodiment the bovine subject is a member of the Finnish Ayrshire population. In yet another embodiment the bovine subject is a member of the Swedish Red and White population. In a further embodiment the bovine subject is a member of the Danish Holstein population. In another embodiment, the bovine subject is a member of the Swedish Red and White population. In yet another embodiment, the bovine subject is a member of the Nordic Red population.

In one embodiment of the present invention, the bovine subject is selected from the group consisting of Swedish Red and White, Danish Red, Finnish Ayrshire, Holstein-Friesian, Danish Holstein and Nordic Red. In another embodiment of the present invention, the bovine subject is selected from the group consisting of Finnish Ayrshire and Swedish Red and White cattle. In another embodiment of the present invention, the bovine subject is selected from the group consisting of Finnish Ayrshire and Swedish Red and White cattle.

In one embodiment, the bovine subject is selected from the group of breeds shown in table 1a

TABLE 1a
Breed names and breed codes assigned by ICAR
(International Committee for Animal Recording)

| | Breed | National Breed |
|---|---|---|
| Breed | Code | Names Annex |
| Abondance | AB | — |
| Tyrol Grey | AL | 2.2 |
| Angus | AN | 2.1 |
| Aubrac | AU | |
| Ayrshire | AY | 2.1 |
| Belgian Blue | BB | |
| Blonde d'Aquitaine | BD | |
| Beefmaster | BM | |
| Braford | BO | |
| Brahman | BR | |
| Brangus | BN | |
| Brown Swiss | BS | 2.1 |
| Chianina | CA | |
| Charolais | CH | |
| Dexter | DR | |
| Galloway | GA | 2.2 |
| Guernsey | GU | |
| Gelbvieh | GV | |
| Hereford, horned | HH | |
| Hereford, polled | HP | |
| Highland Cattle | HI | |
| Holstein | HO | 2.2 |
| Jersey | JE | |
| Limousin | LM | |
| Maine-Anjou | MA | |
| Murray-Grey | MG | |
| Montbéliard | MO | |
| Marchigiana | MR | |
| Normandy | NO** | |
| Piedmont | PI | 2.2 |
| Pinzgau | PZ | |
| European Red Dairy Breed | [RE]* | 2.1, 2.2 |
| Romagnola | RN | |
| Holstein, Red and White | RW*** | 2.2 |
| Salers | SL** | |
| Santa Gertrudis | SG | |
| South Devon | SD | |
| Shorthorn | [SH]* | 2.2 |
| Simmental | SM | 2.2 |
| Sahiwal | SW | |
| Tarentaise | TA | |
| Welsh Black | WB | |
| Buffalo (Bubalis bubalis) | BF | |

*new breed code
**change from earlier code because of existing code in France
***US proposal WW

In one embodiment, the bovine subject is a member of a breed selected from the group of breeds shown in table 1b

TABLE 1b
Breed names
National Breed Names

| English Name | National names | |
|---|---|---|
| Angus | Including | Aberdeen Angus |
| | | Canadian Angus |
| | | American Angus |
| | | German Angus |
| Ayrshire | Including | Ayrshine in |
| | | Australia |
| | | Canada |
| | | Colombia |
| | | Czech Republic |
| | | Finland |
| | | Kenya |
| | | New Zealand |
| | | Norway (NRF) |
| | | Russia |
| | | South Africa |
| | | Sweden (SRB) and SAB |
| | | UK |
| | | US |
| | | Zimbabwe |
| Belgian Blue | French: | Blane-bleu Belge |
| | Flemish: | Witblauw Ras van Belgie |
| Brown Swiss | German: | Braunvieh |
| | Italian: | Razza Bruna |
| | French: | Brune |
| | Spanish: | Bruna, Parda Alpina |
| | Serbo-Croatian: | Slovenacko belo |
| | Czech: | Hnedy Karpatsky |
| | Romanian: | Shivitskaja |
| | Russian: | Bruna |
| | Bulgarian: | BTjarska kafyava |
| European Red Dairy Breed | Including | Danish Red |
| | | Angeln |
| | | Swedish Red and White |
| | | Norwegian Red and White |
| | | Estonian Red |
| | | Latvian Brown |
| | | Lithuanian Red |
| | | Byelorus Red |
| | | Polish Red Lowland |

In one embodiment, the bovine subject is a member of a breed selected from the group of breeds shown in table 1c

TABLE 1c
Breed names
National Breed Names

| English Name | National names | |
|---|---|---|
| European Red Dairy Breed | | Ukrainian Polish Red |
| (continued) | | (French Rouge Flamande?) |
| | | (Belgian Flamande Rouge?) |
| Galloway: | Including | Black and Dun |
| | | Galloway |
| | | Belted Galloway |
| | | Red Galloway |
| | | White Galloway |
| Holstein, Black and White: | Dutch: | Holstein Swartbont |
| | German: | Deutsche Holstein, schwarzbunt |
| | Danish: | Sortbroget Dansk Malkekvaeg |
| | British: | Holstein Friesian |
| | Swedish: | Svensk Laglands Boskaap |
| | French: | Prim Holstein |
| | Italian: | Holstein Frisona |
| | Spanish: | Holstein Frisona |
| Holstein, Red and White | Dateh: | Holstein, roodbunt |
| | German: | Holstein, rotbunt |
| | Danish: | Roedbroget Dansk Malkekvaeg |
| Piedmont | Italian: | Piemontese |
| Shorthorn | Including | Dairy Shorthorn |
| | | Beef Shorthorn |
| | | Polled Shorthorn |
| Simmental | Including dual purpose and beef use | |
| | German: | Fleckvieh |
| | French: | Simmental Francaise |
| | Italian: | Razza Pezzata Rossa |
| | Czech: | Cesky strakaty |
| | Slovakian: | Slovensky strakaty |
| | Romanian: | Baltata romaneasca |
| | Russian: | Simmentalskaja |
| Tyrol Grey | German: | Tmoler Grauvieh |
| | | Oberimtaler Grauvieh |
| | | Ratisches Grauvieh |
| | Italian: | Razza Grigia Alpina |

The term “genetic marker” refers to a variable nucleotide sequence (polymorphism) of the DNA on the bovine chromosome and distinguishes one allele from another. The variable nucleotide sequence can be identified by methods known to a person skilled in the art for example by using specific oligonucleotides in for example amplification methods and/or observation of a size difference. However, the variable nucleotide sequence may also be detected by sequencing or for example restriction fragment length polymorphism analysis. The variable nucleotide sequence may be represented by a deletion, an insertion, repeats, and/or a point mutation.

One type of genetic marker is a microsatellite marker that is linked to a quantitative trait locus. Microsatellite markers refer to short sequences repeated after each other. In short sequences are for example one nucleotide, such as two nucleotides, for example three nucleotides, such as four nucleotides, for example five nucleotides, such as six nucleotides, for example seven nucleotides, such as eight nucleotides, for example nine nucleotides, such as ten nucleotides. However, changes sometimes occur and the number of repeats may increase or decrease. The specific definition and locus of the polymorphic microsatellite markers can be found in the USDA genetic map (Kappes et al. 1997; or by following the link to U.S. Meat Animal Research Center http://www.marc.usda.gov/).

It is furthermore appreciated that the nucleotide sequences of the genetic markers of the present invention are genetically linked to traits for mastitis resistance in a bovine subject. Consequently, it is also understood that a number of genetic markers may be generated from the nucleotide sequence of the DNA region(s) flanked by and including the genetic markers according to the method of the present invention.

The term ‘Quantitative trait locus (QTL)’ is a region of DNA that is associated with a particular trait (e.g., plant height). Though not necessarily genes themselves, QTLs are stretches of DNA that are closely linked to the genes that underlie the trait in question.

The term ‘mastitis’ is in the present application used to describe both the sub-clinical mastitis characterized for example by high somatic cell score (SCS) and clinical mastitis.

The terms ‘mastitis resistance’ and ‘resistance to mastitis’ are used interchangeable and relates to the fact that some bovine subjects are not as prone to mastitis as are other bovine subjects. When performing analyses of a number of bovine subjects as in the present invention in order to determine genetic markers that are associated with resistance to mastitis, the traits implying resistance to mastitis may be observed by the presence or absence of genetic markers linked to occurrence of clinical mastitis and/or sub-clinical mastitis in the bovine subjects analyzed. It is understood that mastitis resistance comprise resistance to traits, which affect udder health in the bovine subject or its off-spring. Thus, mastitis resistance of a bull is physically manifested by its female off-spring.

### Scoring for Mastitis Resistance

Daughters of bulls were scored for mastitis resistance and SCC. Somatic cell score (SCS) was defined as the mean of log¹⁰ transformed somatic cell count values (in 10,000/mL) obtained from the milk recording scheme. The mean was taken over the period 10 to 180 after calving. Estimated breeding values (EBV) for traits of sons were calculated using a single trait Best Linear Unbiased Prediction (BLUP) animal model ignoring family structure. These EBVs were used in the QTL analysis. The daughter registrations used in the individual traits were:

Clinical mastitis in Denmark: Treated cases of clinical mastitis in the period −5 to 50 days after 1^{st} calving.

Clinical mastitis in Sweden and Finland: Treated cases of clinical mastitis in the period −7 to 150 days after 1^{st} calving.

SCS in Denmark: Mean SCS in period 10-180 days after 1^{st} calving.

SCS in Sweden: Mean SCS in period 10-150 days after 1^{st} calving.

SCS in Finland: Mean SCS in period 10-305 days after 1^{st} calving.

In one embodiment of the present invention, the method and kit described herein relates to mastitis resistance. In another embodiment of the present invention, the method and kit described herein relates to resistance to clinical mastitis. In another embodiment, the method and kit of the present invention pertains to resistance to sub-clinical mastitis, such as detected by somatic cell counts. In yet another embodiment, the method and kit of the present invention primarily relates to resistance to clinical mastitis in combination with resistance to sub-clinical mastitis such as detected by somatic cell counts.

### Sample

The method according to the present invention includes analyzing a sample of a bovine subject, wherein said sample may be any suitable sample capable of providing the bovine genetic material for use in the method. The bovine genetic material may for example be extracted, isolated and purified if necessary from a blood sample, a tissue samples (for example spleen, buccal smears), clipping of a body surface (hairs or nails), milk and/or semen. The samples may be fresh or frozen.

The sequence polymorphisms of the invention comprise at least one nucleotide difference, such as at least two nucleotide differences, for example at least three nucleotide differences, such as at least four nucleotide differences, for example at least five nucleotide differences, such as at least six nucleotide differences, for example at least seven nucleotide differences, such as at least eight nucleotide differences, for example at least nine nucleotide differences, such as 10 nucleotide differences. The nucleotide differences comprise nucleotide differences, deletion and/or insertion or any combination thereof.

### Grand Daughter Design

The grand daughter design includes analysing data from DNA-based markers for grand sires that have been used extensively in breeding and for sons of grand sires where the sons have produced offspring. The phenotypic data that are to be used together with the DNA-marker data are derived from the daughters of the sons. Such phenotypic data could be for example milk production features, features relating to calving, meat quality, or disease. One group of daughters have inherited one allele from their father whereas a second group of daughters have inherited the other allele form their father. By comparing data from the two groups information can be gained whether a fragment of a particular chromosome is harbouring one or more genes that affect the trait in question. It may be concluded whether a QTL is present within this fragment of the chromosome.

A prerequisite for performing a grand daughter design is the availability of detailed phenotypic data. In the present invention such data have been available (http://www.lr.dk/kvaeg/diverse/principles.pdf).

QTL is a short form of quantitative trait locus. Genes conferring quantitative traits to an individual may be found in an indirect manner by observing pieces of chromosomes that act as if one or more gene(s) is located within that piece of the chromosome.

In contrast, DNA markers can be used directly to provide information of the traits passed on from parents to one or more of their off spring when a number of DNA markers on a chromosome have been determined for one or both parents and their off-spring. The markers may be used to calculate the genetic history of the chromosome linked to the DNA markers.

### Chromosomal Regions and Markers

BTA is short for Bos taurus autosome.

One aspect of the present invention relates to a method for determining the resistance to mastitis in a bovine subject, comprising detecting in a sample from said bovine subject the presence or absence of at least one genetic marker that is linked to at least one trait indicative of mastitis resistance, wherein said at least one genetic marker is located on the bovine chromosome BTA9 in the region flanked by and including the polymorphic microsatellite markers C6orf93 and inra084 and/or BTA11 in the region flanked by and including the polymorphic microsatellite markers HELMTT43 and BM3501, wherein the presence or absence of said at least one genetic marker is indicative of mastitis resistance of said bovine subject or off-spring therefrom.

Due to the concept of linkage disequilibrium as described herein the present invention also relates to determining the resistance to mastitis in a bovine subject, wherein the at least one genetic marker is linked to a bovine trait for resistance to mastitis.

In order to determine resistance to mastitis in a bovine subject, it is appreciated that more than one genetic marker may be employed in the present invention. For example the at least one genetic marker may be a combination of at least two or more genetic markers such that the accuracy may be increased, such as at least three genetic markers, for example four genetic markers, such as at least five genetic markers, for example six genetic markers, such as at least seven genetic markers, for example eight genetic markers, such as at least nine genetic markers, for example ten genetic markers.

The at least one genetic marker may be located on at least one bovine chromosome, such as two chromosomes, for example three chromosomes, such as four chromosomes, for example five chromosomes, and/or such as six chromosomes. The at least one genetic marker may be located on the bovine chromosome 9.

However, the at least one genetic marker may a combination of markers located on different chromosomes.

The at least one genetic marker is selected from any of the individual markers of the tables shown herein.

In one embodiment of the invention the at least one genetic marker is located on the bovine chromosome BTA9. In one embodiment the at least one genetic marker is located on the bovine chromosome BTA9 in the region flanked by and including the markers c6orf93 and rgs17. In one embodiment of the present invention, the at least one genetic marker is located in the region from about 69.35 cM to about 79.8 cM (according to the positions employed in this analysis) on the bovine chromosome BTA9. The at least one genetic marker is selected from the group of markers shown in Table 1d.

TABLE 1d

| | Position employed in | Relative position (cM) |
|---|---|---|
| Marker on BTA9 | analysis (cM) | http://www.marc.usda.gov/ |
| C6orf93* | 69.35 | — |
| DIK 4986 | 69.4 | 84.258 |
| mm12e6* | 69.45 | 84.258 |
| PEX3* | 69.5 | — |
| DEAD21 | 69.55 | — |
| BMS2251 | 71.3 | 86.58 |
| EPM2A* | 72.1 | |
| BM7234 | 72.3 | 88.136 |
| BM4208 | 73.9 | 90.69 |
| BMS2819 | 73.95 | 90.98 |
| INRA144 | 74.2 | 90.98 |
| INRA084 | 74.5 | 90.98 |
| rgs17* | 79.8 | — |

*denotes markers that are not listed on the MARC marker map at BTA9.

In another embodiment of the invention the at least one genetic marker is located on the bovine chromosome BTA9. In one embodiment the at least one genetic marker is located on the bovine chromosome BTA9 in the region flanked by and including the markers c6orf93 and inra084. In one embodiment of the present invention, the at least one genetic marker is located in the region from about 69.35 cM to about 74.5 cM (according to the positions employed in this analysis) on the bovine chromosome BTA9. According to the MARC marker map the position of the genetic marker inra084 is 90.98. The at least one genetic marker is selected from the group of markers shown in Table 2.

TABLE 2

| | Position employed in | Relative position (cM) |
|---|---|---|
| Marker on BTA9 | analysis (cM) | http://www.marc.usda.gov/ |
| C6orf93* | 69.35 | — |
| DIK 4986 | 69.4 | 84.258 |
| mm12e6* | 69.45 | 84.258 |
| PEX3* | 69.5 | — |
| DEAD21 | 69.55 | — |
| BMS2251 | 71.3 | 86.58 |
| EPM2A* | 72.1 | |
| BM7234 | 72.3 | 88.136 |
| BM4208 | 73.9 | 90.69 |
| BMS2819 | 73.95 | 90.98 |
| INRA144 | 74.2 | 90.98 |
| INRA084 | 74.5 | 90.98 |

*denotes markers that are not listed on the MARC marker map at BTA9.

In a further embodiment the at least one genetic marker is located on the bovine chromosome BTA9 in the region flanked by and including the markers bms2251 and inra084. In one embodiment of the present invention, the at least one genetic marker is located in the region from about 71.3 cM to about 74.5 cM on the bovine chromosome BTA9 (according to the positions employed in this analysis) and from about 86.58 cM to about 90.98 cM according to the to the MARC marker map. The at least one genetic marker is selected from the group of markers shown in Table 3.

TABLE 3

| | Position employed in | Relative position (cM) |
|---|---|---|
| Marker on BTA9 | analysis (cM) | http://www.marc.usda.gov/ |
| BMS2251 | 71.3 | 86.58 |
| EPM2A* | 72.1 | |
| BM7234 | 72.3 | 88.136 |
| BM4208 | 73.9 | 90.69 |
| BMS2819 | 73.95 | 90.98 |
| INRA144 | 74.2 | 90.98 |
| INRA084 | 74.5 | 90.98 |

*denotes markers that are not listed on the MARC marker map at BTA9.

In another embodiment the at least one genetic marker is located on the bovine chromosome BTA9 in the region flanked by and including the markers bms2251 and inra144. In one embodiment of the present invention, the at least one genetic marker is located in the region from about 71.3 cM to about 74.2 cM on the bovine chromosome BTA9 (according to the positions employed in this analysis) and from about 86.58 cM to about 90.98 cM according to the to the MARC marker map. The at least one genetic marker is selected from the group of markers shown in Table 4.

TABLE 4

| | Position employed in | Relative position (cM) |
|---|---|---|
| Marker on BTA9 | analysis (cM) | http://www.marc.usda.gov/ |
| BMS2251 | 71.3 | 86.58 |
| EPM2A* | 72.1 | |
| BM7234 | 72.3 | 88.136 |
| BM4208 | 73.9 | 90.69 |
| BMS2819 | 73.95 | 90.98 |
| INRA144 | 74.2 | 90.98 |

*denotes markers that are not listed on the MARC marker map at BTA9.

In yet another embodiment the at least one genetic marker is located on the bovine chromosome BTA9 in the region flanked by and including the markers bm7234 and inra084. In one embodiment of the present invention, the at least one genetic marker is located in the region from about 72.3 cM to about 74.5 cM on the bovine chromosome BTA9 (according to the positions employed in this analysis) and from about 88.136 cM to about 90.98 cM according to the to the MARC marker map. The at least one genetic marker is selected from the group of markers shown in Table 5.

TABLE 5

| | Position employed in | Relative position (cM) |
|---|---|---|
| Marker on BTA9 | analysis (cM) | http://www.marc.usda.gov/ |
| BM7234 | 72.3 | 88.136 |
| BM4208 | 73.9 | 90.69 |
| BMS2819 | 73.95 | 90.98 |
| INRA144 | 74.2 | 90.98 |
| INRA084 | 74.5 | 90.98 |

*denotes markers that are not listed on the MARC marker map at BTA9.

In a further embodiment the at least one genetic marker is located on the bovine chromosome BTA9 in the region flanked by and including the markers bm7234 and inra144. In one embodiment of the present invention, the at least one genetic marker is located in the region from about 72.3 cM to about 74.2 cM on the bovine chromosome BTA9 (according to the positions employed in this analysis) and from about 88.136 cM to about 90.98 cM according to the to the MARC marker map. The at least one genetic marker is selected from the group of markers shown in Table 6.

TABLE 6

| | Position employed in | Relative position (cM) |
|---|---|---|
| Marker on BTA9 | analysis (cM) | http://www.marc.usda.gov/ |
| BM7234 | 72.3 | 88.136 |
| BM4208 | 73.9 | 90.69 |
| BMS2819 | 73.95 | 90.98 |
| INRA144 | 74.2 | 90.98 |

*denotes markers that are not listed on the MARC marker map at BTA9.

In yet a further embodiment the at least one genetic marker is located on the bovine chromosome BTA9 in the region flanked by and including the markers bm7234 and bms2819. In one embodiment of the present invention, the at least one genetic marker is located in the region from about 72.3 cM to about 73.95 cM on the bovine chromosome BTA9 (according to the positions employed in this analysis) and from about 88.136 cM to about 90.98 cM according to the to the MARC marker map. The at least one genetic marker is selected from the group of markers shown in Table 7.

TABLE 7

| | Position employed in | Relative position (cM) |
|---|---|---|
| Marker on BTA9 | analysis (cM) | http://www.marc.usda.gov/ |
| BM7234 | 72.3 | 88.136 |
| BM4208 | 73.9 | 90.69 |
| BMS2819 | 73.95 | 90.98 |

*denotes markers that are not listed on the MARC marker map at BTA9.

In another embodiment the at least one genetic marker is located on the bovine chromosome BTA9 in the region flanked by and including the markers bm7234 and bm4208. In one embodiment of the present invention, the at least one genetic marker is located in the region from about 72.3 cM to about 73.9 cM on the bovine chromosome BTA9 (according to the positions employed in this analysis) and from about 88.136 cM to about 90.69 cM according to the to the MARC marker map. The at least one genetic marker is selected from the group of markers shown in Table 8.

TABLE 8

| | Position employed in | Relative position (cM) |
|---|---|---|
| Marker on BTA9 | analysis (cM) | http://www.marc.usda.gov/ |
| BM7234 | 72.3 | 88.136 |
| BM4208 | 73.9 | 90.69 |

*denotes markers that are not listed on the MARC marker map at BTA9.

In a further embodiment the at least one genetic marker is located on the bovine chromosome BTA9 in the region flanked by and including the markers bms2819 and inra144. In one embodiment of the present invention, the at least one genetic marker is located in the region from about 73.95 cM to about 74.2 cM on the bovine chromosome BTA9 (according to the positions employed in this analysis) and from about 90.98 cM to about 90.98 cM according to the to the MARC marker map. The at least one genetic marker is selected from the group of markers shown in Table 9.

TABLE 9

| | Position employed in | Relative position (cM) |
|---|---|---|
| Marker on BTA9 | analysis (cM) | http://www.marc.usda.gov/ |
| BMS2819 | 73.95 | 90.98 |
| INRA144 | 74.2 | 90.98 |

*denotes markers that are not listed on the MARC marker map at BTA9.

In another embodiment of the present invention the at least one genetic marker is located on the bovine chromosome BTA9 in the region flanked by and including the markers bms2819 and inra084. In one embodiment of the present invention, the at least one genetic marker is located in the region from about 73.95 cM to about 74.5 cM on the bovine chromosome BTA9 (according to the positions employed in this analysis) and from about 90.98 cM to about 90.98 cM according to the to the MARC marker map. The at least one genetic marker is selected from the group of markers shown in Table 10.

TABLE 10

| | Position employed in | Relative position (cM) |
|---|---|---|
| Marker on BTA9 | analysis (cM) | http://www.marc.usda.gov/ |
| BMS2819 | 73.95 | 90.98 |
| INRA144 | 74.2 | 90.98 |
| INRA084 | 74.5 | 90.98 |

*denotes markers that are not listed on the MARC marker map at BTA9.

In another embodiment of the present invention the at least one genetic marker is located on the bovine chromosome BTA9 in the region flanked by and including the markers bm4208 and inra144. In one embodiment of the present invention, the at least one genetic marker is located in the region from about 73.9 cM to about 74.2 cM on the bovine chromosome BTA9 (according to the positions employed in this analysis) and from about 90.69 cM to about 90.98 cM according to the to the MARC marker map. The at least one genetic marker is selected from the group of markers shown in Table 11.

TABLE 11

| | Position employed in | Relative position (cM) |
|---|---|---|
| Marker on BTA9 | analysis (cM) | http://www.marc.usda.gov/ |
| BM4208 | 73.9 | 90.69 |
| BMS2819 | 73.95 | 90.98 |
| INRA144 | 74.2 | 90.98 |
| INRA084 | 74.5 | 90.98 |

*denotes markers that are not listed on the MARC marker map at BTA9.

In yet another embodiment of the present invention the at least one genetic marker is located on the bovine chromosome BTA9 in the region flanked by and including the markers inra144 and inra084. In one embodiment of the present invention, the at least one genetic marker is located in the region from about 74.2 cM to about 74.5 cM on the bovine chromosome BTA9 (according to the positions employed in this analysis) and from about 90.98 cM to about 90.98 cM according to the to the MARC marker map. The at least one genetic marker is selected from the group of markers shown in Table 12.

TABLE 12

| | Position employed in | Relative position (cM) |
|---|---|---|
| Marker on BTA9 | analysis (cM) | http://www.marc.usda.gov/ |
| INRA144 | 74.2 | 90.98 |
| INRA084 | 74.5 | 90.98 |

*denotes markers that are not listed on the MARC marker map at BTA9.

In a further embodiment the at least one genetic marker is located on the bovine chromosome BTA9 in the region flanked by and including the markers bms2251 and bm7234. In one embodiment of the present invention, the at least one genetic marker is located in the region from about 71.3 cM to about 72.3 cM on the bovine chromosome BTA9 (according to the positions employed in this analysis) and from about 86.58 cM to about 88.136 cM according to the to the MARC marker map. The at least one genetic marker is selected from the group of markers shown in Table 13.

TABLE 13

| | Position employed in | Relative position (cM) |
|---|---|---|
| Marker on BTA9 | analysis (cM) | http://www.marc.usda.gov/ |
| BMS2251 | 71.3 | 86.58 |
| EPM2A* | 72.1 | |
| BM7234 | 72.3 | 88.136 |

*denotes markers that are not listed on the MARC marker map at BTA9.

In yet a further embodiment the at least one genetic marker is located on the bovine chromosome BTA9 in the region flanked by and including the markers EPM2A and bm7234. In one embodiment of the present invention, the at least one genetic marker is located in the region from about 72.1 cM to about 72.3 cM on the bovine chromosome BTA9 (according to the positions employed in this analysis) and for bm7234 about 88.136 cM according to the to the MARC marker map. The at least one genetic marker is selected from the group of markers shown in Table 14.

TABLE 14

| | Position employed in | Relative position (cM) |
|---|---|---|
| Marker on BTA9 | analysis (cM) | http://www.marc.usda.gov/ |
| EPM2A* | 72.1 | |
| BM7234 | 72.3 | 88.136 |

*denotes markers that are not listed on the MARC marker map at BTA9.

In one embodiment of the invention the at least one genetic marker is located on the bovine chromosome BTA9. In one embodiment the at least one genetic marker is located on the bovine chromosome BTA9 in the region flanked by and including the markers inra144 and rgs17. In one embodiment of the present invention, the at least one genetic marker is located in the region from about 74.2 cM to about 79.8 cM (according to the positions employed in this analysis) on the bovine chromosome BTA9 where the position of inra144 according to the MARC marker map is 90.98 cM. The at least one genetic marker is selected from the group of markers shown in Table 15.

TABLE 15

| | Position employed in | Relative position (cM) |
|---|---|---|
| Marker on BTA9 | analysis (cM) | http://www.marc.usda.gov/ |
| INRA144 | 74.2 | 90.98 |
| INRA084 | 74.5 | 90.98 |
| rgs17* | 79.8 | — |

*denotes markers that are not listed on the MARC marker map at BTA9.

In another embodiment of the invention the at least one genetic marker is located on the bovine chromosome BTA9. In one embodiment the at least one genetic marker is located on the bovine chromosome BTA9 in the region flanked by and including the markers inra084 and rgs17. In one embodiment of the present invention, the at least one genetic marker is located in the region from about 74.5 cM to about 79.8 cM (according to the positions employed in this analysis) on the bovine chromosome BTA9 and where the position of inra084 according to the MARC marker map is 90.68 cM. The at least one genetic marker is selected from the group of markers shown in Table 16.

TABLE 16

| | Position employed in | Relative position (cM) |
|---|---|---|
| Marker on BTA9 | analysis (cM) | http://www.marc.usda.gov/ |
| INRA084 | 74.5 | 90.98 |
| rgs17* | 79.8 | — |

*denotes markers that are not listed on the MARC marker map at BTA9.

In one embodiment of the invention the at least one genetic marker is located on the bovine chromosome BTA11. In one embodiment the at least one genetic marker is located on the bovine chromosome BTA11 in the region flanked by and including the markers HELMTT43 and BM3501. In one embodiment of the present invention, the at least one genetic marker is located in the region from about 2.249 cM to about 97.223 cM on the bovine chromosome BTA11. The at least one genetic marker is selected from the group of markers shown in Table 17.

TABLE 17

| | | Relative |
|---|---|---|
| | Position employed | position (cM) |
| Marker on | in analysis | http://www.marc.usda.gov/ |
| BTA11 | (cM) | genome/cattle/cattle.html |
| HELMTT43 | 0.0 | 2.249 |
| ZAP70* | 5.4 | — |
| MAP4K4* | 10.5 | — |
| IL18RA* | 12.3 | — |
| MNB-40 | 16.0 | 19.440 |
| AUP1* | 17.6 | — |
| BM716 | 17.9 | 19.440 |
| DIK2653 | 18.1 | 20.135 |
| BMS2569 | 18.3 | 21.082 |
| BMS2325 | 18.5 | 21.082 |
| BMS1953 | 18.8 | 21.537 |
| DIK4637 | 19.4 | 22.527 |
| UMBTL103 | 21.8 | 23.829 |
| BP38 | 22.6 | 24.617 |
| MNB-70 | 22.8 | 24.617 |
| BM2818 | 26.5 | 30.009 |
| BM304 | 30.0 | 33.597 |
| INRA177 | 32.5 | 35.098 |
| UMBTL20 | 32.7 | 34.802 |
| RM96* | 38.0 | — |
| INRA131 | 43.6 | 47.289 |
| BM7169 | 46.8 | 50.312 |
| BMS1716 | 50.2 | 54.581 |
| BM6445 | 55.1 | 61.570 |
| CD8B* | 56.9 | — |
| MB110 | 59.6 | 68.679 |
| MS2177 | 61.0 | 69.415 |
| HELMTT44* | 61.2 | — |
| DIK5170 | 61.4 | 70.143 |
| RM150 | 61.8 | 70.143 |
| TGLA58 | 63.1 | 73.136 |
| TGLA340 | 65.8 | 75.208 |
| BM8118 | 67.2 | 77.063 |
| BMS2047 | 68.8 | 78.457 |
| BMS1048 | 69.5 | 81.065 |
| BMS989 | 78.9 | 92.179 |
| BM3501 | 85.2 | 97.223 |

*These markers are not listed in the MARC marker map of BTA11, but identified by the present inventors. This is applicable throughout the tables herein.

In another embodiment of the invention the at least one genetic marker is located on the bovine chromosome BTA11. In one embodiment the at least one genetic marker is located on the bovine chromosome BTA11 in the region flanked by and including the markers HELMTT43 and INRA177. In one embodiment of the present invention, the at least one genetic marker is located in the region from about 2.249 cM to about 35.098 cM on the bovine chromosome BTA11. The at least one genetic marker is selected from the group of markers shown in Table 18.

TABLE 18

| | Position employed | Relative position (cM) |
|---|---|---|
| Marker on | in analysis | http://www.marc.usda.gov/genome/ |
| BTA11 | (cM) | cattle/cattle.html |
| HELMTT43 | 0.0 | 2.249 |
| ZAP70* | 5.4 | — |
| MAP4K4* | 10.5 | — |
| IL18RA* | 12.3 | — |
| MNB-40 | 16.0 | 19.440 |
| AUP1* | 17.6 | — |
| BM716 | 17.9 | 19.440 |
| DIK2653 | 18.1 | 20.135 |
| BMS2569 | 18.3 | 21.082 |
| BMS2325 | 18.5 | 21.082 |
| BMS1953 | 18.8 | 21.537 |
| DIK4637 | 19.4 | 22.527 |
| UMBTL103 | 21.8 | 23.829 |
| BP38 | 22.6 | 24.617 |
| MNB-70 | 22.8 | 24.617 |
| BM2818 | 26.5 | 30.009 |
| BM304 | 30.0 | 33.597 |
| INRA177 | 32.5 | 35.098 |

In a further embodiment the at least one genetic marker is located on the bovine chromosome BTA11 in the region flanked by and including the markers HELMTT43 and MNB-70. In one embodiment of the present invention, the at least one genetic marker is located in the region from about 2.249 cM to about 24.617 cM on the bovine chromosome BTA11. The at least one genetic marker is selected from the group of markers shown in Table 19.

TABLE 19

| | Position employed | Relative position (cM) |
|---|---|---|
| Marker on | in analysis | http://www.marc.usda.gov/genome/ |
| BTA11 | (cM) | cattle/cattle.html |
| HELMTT43 | 0.0 | 2.249 |
| ZAP70* | 5.4 | — |
| MAP4K4* | 10.5 | — |
| IL18RA* | 12.3 | — |
| MNB-40 | 16.0 | 19.440 |
| AUP1* | 17.6 | — |
| BM716 | 17.9 | 19.440 |
| DIK2653 | 18.1 | 20.135 |
| BMS2569 | 18.3 | 21.082 |
| BMS2325 | 18.5 | 21.082 |
| BMS1953 | 18.8 | 21.537 |
| DIK4637 | 19.4 | 22.527 |
| UMBTL103 | 21.8 | 23.829 |
| BP38 | 22.6 | 24.617 |
| MNB-70 | 22.8 | 24.617 |

In another embodiment the at least one genetic marker is located on the bovine chromosome BTA11 in the region flanked by and including the markers MNB-40 and MNB-70. In one embodiment of the present invention, the at least one genetic marker is located in the region from about 19.440 cM to about 24.617 cM on the bovine chromosome BTA11. The at least one genetic marker is selected from the group of markers shown in Table 20.

TABLE 20

| | Position employed | Relative position (cM) |
|---|---|---|
| Marker on | in analysis | http://www.marc.usda.gov/genome/ |
| BTA11 | (cM) | cattle/cattle.html |
| MNB-40 | 16.0 | 19.440 |
| AUP1* | 17.6 | — |
| BM716 | 17.9 | 19.440 |
| DIK2653 | 18.1 | 20.135 |
| BMS2569 | 18.3 | 21.082 |
| BMS2325 | 18.5 | 21.082 |
| BMS1953 | 18.8 | 21.537 |
| DIK4637 | 19.4 | 22.527 |
| UMBTL103 | 21.8 | 23.829 |
| BP38 | 22.6 | 24.617 |
| MNB-70 | 22.8 | 24.617 |

In yet another embodiment the at least one genetic marker is located on the bovine chromosome BTA11 in the region flanked by and including the markers BP38 and INRA131. In one embodiment of the present invention, the at least one genetic marker is located in the region from about 24.617 cM to about 47.289 cM on the bovine chromosome BTA11. The at least one genetic marker is selected from the group of markers shown in Table 21.

TABLE 21

| | Position employed | Relative position (cM) |
|---|---|---|
| Marker on | in analysis | http://www.marc.usda.gov/genome/ |
| BTA11 | (cM) | cattle/cattle.html |
| BP38 | 22.6 | 24.617 |
| MNB-70 | 22.8 | 24.617 |
| BM2818 | 26.5 | 30.009 |
| BM304 | 30.0 | 33.597 |
| INRA177 | 32.5 | 35.098 |
| UMBTL20 | 32.7 | 34.802 |
| RM96* | 38.0 | — |
| INRA131 | 43.6 | 47.289 |

In a further embodiment the at least one genetic marker is located on the bovine chromosome BTA11 in the region flanked by and including the markers BM2818 and INRA177. In one embodiment of the present invention, the at least one genetic marker is located in the region from about 30.009 cM to about 35.098 cM on the bovine chromosome BTA11. The at least one genetic marker is selected from the group of markers shown in Table 22.

TABLE 22

| | Position employed | Relative position (cM) |
|---|---|---|
| Marker on | in analysis | http://www.marc.usda.gov/genome/ |
| BTA11 | (cM) | cattle/cattle.html |
| BM2818 | 26.5 | 30.009 |
| BM304 | 30.0 | 33.597 |
| INRA177 | 32.5 | 35.098 |

In yet a further embodiment the at least one genetic marker is located on the bovine chromosome BTA11 in the region flanked by and including the markers BMS1953 and BM2818. In one embodiment of the present invention, the at least one genetic marker is located in the region from about 21.537 cM to about 30.009 cM on the bovine chromosome BTA11. The at least one genetic marker is selected from the group of markers shown in Table 23.

TABLE 23

| | Position employed | Relative position (cM) |
|---|---|---|
| Marker on | in analysis | http://www.marc.usda.gov/genome/ |
| BTA11 | (cM) | cattle/cattle.html |
| BMS1953 | 18.8 | 21.537 |
| DIK4637 | 19.4 | 22.527 |
| UMBTL103 | 21.8 | 23.829 |
| BP38 | 22.6 | 24.617 |
| MNB-70 | 22.8 | 24.617 |
| BM2818 | 26.5 | 30.009 |

In another embodiment the at least one genetic marker is located on the bovine chromosome BTA11 in the region flanked by and including the markers HELMTT43 and ZAP70. In one embodiment of the present invention, the at least one genetic marker is located in the region from about 2.249 cM (according to the MARC marker map) to about 5.4 cM (according to the positions employed in this analysis) on the bovine chromosome BTA11. The at least one genetic marker is selected from the group of markers shown in Table 24.

TABLE 24

| | Position employed | Relative position (cM) |
|---|---|---|
| Marker on | in analysis | http://www.marc.usda.gov/genome/ |
| BTA11 | (cM) | cattle/cattle.html |
| HELMTT43 | 0.0 | 2.249 |
| ZAP70* | 5.4 | — |

In a further embodiment the at least one genetic marker is located on the bovine chromosome BTA11 in the region flanked by and including the markers ZAP70 and IL18RA. In one embodiment of the present invention, the at least one genetic marker is located in the region from about 5.4 cM to about 12.3 cM on the bovine chromosome BTA11 (according to the positions employed in this analysis). The at least one genetic marker is selected from the group of markers shown in Table 25.

TABLE 25

| | Position employed | Relative position (cM) |
|---|---|---|
| Marker on | in analysis | http://www.marc.usda.gov/genome/ |
| BTA11 | (cM) | cattle/cattle.html |
| ZAP70* | 5.4 | — |
| MAP4K4* | 10.5 | — |
| IL18RA* | 12.3 | — |

In another embodiment of the present invention the at least one genetic marker is located on the bovine chromosome BTA11 in the region flanked by and including the markers INRA131 and BM6445. In one embodiment of the present invention, the at least one genetic marker is located in the region from about 47.289 cM to about 61.570 cM on the bovine chromosome BTA11. The at least one genetic marker is selected from the group of markers shown in Table 26.

TABLE 26

| | Position employed | Relative position (cM) |
|---|---|---|
| Marker on | in analysis | http://www.marc.usda.gov/genome/ |
| BTA11 | (cM) | cattle/cattle.html |
| INRA131 | 43.6 | 47.289 |
| BM7169 | 46.8 | 50.312 |
| BMS1716 | 50.2 | 54.581 |
| BM6445 | 55.1 | 61.570 |

In another embodiment of the present invention the at least one genetic marker is located on the bovine chromosome BTA11 in the region flanked by and including the markers BM304 and BM7169. In one embodiment of the present invention, the at least one genetic marker is located in the region from about 33.597 cM to about 50.312 cM on the bovine chromosome BTA11. The at least one genetic marker is selected from the group of markers shown in Table 27.

TABLE 27

| | Position employed | Relative position (cM) |
|---|---|---|
| Marker on | in analysis | http://www.marc.usda.gov/genome/ |
| BTA11 | (cM) | cattle/cattle.html |
| BM304 | 30.0 | 33.597 |
| INRA177 | 32.5 | 35.098 |
| UMBTL20 | 32.7 | 34.802 |
| RM96* | 38.0 | — |
| INRA131 | 43.6 | 47.289 |
| BM7169 | 46.8 | 50.312 |

In yet another embodiment of the present invention the at least one genetic marker is located on the bovine chromosome BTA11 in the region flanked by and including the markers BM7169 and DIK5170. In one embodiment of the present invention, the at least one genetic marker is located in the region from about 50.312 cM to about 70.143 cM on the bovine chromosome BTA11. The at least one genetic marker is selected from the group of markers shown in Table 28.

TABLE 28

| | Position employed | Relative position (cM) |
|---|---|---|
| Marker on | in analysis | http://www.marc.usda.gov/ |
| BTA11 | (cM) | genome/cattle/cattle.html |
| BM7169 | 46.8 | 50.312 |
| BMS1716 | 50.2 | 54.581 |
| BM6445 | 55.1 | 61.570 |
| CD8B* | 56.9 | — |
| MB110 | 59.6 | 68.679 |
| MS2177 | 61.0 | 69.415 |
| HELMTT44* | 61.2 | — |
| DIK5170 | 61.4 | 70.143 |

In a further embodiment the at least one genetic marker is located on the bovine chromosome BTA11 in the region flanked by and including the markers BM6445 and BMS1048. In one embodiment of the present invention, the at least one genetic marker is located in the region from about 61.570 cM to about 81.065 cM on the bovine chromosome BTA11. The at least one genetic marker is selected from the group of markers shown in Table 29.

TABLE 29

| | Position employed | Relative position (cM) |
|---|---|---|
| Marker on | in analysis | http://www.marc.usda.gov/ |
| BTA11 | (cM) | genome/cattle/cattle.html |
| BM6445 | 55.1 | 61.570 |
| CD8B* | 56.9 | — |
| MB110 | 59.6 | 68.679 |
| MS2177 | 61.0 | 69.415 |
| HELMTT44* | 61.2 | — |
| DIK5170 | 61.4 | 70.143 |
| RM150 | 61.8 | 70.143 |
| TGLA58 | 63.1 | 73.136 |
| TGLA340 | 65.8 | 75.208 |
| BM8118 | 67.2 | 77.063 |
| BMS2047 | 68.8 | 78.457 |
| BMS1048 | 69.5 | 81.065 |

In yet a further embodiment the at least one genetic marker is located on the bovine chromosome BTA11 in the region flanked by and including the markers MB110 and BMS2047. In one embodiment of the present invention, the at least one genetic marker is located in the region from about 68.679 cM to about 78.457 cM on the bovine chromosome BTA11. The at least one genetic marker is selected from the group of markers shown in Table 30.

TABLE 30

| | Position employed | Relative position (cM) |
|---|---|---|
| Marker on | in analysis | http://www.marc.usda.gov/ |
| BTA11 | (cM) | genome/cattle/cattle.html |
| MB110 | 59.6 | 68.679 |
| MS2177 | 61.0 | 69.415 |
| HELMTT44* | 61.2 | — |
| DIK5170 | 61.4 | 70.143 |
| RM150 | 61.8 | 70.143 |
| TGLA58 | 63.1 | 73.136 |
| TGLA340 | 65.8 | 75.208 |
| BM8118 | 67.2 | 77.063 |
| BMS2047 | 68.8 | 78.457 |

In one embodiment of the invention the at least one genetic marker is located on the bovine chromosome BTA11. In one embodiment the at least one genetic marker is located on the bovine chromosome BTA11 in the region flanked by and including the markers IL18RA and BM2818. In one embodiment of the present invention, the at least one genetic marker is located in the region from about 12.3 cM (according to the positions employed in this analysis) to about 30.009 cM (according to the MARC marker map) on the bovine chromosome BTA11. The at least one genetic marker is selected from the group of markers shown in Table 31.

TABLE 31

| | Position employed | Relative position (cM) |
|---|---|---|
| Marker on | in analysis | http://www.marc.usda.gov/ |
| BTA11 | (cM) | genome/cattle/cattle.html |
| IL18RA* | 12.3 | — |
| MNB-40 | 16.0 | 19.440 |
| AUP1* | 17.6 | — |
| BM716 | 17.9 | 19.440 |
| DIK2653 | 18.1 | 20.135 |
| BMS2569 | 18.3 | 21.082 |
| BMS2325 | 18.5 | 21.082 |
| BMS1953 | 18.8 | 21.537 |
| DIK4637 | 19.4 | 22.527 |
| UMBTL103 | 21.8 | 23.829 |
| BP38 | 22.6 | 24.617 |
| MNB-70 | 22.8 | 24.617 |
| BM2818 | 26.5 | 30.009 |

In another embodiment of the invention the at least one genetic marker is located on the bovine chromosome BTA11. In one embodiment the at least one genetic marker is located on the bovine chromosome BTA11 in the region flanked by and including the markers BM2818 and BM7169. In one embodiment of the present invention, the at least one genetic marker is located in the region from about 30.009 cM to about 50.312 cM on the bovine chromosome BTA11. The at least one genetic marker is selected from the group of markers shown in Table 32.

TABLE 32

| | Position employed | Relative position (cM) |
|---|---|---|
| Marker on | in analysis | http://www.marc.usda.gov/ |
| BTA11 | (cM) | genome/cattle/cattle.html |
| BM2818 | 26.5 | 30.009 |
| BM304 | 30.0 | 33.597 |
| INRA177 | 32.5 | 35.098 |
| UMBTL20 | 32.7 | 34.802 |
| RM96* | 38.0 | — |
| INRA131 | 43.6 | 47.289 |
| BM7169 | 46.8 | 50.312 |

In another embodiment of the invention the at least one genetic marker is located on the bovine chromosome BTA11. In one embodiment the at least one genetic marker is located on the bovine chromosome BTA11 in the region flanked by and including the markers MAP4K4 and BM2818. In one embodiment of the present invention, the at least one genetic marker is located in the region from about 10.5 cM (according to the positions employed in this analysis) to about 30.009 cM (according to the MARC marker map) on the bovine chromosome BTA11. The at least one genetic marker is selected from the group of markers shown in Table 33.

TABLE 33

| | Position employed | Relative position (cM) |
|---|---|---|
| Marker on | in analysis | http://www.marc.usda.gov/ |
| BTA11 | (cM) | genome/cattle/cattle.html |
| MAP4K4* | 10.5 | — |
| IL18RA* | 12.3 | — |
| MNB-40 | 16.0 | 19.440 |
| AUP1* | 17.6 | — |
| BM716 | 17.9 | 19.440 |
| DIK2653 | 18.1 | 20.135 |
| BMS2569 | 18.3 | 21.082 |
| BMS2325 | 18.5 | 21.082 |
| BMS1953 | 18.8 | 21.537 |
| DIK4637 | 19.4 | 22.527 |
| UMBTL103 | 21.8 | 23.829 |
| BP38 | 22.6 | 24.617 |
| MNB-70 | 22.8 | 24.617 |
| BM2818 | 26.5 | 30.009 |

In another embodiment of the invention the at least one genetic marker is located on the bovine chromosome BTA11. In one embodiment the at least one genetic marker is located on the bovine chromosome BTA11 in the region flanked by and including the markers IL18RA and UMBTL103. In one embodiment of the present invention, the at least one genetic marker is located in the region from about 12.3 cM (according to the positions employed in this analysis) to about 23.829 cM (according to the MARC marker map) on the bovine chromosome BTA11. The at least one genetic marker is selected from the group of markers shown in Table 34.

TABLE 34

| | Position employed | Relative position (cM) |
|---|---|---|
| Marker on | in analysis | http://www.marc.usda.gov/ |
| BTA11 | (cM) | genome/cattle/cattle.html |
| IL18RA* | 12.3 | — |
| MNB-40 | 16.0 | 19.440 |
| AUP1* | 17.6 | — |
| BM716 | 17.9 | 19.440 |
| DIK2653 | 18.1 | 20.135 |
| BMS2569 | 18.3 | 21.082 |
| BMS2325 | 18.5 | 21.082 |
| BMS1953 | 18.8 | 21.537 |
| DIK4637 | 19.4 | 22.527 |
| UMBTL103 | 21.8 | 23.829 |

In another embodiment of the invention the at least one genetic marker is located on the bovine chromosome BTA11. In one embodiment the at least one genetic marker is located on the bovine chromosome BTA11 in the region flanked by and including the markers MNB-40 and DIK2653. In one embodiment of the present invention, the at least one genetic marker is located in the region from about 19.440 cM to about 20.135 cM on the bovine chromosome BTA11. The at least one genetic marker is selected from the group of markers shown in Table 35.

TABLE 35

| | Position employed | Relative position (cM) |
|---|---|---|
| Marker on | in analysis | http://www.marc.usda.gov/ |
| BTA11 | (cM) | genome/cattle/cattle.html |
| MNB-40 | 16.0 | 19.440 |
| AUP1* | 17.6 | — |
| BM716 | 17.9 | 19.440 |
| DIK2653 | 18.1 | 20.135 |

In another embodiment of the invention the at least one genetic marker is located on the bovine chromosome BTA11. In one embodiment the at least one genetic marker is located on the bovine chromosome BTA11 in the region flanked by and including the markers BM716 and DIK4637. In one embodiment of the present invention, the at least one genetic marker is located in the region from about 19.440 cM to about 22.527 cM on the bovine chromosome BTA11. The at least one genetic marker is selected from the group of markers shown in Table 36.

TABLE 36

| | Position employed | Relative position (cM) |
|---|---|---|
| Marker on | in analysis | http://www.marc.usda.gov/ |
| BTA11 | (cM) | genome/cattle/cattle.html |
| BM716 | 17.9 | 19.440 |
| DIK2653 | 18.1 | 20.135 |
| BMS2569 | 18.3 | 21.082 |
| BMS2325 | 18.5 | 21.082 |
| BMS1953 | 18.8 | 21.537 |
| DIK4637 | 19.4 | 22.527 |

In another embodiment of the invention the at least one genetic marker is located on the bovine chromosome BTA11. In one embodiment the at least one genetic marker is located on the bovine chromosome BTA11 in the region flanked by and including the markers BM716 and BMS2569. In one embodiment of the present invention, the at least one genetic marker is located in the region from about 19.440 cM to about 21.082 cM on the bovine chromosome BTA11. The at least one genetic marker is selected from the group of markers shown in Table 37.

TABLE 37

| | Position employed | Relative position (cM) |
|---|---|---|
| Marker on | in analysis | http://www.marc.usda.gov/ |
| BTA11 | (cM) | genome/cattle/cattle.html |
| BM716 | 17.9 | 19.440 |
| DIK2653 | 18.1 | 20.135 |
| BMS2569 | 18.3 | 21.082 |

In another embodiment of the invention the at least one genetic marker is located on the bovine chromosome BTA11. In one embodiment the at least one genetic marker is located on the bovine chromosome BTA11 in the region flanked by and including the markers BMS2325 and DIK4637. In one embodiment of the present invention, the at least one genetic marker is located in the region from about 21.082 cM to about 22.527 cM on the bovine chromosome BTA11. The at least one genetic marker is selected from the group of markers shown in Table 38.

TABLE 38

| | Position employed | Relative position (cM) |
|---|---|---|
| Marker on | in analysis | http://www.marc.usda.gov/ |
| BTA11 | (cM) | genome/cattle/cattle.html |
| BMS2325 | 18.5 | 21.082 |
| BMS1953 | 18.8 | 21.537 |
| DIK4637 | 19.4 | 22.527 |

In one embodiment of the invention the at least one genetic marker is located on the bovine chromosome BTA11. In one embodiment the at least one genetic marker is located on the bovine chromosome BTA11 in the region flanked by and including the markers IL18RA and AUP1. In one embodiment of the present invention, the at least one genetic marker is located in the region from about 12.3 cM (according to the positions employed in this analysis) to about 17.6 cM (according to the positions employed in this analysis) on the bovine chromosome BTA11. The at least one genetic marker is selected from the group of markers shown in Table 39.

TABLE 39

| | Position employed | Relative position (cM) |
|---|---|---|
| Marker on | in analysis | http://www.marc.usda.gov/ |
| BTA11 | (cM) | genome/cattle/cattle.html |
| IL18RA* | 12.3 | — |
| MNB-40 | 16.0 | 19.440 |
| AUP1* | 17.6 | — |

In one embodiment of the invention the at least one genetic marker is located on the bovine chromosome BTA11. In one embodiment the at least one genetic marker is located on the bovine chromosome BTA11 in the region flanked by and including the markers IL18RA and MNB-40. In one embodiment of the present invention, the at least one genetic marker is located in the region from about 12.3 cM (according to the positions employed in this analysis) to about 19.440 cM (according to the MARC marker map) on the bovine chromosome BTA11. The at least one genetic marker is selected from the group of markers shown in Table 40.

TABLE 40

| | Position employed | Relative position (cM) |
|---|---|---|
| Marker on | in analysis | http://www.marc.usda.gov/ |
| BTA11 | (cM) | genome/cattle/cattle.html |
| IL18RA* | 12.3 | — |
| MNB-40 | 16.0 | 19.440 |

In one embodiment of the invention the at least one genetic marker is located on the bovine chromosome BTA11. In one embodiment the at least one genetic marker is located on the bovine chromosome BTA11 in the region flanked by and including the markers MNB-40 and AUP1. In one embodiment of the present invention, the at least one genetic marker is located in the region from about 19.440 cM (according to the MARC marker map) to about 17.6 cM (according to the positions employed in this analysis) on the bovine chromosome BTA11. The at least one genetic marker is selected from the group of markers shown in Table 41.

TABLE 41

| | Position employed | Relative position (cM) |
|---|---|---|
| Marker on | in analysis | http://www.marc.usda.gov/ |
| BTA11 | (cM) | genome/cattle/cattle.html |
| MNB-40 | 16.0 | 19.440 |
| AUP1* | 17.6 | — |

In one embodiment of the invention the at least one genetic marker is located on the bovine chromosome BTA11. In one embodiment the at least one genetic marker is located on the bovine chromosome BTA11 in the region including the marker IL18RA. In one embodiment of the present invention, the at least one genetic marker is located in the region of about 12.3 cM (according to the positions employed in this analysis) on the bovine chromosome BTA11. The at least one genetic marker is shown in Table 42.

TABLE 42

| | Position employed | Relative position (cM) |
|---|---|---|
| Marker on | in analysis | http://www.marc.usda.gov/ |
| BTA11 | (cM) | genome/cattle/cattle.html |
| IL18RA* | 12.3 | — |

In one embodiment of the invention the at least one genetic marker is located on the bovine chromosome BTA11. In one embodiment the at least one genetic marker is located on the bovine chromosome BTA11 in the region including the marker MNB-40. In one embodiment of the present invention, the at least one genetic marker is located in the region of about 16.0 cM (according to the positions employed in this analysis) on the bovine chromosome BTA11. The at least one genetic marker is shown in Table 43.

TABLE 43

| | Position employed | Relative position (cM) |
|---|---|---|
| Marker on | in analysis | http://www.marc.usda.gov/genome/ |
| BTA11 | (cM) | cattle/cattle.html |
| MNB-40 | 16.0 | 19.440 |

In one embodiment of the invention the at least one genetic marker is located on the bovine chromosome BTA11. In one embodiment the at least one genetic marker is located on the bovine chromosome BTA11 in the region including the marker AUP1.

In one embodiment of the present invention, the at least one genetic marker is located in the region of about 17.6 cM (according to the positions employed in this analysis) on the bovine chromosome BTA11. The at least one genetic marker is shown in Table 44.

TABLE 44

| | Position employed | Relative position (cM) |
|---|---|---|
| Marker on | in analysis | http://www.marc.usda.gov/genome/ |
| BTA11 | (cM) | cattle/cattle.html |
| AUP1* | 17.6 | — |

In one embodiment of the invention the at least one genetic marker is located on the bovine chromosome BTA11. In one embodiment the at least one genetic marker is located on the bovine chromosome BTA11 in the region flanked by and including the markers DIK4637 and UMBTL103. In one embodiment of the present invention, the at least one genetic marker is located in the region from about 22.527 cM to about 23.829 cM (according to the MARC marker map) on the bovine chromosome BTA11. The at least one genetic marker is selected from the group of markers shown in Table 45.

TABLE 45

| | Position employed | Relative position (cM) |
|---|---|---|
| Marker on | in analysis | http://www.marc.usda.gov/genome/ |
| BTA11 | (cM) | cattle/cattle.html |
| DIK4637 | 19.4 | 22.527 |
| UMBTL103 | 21.8 | 23.829 |

In one embodiment of the invention the at least one genetic marker is located on the bovine chromosome BTA11. In one embodiment the at least one genetic marker is located on the bovine chromosome BTA11 in the region including the marker DIK4637. In one embodiment of the present invention, the at least one genetic marker is located in the region of about 22.527 cM (according to the MARC marker map) on the bovine chromosome BTA11. The at least one genetic marker is shown in Table 46.

TABLE 46

| | Position employed | Relative position (cM) |
|---|---|---|
| Marker on | in analysis | http://www.marc.usda.gov/genome/ |
| BTA11 | (cM) | cattle/cattle.html |
| DIK4637 | 19.4 | 22.527 |

In one embodiment of the invention the at least one genetic marker is located on the bovine chromosome BTA11. In one embodiment the at least one genetic marker is located on the bovine chromosome BTA11 in the region including the marker UMBTL103. In one embodiment of the present invention, the at least one genetic marker is located in the region of about 23.829 cM (according to the MARC marker map) on the bovine chromosome BTA11. The at least one genetic marker is shown in Table 47.

TABLE 47

| | Position employed | Relative position (cM) |
|---|---|---|
| Marker on | in analysis | http://www.marc.usda.gov/genome/ |
| BTA11 | (cM) | cattle/cattle.html |
| UMBTL103 | 21.8 | 23.829 |

In another embodiment of the present invention, the at least one genetic marker is a combination of markers, wherein any regions and markers of BTA9 is combined with any regions and markers of BTA11, as described elsewhere herein.

### Primers

The primers that may be used according to the present invention are shown in Table 50. The in Table 50 specified primer pairs may be used individually or in combination with one or more primer pairs of Table 50.

The design of such primers or probes will be apparent to the molecular biologist of ordinary skill. Such primers are of any convenient length such as up to 50 bases, up to 40 bases, more conveniently up to 30 bases in length, such as for example 8-25 or 8-15 bases in length. In general such primers will comprise base sequences entirely complementary to the corresponding wild type or variant locus in the region. However, if required one or more mismatches may be introduced, provided that the discriminatory power of the oligonucleotide probe is not unduly affected. The primers/probes of the invention may carry one or more labels to facilitate detection.

In one embodiment, the primers and/or probes are capable of hybridizing to and/or amplifying a subsequence hybridizing to a single nucleotide polymorphism containing the sequence delineated by the markers as shown herein.

The primer nucleotide sequences of the invention further include: (a) any nucleotide sequence that hybridizes to a nucleic acid molecule comprising a genetic marker sequence or its complementary sequence or RNA products under stringent conditions, e.g., hybridization to filter-bound DNA in 6× sodium chloride/sodium citrate (SSC) at about 45° C. followed by one or more washes in 0.2×SSC/0.1% Sodium Dodecyl Sulfate (SDS) at about 50-65° C., or (b) under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel F. M. et al., eds., 1989, Current Protocols in Molecular Biology, Vol. I, Green Publishing Associates, Inc., and John Wiley & sons, Inc., New York, at pp. 6.3.1-6.3.6 and 2.10.3). Preferably the nucleic acid molecule that hybridizes to the nucleotide sequence of (a) and (b), above, is one that comprises the complement of a nucleic acid molecule of the genomic DNA comprising the genetic marker sequence or a complementary sequence or RNA product thereof.

Among the nucleic acid molecules of the invention are deoxyoligonucleotides (“oligos”) which hybridize under highly stringent or stringent conditions to the nucleic acid molecules described above. In general, for probes between 14 and 70 nucleotides in length the melting temperature (TM) is calculated using the formula:

Tm(° C.)=81.5+16.6(log [monovalent cations(molar)])+0.41(% G+C)−(500/N)

where N is the length of the probe. If the hybridization is carried out in a solution containing formamide, the melting temperature is calculated using the equation Tm(° C.)=81.5+16.6(log [monovalent cations (molar)])+0.41 (% G+C)−(0.61% formamide)−(500/N) where N is the length of the probe. In general, hybridization is carried out at about 20-25 degrees below Tm (for DNA-DNA hybrids) or 10-15 degrees below Tm (for RNA-DNA hybrids).

Exemplary highly stringent conditions may refer, e.g., to washing in 6×SSC/0.05% sodium pyrophosphate at 37° C. (for about 14-base oligos), 48° C. (for about 17-base oligos), 55° C. (for about 20-base oligos), and 60° C. (for about 23-base oligos).

Accordingly, the invention further provides nucleotide primers or probes which detect the polymorphisms of the invention. The assessment may be conducted by means of at least one nucleic acid primer or probe, such as a primer or probe of DNA, RNA or a nucleic acid analogue such as peptide nucleic acid (PNA) or locked nucleic acid (LNA).

According to one aspect of the present invention there is provided an allele-specific oligonucleotide probe capable of detecting a polymorphism at one or more of positions in the delineated regions.

The allele-specific oligonucleotide probe is preferably 5-50 nucleotides, more preferably about 5-35 nucleotides, more preferably about 5-30 nucleotides, more preferably at least 9 nucleotides.

### Determination of Linkage

In order to detect if the genetic marker is present in the genetic material, standard methods well known to persons skilled in the art may be applied, e.g. by the use of nucleic acid amplification. In order to determine if the genetic marker is genetically linked to mastitis resistance traits, a permutation test can be applied (Doerge and Churchill, 1996), or the Piepho-method can be applied (Piepho, 2001). The principle of the permutation test is well described by Doerge and Churchill (1996), whereas the Piepho-method is well described by Piepho (2001). Significant linkage in the within family analysis using the regression method, a 10000 permutations were made using the permutation test (Doerge and Churchill, 1996). A threshold at the 5% chromosome wide level was considered to be significant evidence for linkage between the genetic marker and the mastitis resistance and somatic cell count traits. In addition, the QTL was confirmed in different sire families. For the across family analysis and multi-trait analysis with the variance component method, the Piepho-method was used to determine the significance level (Piepho, 2001). A threshold at the 5% chromosome wide level was considered to be significant evidence for linkage between the genetic marker and the mastitis resistance and somatic cell count traits.

### Kit

Another aspect of the present invention relates to diagnostic kit for use in detecting the presence or absence in a bovine subject of at least one genetic marker associated with resistance to mastitis, comprising at least one oligonucleotide sequence and combinations thereof, wherein the nucleotide sequences are selected from any of SEQ ID NO.: 1 to SEQ ID NO.: 192 and/or any combination thereof.

Genotyping of a bovine subject in order to establish the genetic determinants of resistance to mastitis for that subject according to the present invention can be based on the analysis of DNA and/or RNA. One example is genomic DNA which can be provided using standard DNA extraction methods as described herein. The genomic DNA may be isolated and amplified using standard techniques such as the polymerase chain reaction using oligonucleotide primers corresponding (complementary) to the polymorphic marker regions. Additional steps of purifying the DNA prior to amplification reaction may be included. Thus, a diagnostic kit for establishing mastitis resistance and somatic cell count characteristics comprises, in a separate packing, at least one oligonucleotide sequence selected from the group of sequences shown in table xx and any combinations thereof.

### EXAMPLES

### Animals

The animal material consists of a grand daughter design with 39 paternal sire families with a total number of offspring tested sons was 1513 from four dairy cattle breeds namely Danish Holstein (DH) and Danish Red (DR), Finnish Ayrshire (FA) and Swedish Red and White (SRB). These 39 families consist of 5 DH, 9 DR, 11 FA and 14 SRB grandsire families. The number of sons per grandsire ranged from 16 to 161, with an average family size of 38.8.

### Purification of Genomic DNA

Genomic DNA was purified from semen according to the following protocol:

After thawing the semen-straw, both ends of the straw were cut away with a pair of scissors and the content of semen transferred to a 1.5 ml eppendorf tube. 1 ml of 0.9% NaCl was used to flush the straw into the tube. The tube was then centrifuged for 5 minutes at 2000 rpm, followed by removal of the supernatant. This washing step was repeated twice.

Then 300111 buffer S (10 mM Tris HCl pH 8, 100 mM NaCl, 10 mM EDTA pH 8; 0.5% SDS), 20 μl 1 M DTT and 20 μl pronase (20 mg/ml) (Boehringer) are added to the tube. After mixing the tubes are incubated over night with slow rotation where after 180 μl saturated NaCl is added followed by vigorous agitation for 15 seconds. The tube is the centrifuged for 15 minutes at 11000 rpm. 0.4 ml of the supernatant is transferred to a 2 ml tube and 1 ml of 96% ethanol is added, mixing is achieved by slow rotation of the tube. The tube is then centrifuged for 10 minutes at 11000 rpm. Remove the supernatant by pouring away the liquid, wash the pellet with 70% ethanol (0.2 ml) and centrifuge again for 10 minutes at 11000 rpm. Pour away the ethanol, dry the pellet and resuspend in 0.5 ml of TE-buffer) for 30 minutes at 55° C.

### Amplification Procedures

PCR reactions were run in a volume of 8111 using TEMPase (GeneChoice) polymerase and reaction buffer I as provided by the supplier (GeneChoice). Usually 5 different markers are included in each multiplex PCR. 1 μl DNA, 0.1 μl TEMPase enzyme, 0.2 mM dNTPs, 1.2 mM MgCl2, 0.3 μM each primer.

The PCR mixtures were subjected to initial denaturation at 94° C. for 15 min (for TEMPase). Subsequently, the samples were cycled for 10 cycles with touchdown, i.e. the temperature is lowered 1° C. at each cycle (denaturation at 94° C. 30″, annealing at 67° C. 45″, elongation 72° C. 30″), after which the samples were cycled for 20 cycles with normal PCR conditions (denaturation at 94° C. 30″, annealing at 58° C. 45″, elongation 72° C. 30) PCR cycling was terminated by 1 cycle at 72° C. 30′ and the PCR machine was programmed to cooling down the samples at 4° C. for ‘ever’.

The nucleotide sequence of the primers used for detecting the markers is shown in Table 50. The nucleotide sequence is listed from the 5′ end.

TABLE 50

| | | |
|---|---|---|
| Marker | Forward Primer F | |
| name | Reverse Primer R | SEQ ID NO.: |

| | | |
|---|---|---|
| BTA9: | | |
| BMS2151 | F AACGGCTTTCACTTTCTTGC | SEQ ID NO.: 1 |
| | R CTGGGTGAACAAATGGGC | SEQ ID NO.: 2 |
| ETH225 | F GATCACCTTGCCACTATTTCCT | SEQ ID NO.: 3 |
| | R ACATGACAGCCAGCTGCTACT | SEQ ID NO.: 4 |
| BM2504 | F CAGCTTTCCATCCCCTTTC | SEQ ID NO.: 5 |
| | R CTCCCATCCCAAACACAGAC | SEQ ID NO.: 6 |
| DIK2892 | F TTGACCCTGAAAGATGTCCA | SEQ ID NO.: 7 |
| | R CACGGTTTATCAGCTTGGGTA | SEQ ID NO.: 8 |
| DIK 3002 | F AAATGGAGGTAATGAAATAAAATA | SEQ ID NO.: 9 |
| | R CAAACCCATGGACTGTAACCT | SEQ ID NO.: 10 |
| DIK 3003 | F ACTTTCAGTTTTGGGCTGAC | SEQ ID NO.: 11 |
| | R TGTCACTAGGTAAATTGGTG | SEQ ID NO.: 12 |
| RM216 | F TTCTGCAATGTTGAGCTTCAAG | SEQ ID NO.: 13 |
| | R GATCTGAAAAAGAAATGAATAGA | SEQ ID NO.: 14 |
| BMS817 | F TGGGAAAGTTGGCAAATG | SEQ ID NO.: 15 |
| | R TTGTGATACCTGAAATGGTCAA | SEQ ID NO.: 16 |
| BMS555 | F GGAAAGAGTAGGTGATTCCCTG | SEQ ID NO.: 17 |
| | R ATTTAATTGTCATCCCAGGTGA | SEQ ID NO.: 18 |
| lama4 | F TTAAAGCAATTTAGGGAGCTTA | SEQ ID NO.: 19 |
| | R CTAGTATCTAAAATGAACAGAA | SEQ ID NO.: 20 |
| DIK 5142 | F TGGGTAAGTGGGAAAGGATG | SEQ ID NO.: 21 |
| | R CTCAGCCAGGTTGTCCTCTC | SEQ ID NO.: 22 |
| slc16a10 | F CAGGTACACAGTAAAGACAGA | SEQ ID NO.: 23 |
| | R CTGCTTTGGGGGCACAGTCA | SEQ ID NO.: 24 |
| DIK 4268 | F ATAAGGGTGCACTGGCAGAA | SEQ ID NO.: 25 |
| | R GCAGTCCAGGGGATTGTAAA | SEQ ID NO.: 26 |
| DIK 4950 | F AGTGCCTGGCAGGTATTGAA | SEQ ID NO.: 27 |
| | R CCTCGGTTTCCCAATCATTA | SEQ ID NO.: 28 |
| CSSM025 | F GTAGTTATCAAAATAAGAATGCTT | SEQ ID NO.: 29 |
| | R TATGTTTTCCTTTTGGTTGAATAG | SEQ ID NO.: 30 |
| DIK 2810 | F TCTGAAACCTGGAGGAGGAG | SEQ ID NO.: 31 |
| | R GAAACTTCCACCACCCTCAA | SEQ ID NO.: 32 |
| DIK 5364 | F CCTCTGAAACCCCAGACTTG | SEQ ID NO.: 33 |
| | R AAAAACCCAAAACAACACACAA | SEQ ID NO.: 34 |
| DIK 2741 | F TCCCCAAATTCTGATGACTCT | SEQ ID NO.: 35 |
| | R TCAGCCCTTAAAACGTAAGCA | SEQ ID NO.: 36 |
| TGLA261 | F TCAAATCTCATTCTCTCCAGAAGGC | SEQ ID NO.: 37 |
| | R CCAACTTATATTAGGCACAATGTCC | SEQ ID NO.: 38 |
| ILSTS013 | F CTTGATCCTTATAGAACTGG | SEQ ID NO.: 39 |
| | R ACACAAAATCAGATCAGTGG | SEQ ID NO.: 40 |
| UWCA9 | F CCTTCTCTGAATTTTTGTTGAAAGC | SEQ ID NO.: 41 |
| | R GGACAGAAGTGAGTGACTGAGA | SEQ ID NO.: 42 |
| BMS1148 | F TTAAATGGGACCAGATAAATAGGA | SEQ ID NO.: 43 |
| | R AAATGAGAACCAGATAAGCCTAAA | SEQ ID NO.: 44 |
| DIK 4912 | F AAGAAGTAGAGCGGGGGAAG | SEQ ID NO.: 45 |
| | R GAATGCCAAGCATCCCTTAC | SEQ ID NO.: 46 |
| DIK 5130 | F TTGCAGTGATCTCTGCTAAAGTG | SEQ ID NO.: 47 |
| | R TCTCCCCACAACATCATTCA | SEQ ID NO.: 48 |
| DIK 2303 | F GGAAAGACAAGAGGGTGCTG | SEQ ID NO.: 49 |
| | R TGTTGCAAAAAGCAAATTTCA | SEQ ID NO.: 50 |
| DIK 4720 | F CATGATATTTACCCTGTGTGTGC | SEQ ID NO.: 51 |
| | R GAGGAGCTGGAGGGCTAAAG | SEQ ID NO.: 52 |
| BM4204 | F GGGTAGGAGCTTTTGTAGGTG | SEQ ID NO.: 53 |
| | R GCCATCACCCTTCTCTTATATG | SEQ ID NO.: 54 |
| DIK 4926 | F ATGACTCCTGGAGCAGAACC | SEQ ID NO.: 55 |
| | R GAAGAGTAAGCTGTATTTTTCATGC | SEQ ID NO.: 56 |
| BMS1909 | F ACTTGTTAGGAGGGCTATTGTTAA | SEQ ID NO.: 57 |
| | R CCACATACACCACCAACATTAA | SEQ ID NO.: 58 |
| BMS1290 | F TTGGCACTTACTACCTCATATGTT | SEQ ID NO.: 59 |
| | R TTTTCTGGATGTTGAGCCTATT | SEQ ID NO.: 60 |
| TGLA73 | F GAGAATCACCTAGAGAGGCA | SEQ ID NO.: 61 |
| | R CTTTCTCTTTAAATTCTATATGGT | SEQ ID NO.: 62 |
| BMS2753 | F TCAAAAAGTTGGACATGACTGA | SEQ ID NO.: 63 |
| | R AGGTTTTCAAATGAGAGACTTTTC | SEQ ID NO.: 64 |
| TNF | F GGAGGGTGTGCTTGAAAGAG | SEQ ID NO.: 65 |
| | R GCTGGCGTTCTCTCTCGTAT | SEQ ID NO.: 66 |
| BMS1724 | F GACTTGCCCCAATCCTACTG | SEQ ID NO.: 67 |
| | R ATTTCAGGTTTGTTGGTTCCC | SEQ ID NO.: 68 |
| DIK 2145 | F TGGTGCTCTGGGAACATAGAC | SEQ ID NO.: 69 |
| | R ATCACAGTGGCCTGAACACA | SEQ ID NO.: 70 |
| BM7209 | F TTTTCTGCTCATGCTTCAGTG | SEQ ID NO.: 71 |
| | R GCAGGCTATAGTCCATGACATC | SEQ ID NO.: 72 |
| SLU2 | F GGGTTCTGTTTGCTTTTCTTC | SEQ ID NO.: 73 |
| | R CTAGCACTGGCAGGTAGATTCT | SEQ ID NO.: 74 |
| C6orf93 | F CTCGGTGATGTTTTTGCTGA | SEQ ID NO.: 75 |
| | R CGCCCCAGCTCTTTCTAGTT | SEQ ID NO.: 76 |
| DIK 4986 | F GGGATGAACATTGAGGGTTG | SEQ ID NO.: 77 |
| | R CATGATCAAGATGGGGGAAG | SEQ ID NO.: 78 |
| Mm12e6 | F CAAGACAGGTGTTTCAATCT | SEQ ID NO.: 79 |
| | R ATCGACTCTGGGGGATGATGT | SEQ ID NO.: 80 |
| PEX3 | F TTTTGCGAGTCCAGTTAAACA | SEQ ID NO.: 81 |
| | R GGAAAAGCCAGAGCAAAATG | SEQ ID NO.: 82 |
| DEADC1 | F TTCCTAGGCCTGTGCTCATT | SEQ ID NO.: 83 |
| | R TGGACCAGGCATAAGGATTT | SEQ ID NO.: 84 |
| BMS2251 | F AACGGCTTTCACTTTCTTGC | SEQ ID NO.: 85 |
| | R CTGGGTGAACAAATGGGC | SEQ ID NO.: 86 |
| EPM2A | F GCGGCC GCGTTGAGAG | SEQ ID NO.: 87 |
| | R TTCCAC TTT ATG ATG AGC AGG TTC | SEQ ID NO.: 88 |
| BM7234 | F TTCACTGATTGTCATTCCCTAGA | SEQ ID NO.: 89 |
| | R TAAGCAAATAAATGGTGCTAGTCA | SEQ ID NO.: 90 |
| BM4208 | F TCAGTACACTGGCCACCATG | SEQ ID NO.: 91 |
| | R CACTGCATGCTTTTCCAAAC | SEQ ID NO.: 92 |
| BMS2819 | F GCTCACAGGTTCTGAGGACTC | SEQ ID NO.: 93 |
| | R AACTTGAAGAAGGAATGCTGAG | SEQ ID NO.: 94 |
| INRA144 | F TCGGTGTGGGAGGTGACTACAT | SEQ ID NO.: 95 |
| | R TGCTGGTGGGCTCCGTCACC | SEQ ID NO.: 96 |
| INRA084 | F CTAAAGCTTTCCTCCATCTC | SEQ ID NO.: 97 |
| | R CCTGGTGATGTTTGGATGTC | SEQ ID NO.: 98 |
| rgsl7 | F CATGAAACACAAACATAAATGGGA | SEQ ID NO.: 99 |
| | R GGGACCAAAAATACATCACAGTA | SEQ ID NO.: 100 |
| ESR1 | F GCTGCTGGAGATGCTGGAT | SEQ ID NO.: 101 |
| | R TGATTCACGTCCTCTGGAGGT | SEQ ID NO.: 102 |
| BMS2295 | F GCTCTGGTGACCCAGGTG | SEQ ID NO.: 103 |
| | R CTGGCAGGAGATGAGAGGAG | SEQ ID NO.: 104 |
| BM3215 | F TGCATCAACTAAGCCACACTG | SEQ ID NO.: 105 |
| | R TTACTCGCTGGTTTTCTGGG | SEQ ID NO.: 106 |
| bvil203 | F CGAGTTCGAGGCCATGTGAA | SEQ ID NO.: 107 |
| | R CGGAGCAGGGAGAGGGT | SEQ ID NO.: 108 |
| Aridlb | F CTGTTCTATTCCCTATACTG | SEQ ID NO.: 109 |
| | R ATTATCATGCATACACTTTGA | SEQ ID NO.: 110 |
| Plg | F CAGGGTGACAGCGGCGGGCC | SEQ ID NO.: 111 |
| | R GAAGTACCGAGTTTATTTTCAACAAAT | SEQ ID NO.: 112 |
| Igfsnp123 | F CAAGACCGGCCTGAGCTACAAGAG | SEQ ID NO.: 113 |
| | R GTGCGGTGGATGAGTGGGGACAG | SEQ ID NO.: 114 |
| BMS1943 | F ATCAGTCGTTCCCAGAATGTC | SEQ ID NO.: 115 |
| | R TTGATATCCTCTCTGTCAAGCC | SEQ ID NO.: 116 |
| BMS1967 | F GGGCAGATGTGAGTAATTTTCC | SEQ ID NO.: 117 |
| | R AACTGAGCTGTATGGTGGACG | SEQ ID NO.: 118 |
| BTA11: | | |
| HELMTT43 | F GGTTACAGTCCATGAGTTTGCAAAG | SEQ ID NO.: 119 |
| | R ACAGAGGTGGGGTAGACTTTT | SEQ ID NO.: 120 |
| ZAP70 | F GGAGCTACGGAGTCACCATGT | SEQ ID NO.: 121 |
| | R GTAGGTCCAGCAATCGCTCAT | SEQ ID NO.: 122 |
| MAP4K4 | F CAAAGAGTGGGTCTCAACATGAATC | SEQ ID NO.: 123 |
| | R GGGCTGGGCCTGCTC | SEQ ID NO.: 124 |
| IL18RA | F CAGAAGTCTTGCCTGGGAAGTC | SEQ ID NO.: 125 |
| | R CCGTGTCTGCCTCTTGTGA | SEQ ID NO.: 126 |
| MNB-40 | F CAGCCTCCTTCATACTCCTTCT | SEQ ID NO.: 127 |
| | R GGGGAAGGGAGCAGATTGTA | SEQ ID NO.: 128 |
| AUP1 | F CCCTGTCCTGACGTCTGTTT | SEQ ID NO.: 129 |
| | R CACAACCAAGGGAAAAGGAA | SEQ ID NO.: 130 |
| BM716 | F AGTACTTGGCTTGCTTTGCTC | SEQ ID NO.: 131 |
| | R TTAAATTTCCATCTCACCCTGG | SEQ ID NO.: 132 |
| DIK2653 | F ATGGCCGTCCATTCAGATAC | SEQ ID NO.: 133 |
| | R CCTCCCTGTGGTTTATGGAA | SEQ ID NO.: 134 |
| BMS2569 | F AGAGAGGCCAAAGCTGGG | SEQ ID NO.: 135 |
| | R TTTCCTTGGGCTTCAGGAG | SEQ ID NO.: 136 |
| BMS2325 | F TCCATCTTGCAGAAGTGTGC | SEQ ID NO.: 137 |
| | R AGGGCCAGGAATGCTAGTG | SEQ ID NO.: 138 |
| BMS1953 | F TGCTGTAGGAGAAAATAAAGCAG | SEQ ID NO.: 139 |
| | R TTTGCTGAGAGGACTTTGAGA | SEQ ID NO.: 140 |
| DIK4637 | F TGTGCTCTAAAGCTTGACCTG | SEQ ID NO.: 141 |
| | R TCAGCTGGTTGAGGGTTCTC | SEQ ID NO.: 142 |
| UMBTL103 | F TCTCGTTCATAGGTGGCATCT | SEQ ID NO.: 143 |
| | R TTGGATGGCATCACTGACTTG | SEQ ID NO.: 144 |
| BP38 | F CCAAATGATGGTTCAAGTTTG | SEQ ID NO.: 145 |
| | R GCTCATGATAAAGGGAATTCAG | SEQ ID NO.: 146 |
| MNB-70 | F TAATGAGCAGACCCACACAG | SEQ ID NO.: 147 |
| | R ACCATTGGCTCTCCTAGGTC | SEQ ID NO.: 148 |
| BM2818 | F TTCTGTGGTTGAAGAGTGTTCC | SEQ ID NO.: 149 |
| | R CAATGGCTAAGAGGTCCAGTG | SEQ ID NO.: 150 |
| BM304 | F CTGGTGTTCCTTTCATATCAACC | SEQ ID NO.: 151 |
| | R GGCACGTACTAACCTGTAAAACC | SEQ ID NO.: 152 |
| INRA177 | F TCCAAAAGTTTCGTGACATATTG | SEQ ID NO.: 153 |
| | R CACCAGGCTTCTCTGTTGAA | SEQ ID NO.: 154 |
| UMBTL20 | F TTCCATGTCACAGATAGCCTC | SEQ ID NO.: 155 |
| | R ACATTATCACAAGACACCAGC | SEQ ID NO.: 156 |
| RM96 | F TCGCAAAAAGTTGGACAAGACT | SEQ ID NO.: 157 |
| | R TTAGCAGGGTGCCTGACACTT | SEQ ID NO.: 158 |
| INRA131 | F GGTAAAATGCTGCAAAACACAG | SEQ ID NO.: 159 |
| | R TGACTGTATAGACTGAAGCAAC | SEQ ID NO.: 160 |
| BM7169 | F TGGTATGTAGTTACAGCAGCCC | SEQ ID NO.: 161 |
| | R CCATTGAAACAGACATGAATGC | SEQ ID NO.: 162 |
| BMS1716 | F GTGGGTTGGAGAGGTACAAG | SEQ ID NO.: 163 |
| | R AGAAATGGCCTTGAGAAAGAG | SEQ ID NO.: 164 |
| BM6445 | F GTGTCTGTCAAAAGATGAATGG | SEQ ID NO.: 165 |
| | R GACAACTGCTTCTCGTTGGG | SEQ ID NO.: 166 |
| CD8B | F GAAGTTGACTGTGCATGGAAATCC | SEQ ID NO.: 167 |
| | R GGCAGGCTTCACATTTTGGA | SEQ ID NO.: 168 |
| MB110 | F ACACATACACACACACGCACA | SEQ ID NO.: 169 |
| | R TGGCTGCTCAAAAAATAGCA | SEQ ID NO.: 170 |
| MS2177 | F TTTGAAGGAGTAAGCACTCTGT | SEQ ID NO.: 171 |
| | R CAGACACAACTGAAGCAACTC | SEQ ID NO.: 172 |
| HELMTT44 | F CACTTAGCCACCTGAAATAGAT | SEQ ID NO.: 173 |
| | R AGCAACTGCCACTTCACTTC | SEQ ID NO.: 174 |
| DIK5170 | F TTTGGACTTGCCAAACCTC | SEQ ID NO.: 175 |
| | R TCAGAGCAACAGAACTAATAAGA | SEQ ID NO.: 176 |
| RM150 | F GAACAGTGGTTACCTGTCTGTC | SEQ ID NO.: 177 |
| | R CTGCCTAACCTTCCTGGCGTC | SEQ ID NO.: 178 |
| TGLA58 | F TTCTAGTCTCCAGCCTCGTCC | SEQ ID NO.: 179 |
| | R GTTGGCTCCAAGAGCAAGTC | SEQ ID NO.: 180 |
| TGLA340 | F GAGGCGTTCACCAACAGTTCACTGA | SEQ ID NO.: 181 |
| | R GATTCCACAGTGCCAGACCCAAGCC | SEQ ID NO.: 182 |
| BM8118 | F TCCTACTTTTGCATTCCAGTCC | SEQ ID NO.: 183 |
| | R ACCACTAAAGTCAAAGAAGCCG | SEQ ID NO.: 184 |
| BMS2047 | F ACTATGGACATTTGGGGCAG | SEQ ID NO.: 185 |
| | R AGTAGGTGGAGATCAAGGATGC | SEQ ID NO.: 186 |
| BMS1048 | F GTTTGATACTATGTCCCTTTGTGTG | SEQ ID NO.: 187 |
| | R GAGTAGCTGCCCCTGTTCTC | SEQ ID NO.: 188 |
| BMS989 | F TTTGAGAACTTTTGTTTCTGAGC | SEQ ID NO.: 189 |
| | R TTATTTTGCTTTTCTGATTTTGTG | SEQ ID NO.: 190 |
| BM3501 | F CCAACGGGTTAAAAGCACTG | SEQ ID NO.: 191 |
| | R TTCCTGTTCCTTCCTCATCTG | SEQ ID NO.: 192 |

### Markers and Map

For BTA9 in the present study 45 microsatellite markers were chosen from the website of the Meat Animal Research Center (www.marc.usda.gov/genome/genome.html). As BTA9 is orthologous to HSA6q, 28 published genes and ESTs were chosen along HSA6q (Ctgf, Vip, Vil2, Rgs17, Ros1, Slc16a10, Oprm, igf2r, Esr1, Deadc1, Pex3, C6orf93, Ifngr1, Shprh, Epm2a, AK094944, AK094379, Utrn, Tnf, plg, arid1b, lama4, hivep2, C6orf055, CITED2, RP1-172K10, AIG1, GRM) for SNPs and microsatellites screening. Eight of new microsatellite markers identified in the present study and 29 SNPs were also genotyped across the pedigree in order to create a dense map of BTA9 by linkage analysis.

Out of total 37 markers in the linkage map of BTA 1, in the present study 30 microsatellite markers were chosen from the website of the Meat Animal Research Centre (http://www.marc.usda.gov/genome/cattle/cattle.html).

### Radiation Hybrid (RH) Panel Information

Specific primer pairs were designed from the bovine sequences to map the genes and microsatellites including MARC microsatellites. Along chromosome 9, a total of more than 120 markers were used on the cattle RH panel. 65 genes and 34 microsatellites showed a successful amplification, bands with the appropriate size on the bovine DNA and no amplification on hamster DNA. They were typed on the 3000-rad panel Roslin/Cambridge bovine RH panel (Williams et al. 2002). PCR amplifications were performed in a total volume of 20 gi containing 25 ng of the RH cell line DNA, 0.5 μM of each primer, 200 μM dNTPs, 3 mM MgCl₂, 0.5 U of Taq polymerase (BIOLine). The reaction conditions were a touch-down starting with 94° C. for 3 min followed by 40 cycles of 93° C. for 30s, 65-45° C. touch-down for 30 s, decreasing 0.5° C. per cycle, and 72° C. for 1 min, with a final extension step of 72° C. for 5 min. PCR reactions were electrophoresed: 10 μl of the PCR product were loaded in ethidium bromide stained mini-gels (2.5% agarose) and the presence or absence of amplicons were scored by two independent observers. Where there were several discrepancies between the patterns from the duplicates or between the scores from the different observers, PCR reactions and gels were repeated. Markers were discarded when the results for several hybrids remained ambiguous.

Markers were assigned to the bovine chromosome by carrying out 2-point linkage analysis using RHMAPPER (Soderlund et al., 1998) against markers with known assignments that had been previously typed on the bovine WGRH panel (Williams et al., 2002). RH map was then constructed using the Carthegene software (Schiex., 2002) as described by Williams et al (2002). On bovine chromosome 9, we have information from a radiation hybrid map with 150 markers.

Marker order and map distances were estimated using CRIMAP 2.4 software (Green et al. 1990). To construct our linkage map we began by placing the microsatellite markers following the MARC map order. Next a BUILD option of CRIMAP was run to place the remaining markers, the new microsatellite and SNP markers have been inserted at the position with the highest likelihood. MARC (www.marc.usda.gov/genome/genome.html), Ensembl (http://www.ensembl.org/Bos_taurus/index.html) and radiation hybrid (RH) information have been taken into account to reconsider that emplacement of the makers. The final linkage map used of the QTL mapping of BTA9 according to the present invention includes 59 markers as listed in table 51.

The final linkage map used of the QTL mapping of BTA11 according to the present invention includes 37 markers as listed in table 52.

The following tables show markers used for the relevant QTL. Any additional information on the markers can be found on ‘http://www.marc.usda.gov/’, http://www.ensembl.org/Bos_taurus/index.html and ‘http://www.ncbi.nih.gov/’.

TABLE 51

| | Position employed | Relative position (cM) |
|---|---|---|
| Marker on BTA9 | in analysis (cM) | http://www.marc.usda.gov/ |
| BMS2151 | 0 | 4.892 |
| ETH225 | 7.4 | 12.754 |
| BM2504 | 25.3 | 30.92 |
| DIK2892 | 25.35 | 30.92 |
| DIK 3002 | 32.7 | 36.542 |
| DIK 3003 | 32.75 | 36.542 |
| RM216 | 32.8 | 37.087 |
| BMS817 | 36.6 | 42.489 |
| BMS555 | 37.4 | 43.818 |
| lama4* | 37.6 | — |
| DIK 5142 | 37.75 | 43.818 |
| slc16a10* | 37.78 | — |
| DIK 4268 | 37.81 | 45.152 |
| DIK 4950 | 37.84 | 45.152 |
| CSSM025 | 37.87 | 45.739 |
| DIK 2810 | 37.9 | 45.739 |
| DIK 5364 | 38.2 | 45.739 |
| DIK 2741 | 39.75 | 49.659 |
| TGLA261 | 39.8 | 49.659 |
| ILSTS013* | 39.85 | — |
| UWCA9 | 39.9 | 49.996 |
| BMS1148 | 39.95 | 50.923 |
| DIK 4912 | 42.45 | 51.855 |
| DIK 5130 | 42.5 | 52.296 |
| DIK 2303 | 42.55 | 52.352 |
| DIK 4720 | 43.3 | 53.966 |
| BM4204 | 45.1 | 55.414 |
| DIK 4926 | 47.6 | 57.088 |
| BMS1909 | 49.8 | 59.516 |
| BMS1290 | 53.8 | 64.935 |
| TGLA73 | 63.3 | 77.554 |
| BMS2753 | 65.4 | 79.249 |
| TNF* | 65.45 | — |
| BMS1724 | 67.15 | 80.265 |
| DIK 2145 | 67.2 | 80.265 |
| BM7209 | 67.6 | 81.569 |
| SLU2 | 68.8 | — |
| C6orf93* | 69.35 | — |
| DIK 4986 | 69.4 | 84.258 |
| mm12e6* | 69.45 | 84.258 |
| PEX3* | 69.5 | — |
| DEADC1 | 69.55 | — |
| BMS2251 | 71.3 | 86.58 |
| EPM2A* | 72.1 | |
| BM7234 | 72.3 | 88.136 |
| BM4208 | 73.9 | 90.69 |
| BMS2819 | 73.95 | 90.98 |
| INRA144 | 74.2 | 90.98 |
| INRA084 | 74.5 | 90.98 |
| rgs17* | 79.8 | — |
| ESR1* | 79.85 | — |
| BMS2295 | 82.2 | 98.646 |
| BM3215 | 83.2 | 101.647 |
| bvil203* | 86.5 | — |
| Aridlb* | 86.6 | — |
| Plg* | 89.4 | — |
| igfsnp123* | 89.45 | — |
| BMS1943 | 92.5 | 103.708 |
| BMS1967 | 97.7 | 109.287 |

*these markers are not listed in the MARC marker map of BTA9.

TABLE 52

| | Position employed | Relative position (cM) |
|---|---|---|
| Marker on BTA11 | in analysis (cM) | http://www.marc.usda.gov |
| HELMTT43 | 0.0 | 2.249 |
| ZAP70* | 54 | — |
| MAP4K4* | 10.5 | — |
| IL18RA* | 12.3 | — |
| MNB-40 | 16.0 | 19.440 |
| AUP1* | 17.6 | — |
| BM716 | 17.9 | 19.440 |
| DIK2653 | 18.1 | 20.135 |
| BMS2569 | 18.3 | 21.082 |
| BMS2325 | 18.5 | 21.082 |
| BMS1953 | 18.8 | 21.537 |
| DIK4637 | 19.4 | 22.527 |
| UMBTL103 | 21.8 | 23.829 |
| BP38 | 22.6 | 24.617 |
| MNB-70 | 22.8 | 24.617 |
| BM2818 | 26.5 | 30.009 |
| BM304 | 30.0 | 33.597 |
| INRA177 | 32.5 | 35.098 |
| UMBTL20 | 32.7 | 34.802 |
| RM96* | 38.0 | — |
| INRA131 | 43.6 | 47.289 |
| BM7169 | 46.8 | 50.312 |
| BMS1716 | 50.2 | 54.581 |
| BM6445 | 55.1 | 61.570 |
| CD8B* | 56.9 | — |
| MB110 | 59.6 | 68.679 |
| MS2177 | 61.0 | 69.415 |
| HELMTT44* | 61.2 | — |
| DIK5170 | 61.4 | 70.143 |
| RM150 | 61.8 | 70.143 |
| TGLA58 | 63.1 | 73.136 |
| TGLA340 | 65.8 | 75.208 |
| BM8118 | 67.2 | 77.063 |
| BMS2047 | 68.8 | 78.457 |
| BMS1048 | 69.5 | 81.065 |
| BMS989 | 78.9 | 92.179 |
| BM3501 | 85.2 | 97.223 |

*these markers are not listed in the MARC marker map of BTA11.

### Phenotypic Data

Daughters of bulls were scored for mastitis resistance and SCS. Estimated breeding values (EBV) for traits of sons were calculated using a single trait Best Linear Unbiased Prediction (BLUP) animal model ignoring family structure. These EBVs were used in the QTL analysis. The daughter registrations used in the individual traits were:

Clinical mastitis in Denmark: Treated cases of clinical mastitis in the period −5 to 50 days after 1^{st} calving.

Clinical mastitis in Sweden and Finland: Treated cases of clinical mastitis in the period −7 to 150 days after 1^{st} calving.

SCS in Denmark: Mean SCS in period 10-180 days after 1^{st} calving.

SCS in Sweden: Mean SCS in period 10-150 days after 1^{st} calving.

SCS in Finland: Mean SCS in period 10-305 days after 1^{st} calving.

### Example 1

### BTA9

### Statistical Analysis

A number of statistical methods as described below were used in the determination of genetic markers associated or linked to mastitis and thus mastitis resistance.

### QTL Analysis

Linkage analysis (LA) is used to identify QTL by typing genetic markers in families to chromosome regions that are associated with disease or trait values within pedigrees more often than are expected by chance. Such linked regions are more likely to contain a causal genetic variant. The data was analysed with a series of models. Initially, a single trait model using a multipoint regression approach for all traits were analysed within family. Chromosomes with significant effects within families were analysed with the variance component method to validate QTL found across families and for characterization of QTL.

### Regression Analysis

Population allele frequencies at the markers were estimated using an EM-algorithm. Allele frequencies were subsequently assumed known without error. Phase in the sires was determined based on offspring marker types. Subsequently this phase was assumed known without error. Segregation probabilities at each map position were calculated using information from all markers on the chromosome simultaneously using Haldane's mapping function (Haldane, 1919). Phenotypes were regressed onto the segregation probabilities. Significance thresholds were calculated using permutation tests (Churchill and Doerge, 1994).

### Variance Component Method

The across-family linkage analysis was carried out using variance component (VC) based method (Sørensen et al., 2003). In LA with VC, the Identity by descent (IBD) probabilities between QTL alleles of any two founder haplotypes (Hs and Hm) are assumed to be zero, i.e. founder haplotypes were unrelated (Meuwissen et al. 2002). The sire haplotypes and the paternally inherited haplotypes of the sons are used to compute the probability of inheriting the paternal or maternal QTL allele from the sire (Freyer et al. 2004) and computed the IBD matrix using a recursive algorithm (Wang et al., 1995). The IBD matrices were computed at the midpoint of each marker bracket along the chromosome and used in the subsequent variance component estimation procedure. The fraction of the total additive genetic variance explained by the QTL was estimated as 2σ²ₕ/(2σ²ₕ+σ²ᵤ) where σ²ₕ and σ²ᵤ correspond respectively to the variance component associated with the haplotypes effect and the additive polygenic effect.

Variance component analysis. Single trait single QTL analysis.

Each trait was analysed separately using linkage analysis. The full model can be expressed as:

y=Xβ+Zu+Wq+e, (1)

where y is a vector of n EBVs, X is a known design matrix, β is a vector of unknown fixed effects, which is in this case only the mean, Z is a matrix relating to individuals, u is a vector of additive polygenic effects, W is a known matrix relating each individual record to its unknown additive QTL effect, q is a vector of unknown additive QTL effects of individuals and e is a vector of residuals. The random variables u, q and e are assumed to be multivariate normally distributed and mutually independent (Lund et al., 2003).

### Multi-Trait Multi-QTL Analysis

Multi-trait analysis was performed. Model (1) can be extended to a multi-trait multi-QTL model as described in Model (2) following Lund et al., 2003.

The traits are modeled using the following linear mixed model with n_{q} QTL:

$$y = μ + Za + {\sum }_{i = 1}^{{n}_{q}} {Wh}_{i} + e , \qquad ( 2 )$$

where y is a vector of observations for n sons recorded on t traits, μ is a vector of overall trait means, Z and W is known matrices associating the observations of each son to its polygenic and QTL effects, a is a vector of polygenic effects of sires and their sons, hᵢ is a vector of QTL haplotypes effects of sires and their sons for the i'th QTL and e is a vector of residuals. The random variables a, hᵢ and e are assumed to be multivariate normally distributed (MVN) and mutually uncorrelated. Specifically, a is MVN (0, G\textcircled{x}A), hᵢ is MVN (0, Kᵢ\textcircled{x}IBDᵢ) and e is MVN (0, E\textcircled{x}×I). Matrices G, K and E include variances and covariances among the traits due to polygenic effects, QTL effects and residuals effects. The symbol \textcircled{x} represents the Kronecker product. A is the additive relationship matrix that describe the covariance structure among the polygenic effects, IBDᵢ is the identity by descent (IBD) matrix that describes the covariance structure among the effects for the i'th QTL, and I is the identity matrix.

### Combined Linkage and Linkage Disequilibrium Analysis

In combined linkage and linkage disequilibrium analysis, the IBD probabilities between QTL alleles of any two founder haplotypes were computed using the method described by Meuwissen and Goddard (2001). This method approximates the probability that the two haplotypes are IBD at a putative QTL conditional on the identity-by-state (IBS) status of flanking markers, on the basis of coalescence theory (Hudson, 1985). Briefly, the IBD probability at the QTL is based on the similarity of the marker haplotypes surrounding alleles that surround the position: i.e. many (non) identical marker alleles near the position imply high (low) IBD probability at the map position. The actual level of IBD probabilities is affected by the effective population size, Ne. The probability of coalescence between the current and an arbitrary base generation, Tg generations ago is calculated given the marker alleles that both haplotypes have in common (Hudson, 1985). It is not easy to estimate Tg and Ne from the observed data. Simulation studies show that the estimate of QTL position is relatively insensitive to choice of Ne and Tg (Meuwissen and Goddard, 2000). Therefore we used the values of Tg=100 and Ne=100. Windows of 10 markers were considered to compute the IBD probabilities. We also used 4-markers window to compute IBD probabilities at the area of LDLA peak to examine if 4 markers were sufficient to reproduce the peak already identified by 10-marker haplotypes. Founder haplotypes were grouped into functionally distinct clusters. We used (1-IBDᵢⱼ) as a distance measure and applied the hierarchical clustering algorithm average linkage to generate a rooted dendrogram representing the genetic relationship between all founder haplotypes. The tree is scanned downward from the root and branches are cut until nodes are reached such that all coalescing haplotypes have a distance measure (1-IBDᵢⱼ)<Tc. A cluster is defined as a group of haplotypes that coalesce into a common node. Haplotypes within a cluster are assumed to carry identical QTL allele (IBD probability=1.0) whereas haplotypes from different clusters carry distinct QTL alleles and are therefore considered to be independent (IBD probability=0). Therefore the upper part of the IBD matrix corresponding to the linkage disequilibrium information is an identity matrix corresponding to the distinct founder haplotypes. The lower part of the IBD matrix corresponding to the linkage information in the paternal haplotypes of the sons is build using a recursive algorithm (Wang et al., 1995). The IBD matrices were computed at the midpoints of each marker interval and used in the subsequent variance component estimation procedure.

### Estimation of Parameters

The variance components were estimated using the average information restricted maximum likelihood algorithm (Jensen et al., 1997). The restricted likelihood was maximized with respect to the variance components associated with the random effects in the model. Maximizing a sequence of restricted likelihoods over a grid of specific positions yields a profile of the restricted likelihood of the QTL position (Sørensen et al., 2003). The parameters were estimated at the mid point of each marker bracket along the chromosome.

### Significance Level

Significance thresholds for the variance-component analyses were calculated using a quick method to compute approximate threshold levels that control the genome-wise type I error (Piepho, 2001). Hypothesis tests for the presence of QTL were based on the asymptotic distribution of the likelihood ratio test (LRT) statistic, LRT=−2 ln(L_{reduced}−L_{full}), where L_{reduced} and L_{full} were the maximized likelihoods under the reduced model and full model, respectively. The reduced model always excluded the QTL effect for the chromosome being analyzed. This method is an alternative to permutation procedures and is applicable in complex situations. It requires the LRT from each of the putative QTL positions along the chromosome, the number of chromosomes, the degrees of freedom (df) for the LRT (df=number of parameters of H_{full}−number of parameters of H_{reduced}), and the chromosome-wise type I error rate. A significance level of 5% chromosome wise was considered to be significant.

### Results BTA9

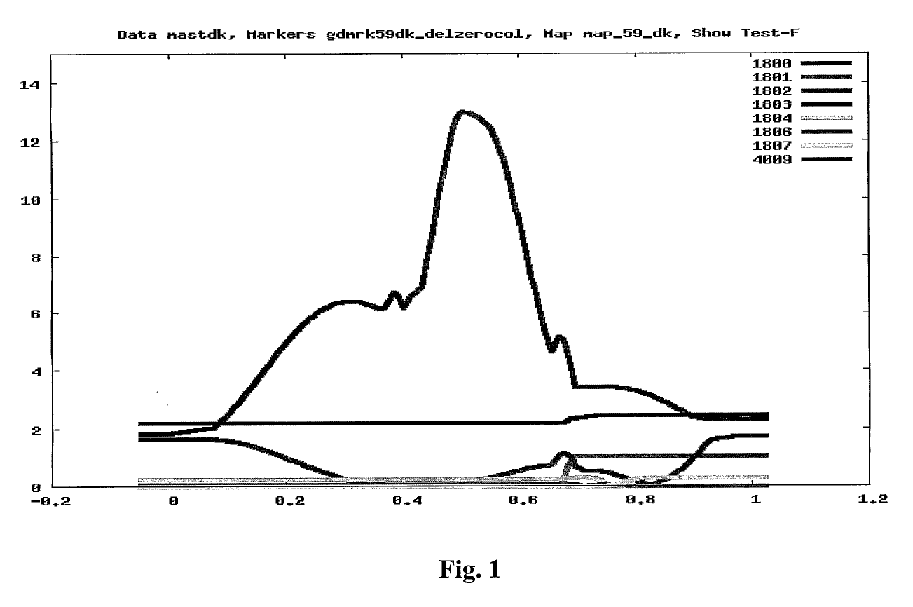

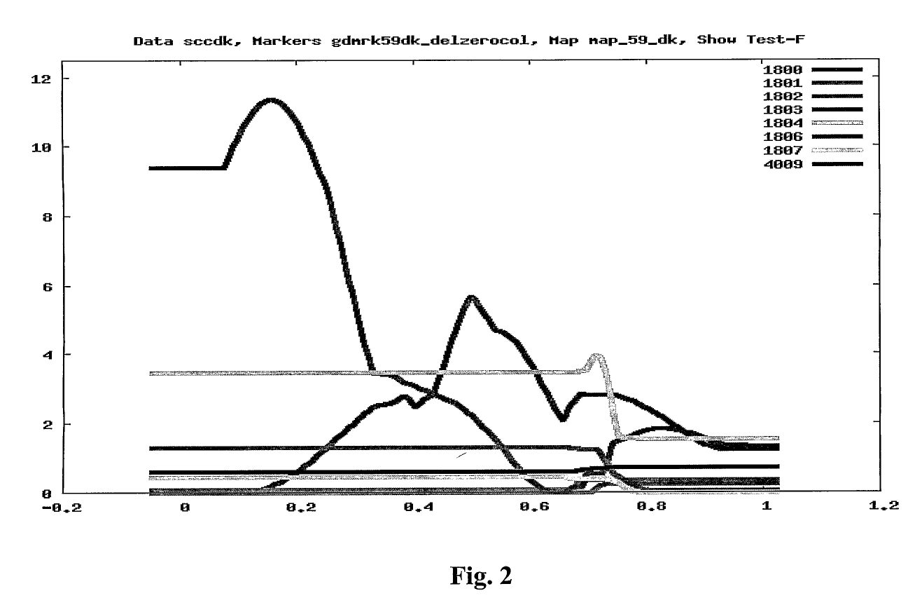

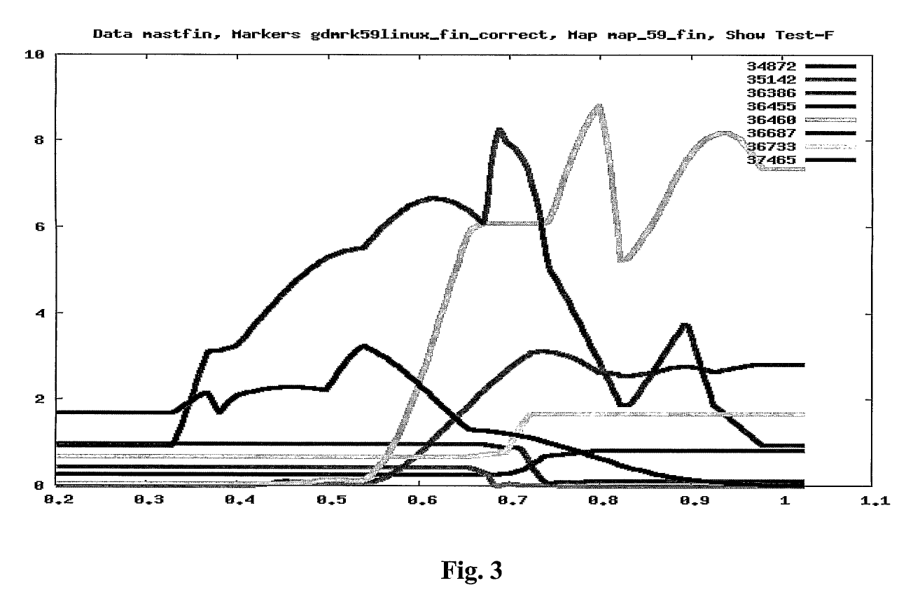

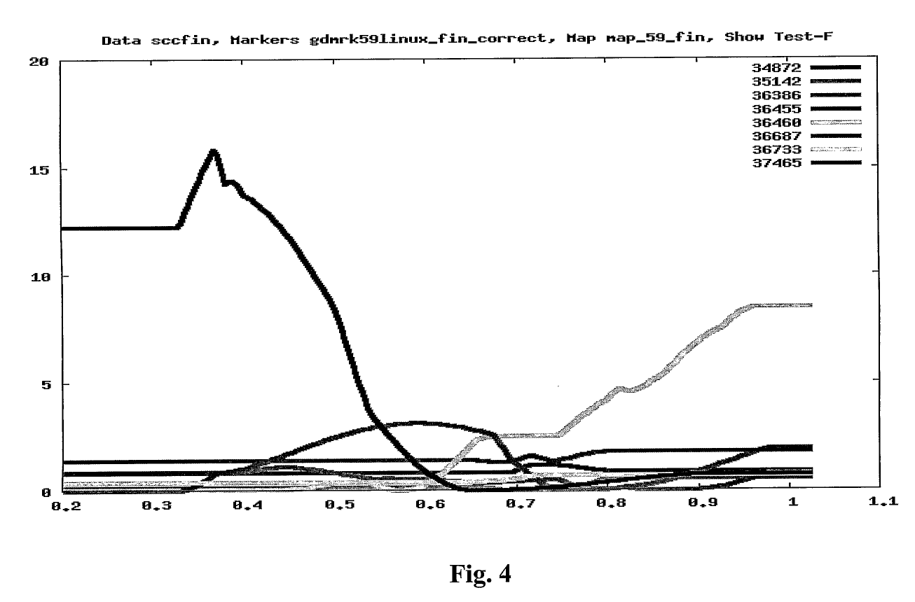

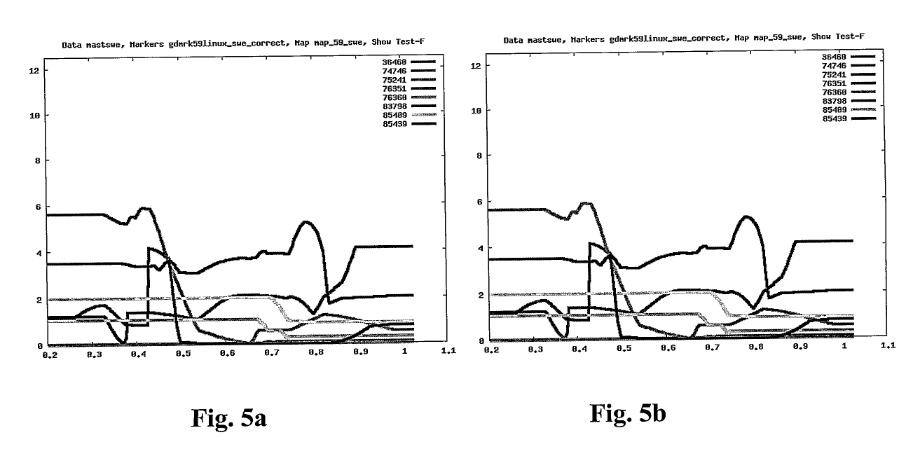

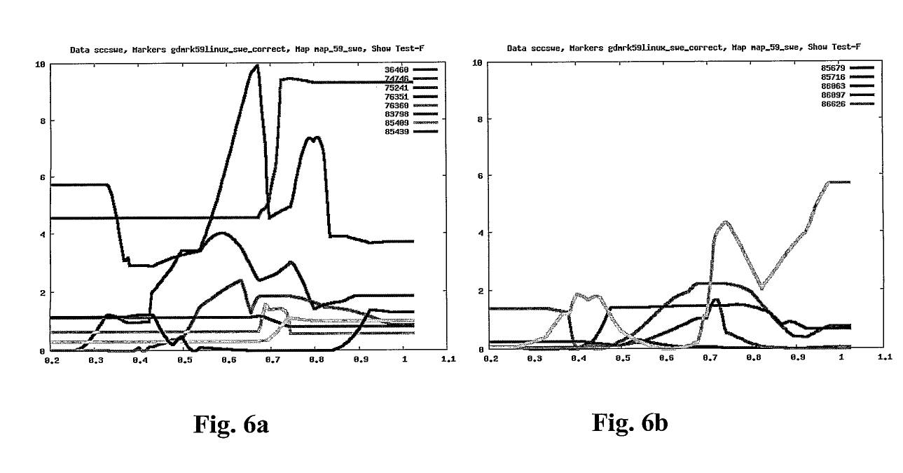

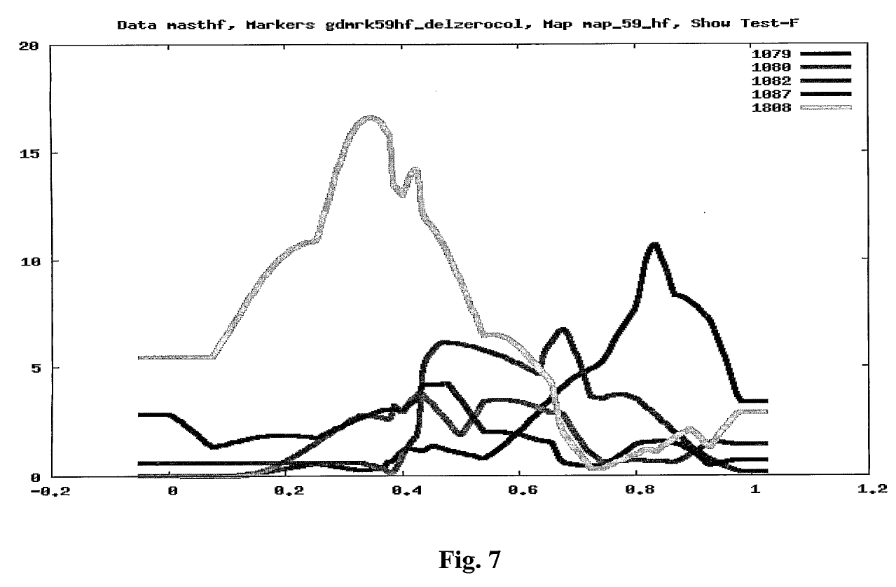

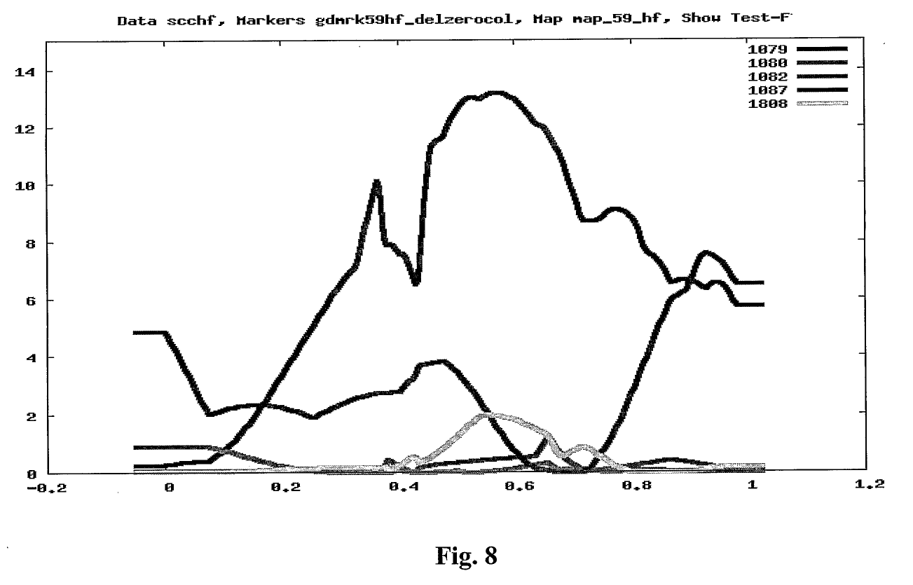

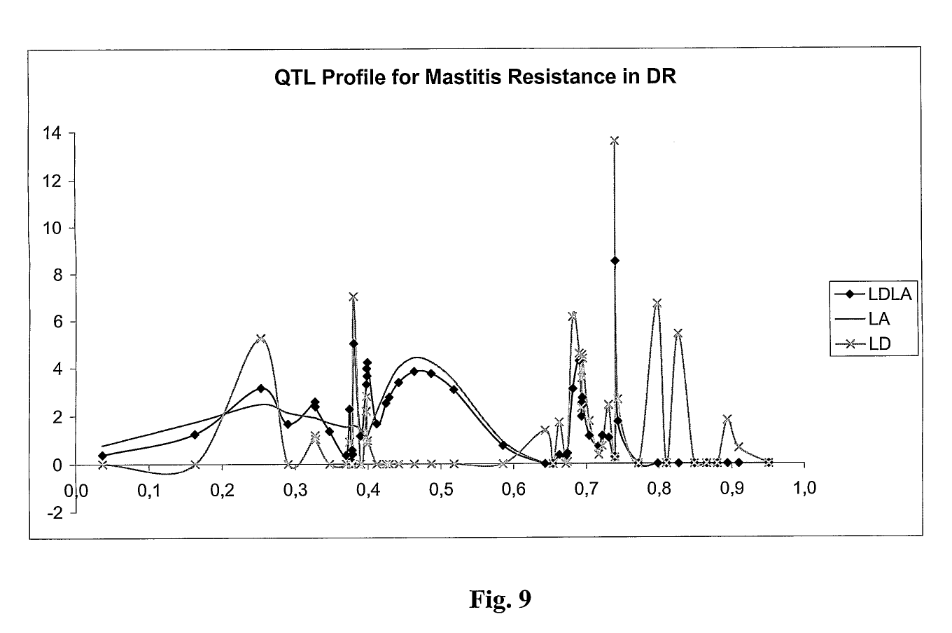

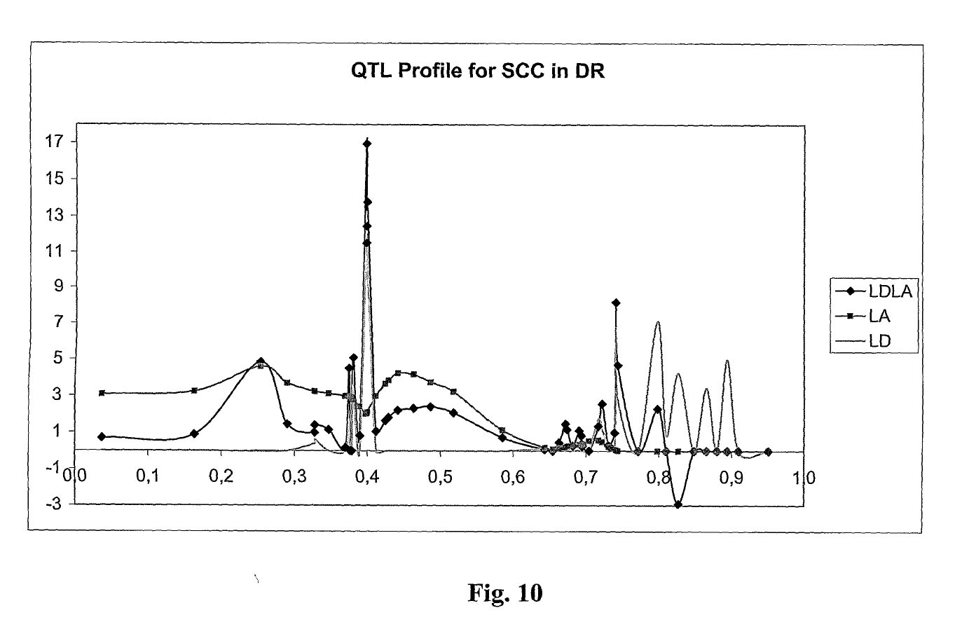

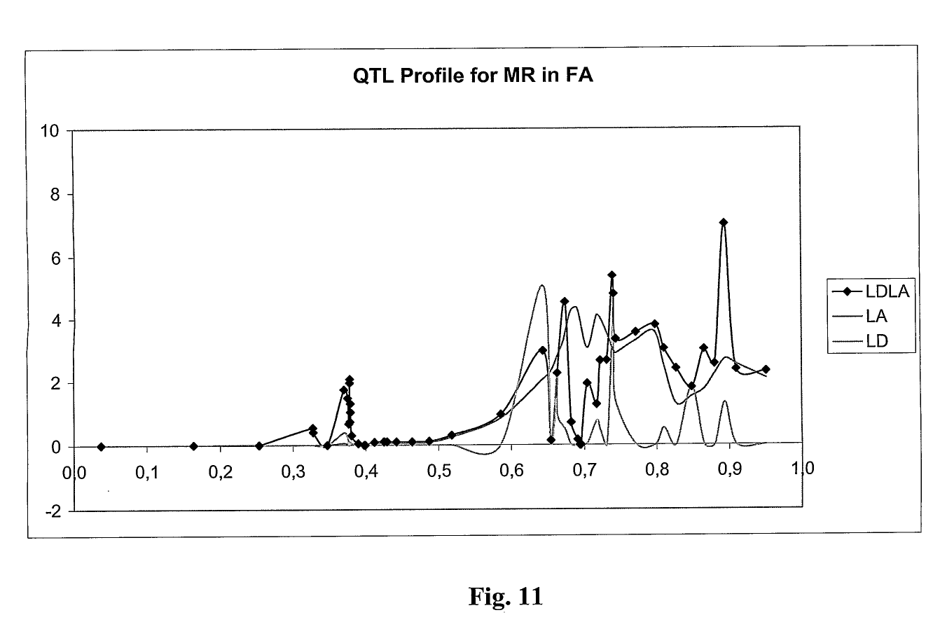

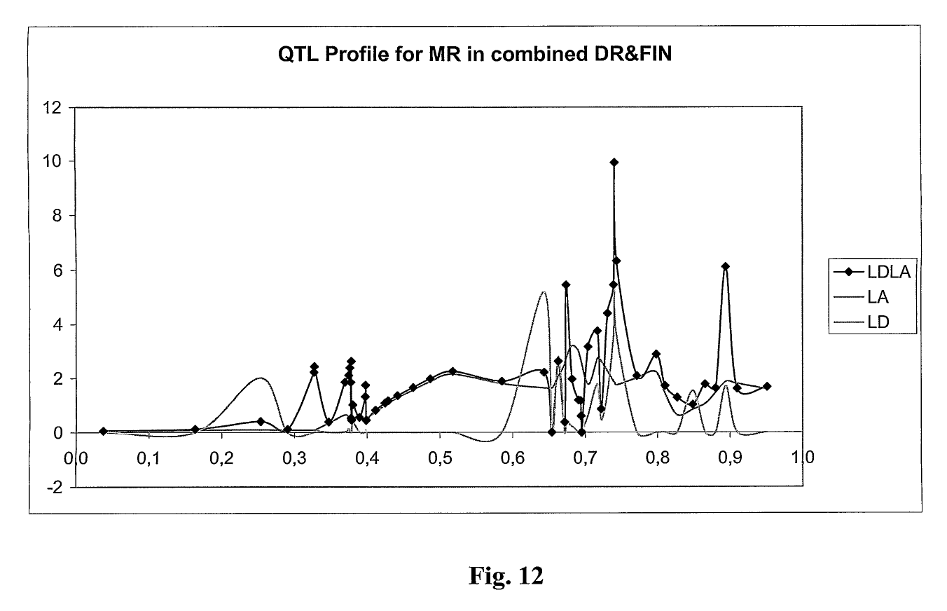

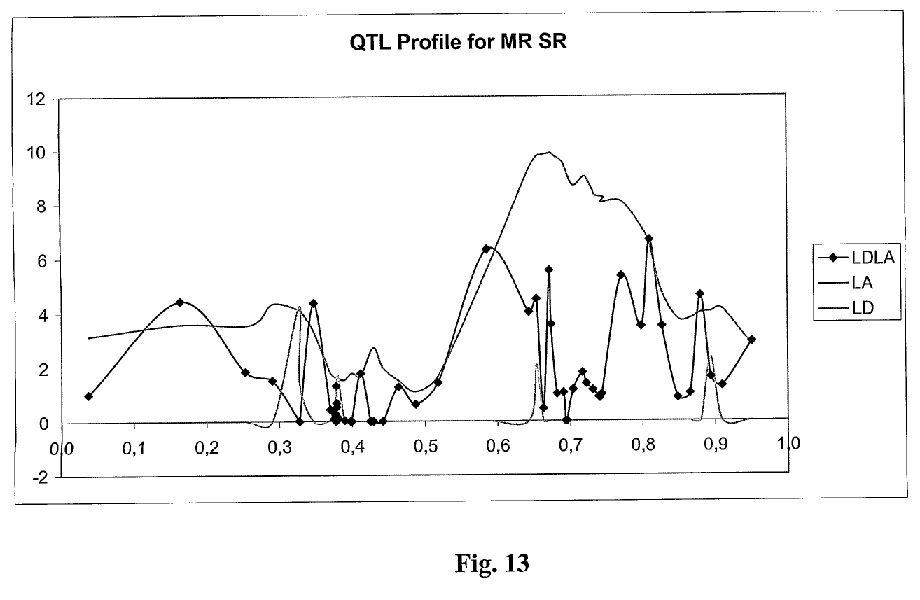

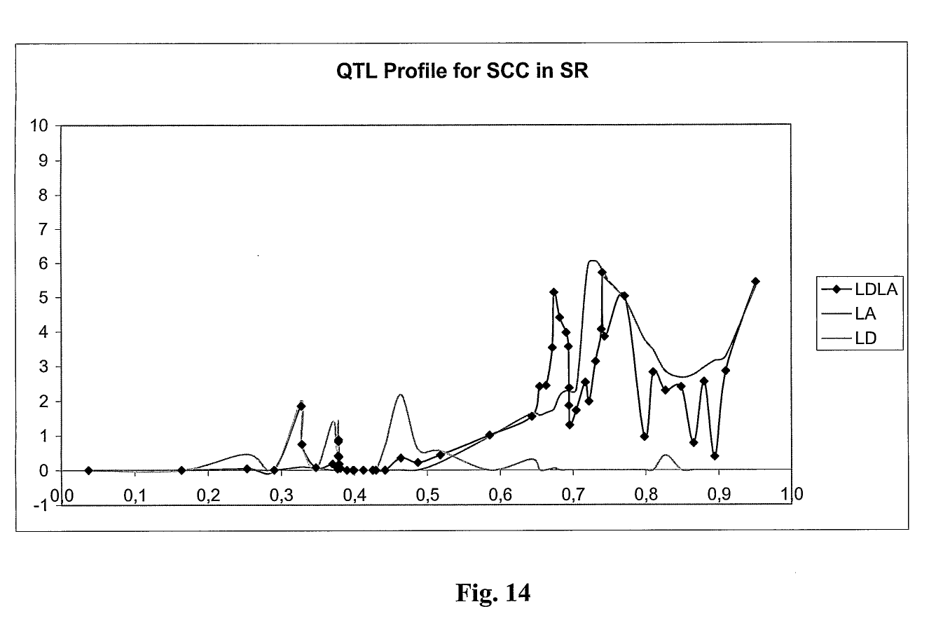

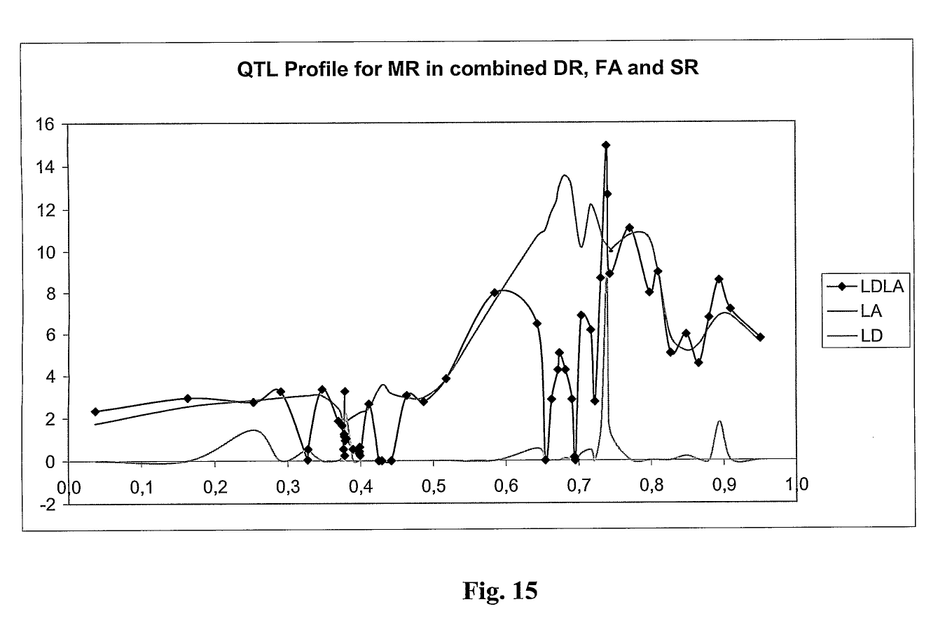

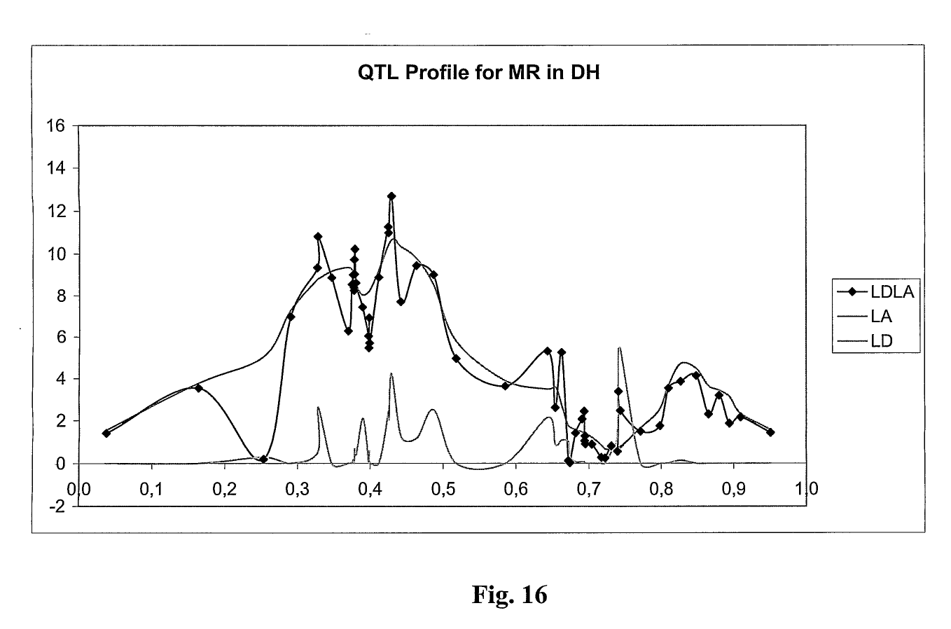

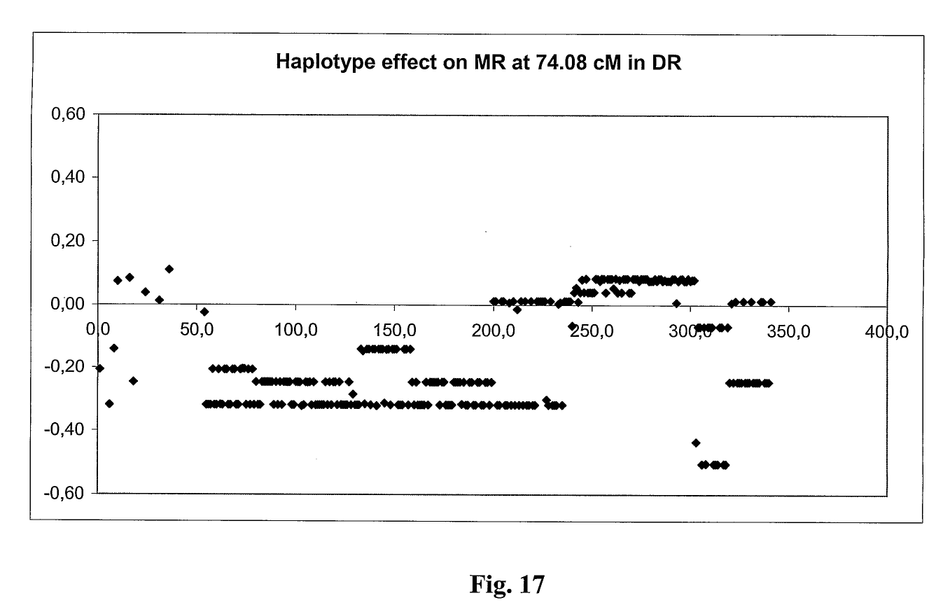

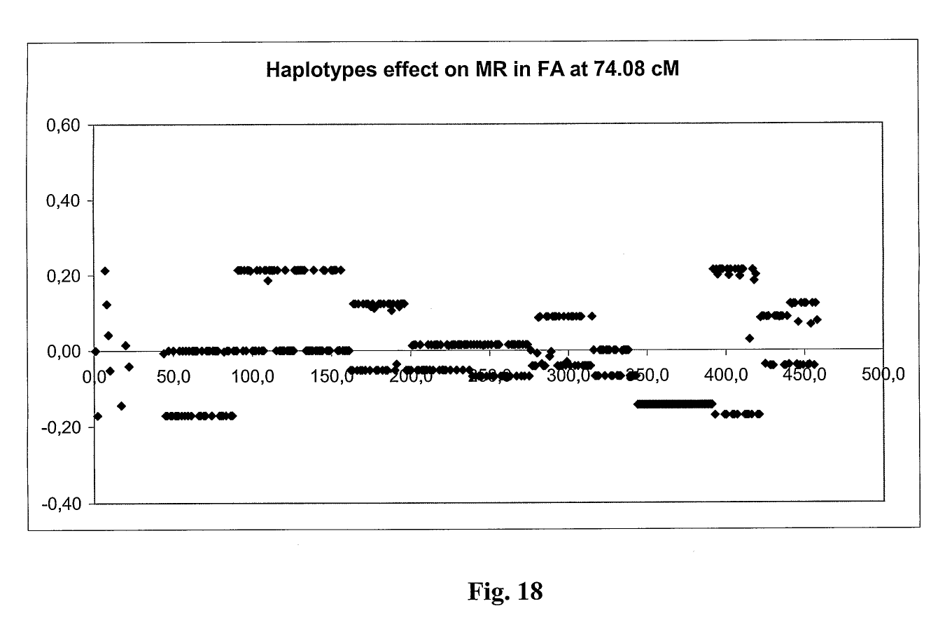

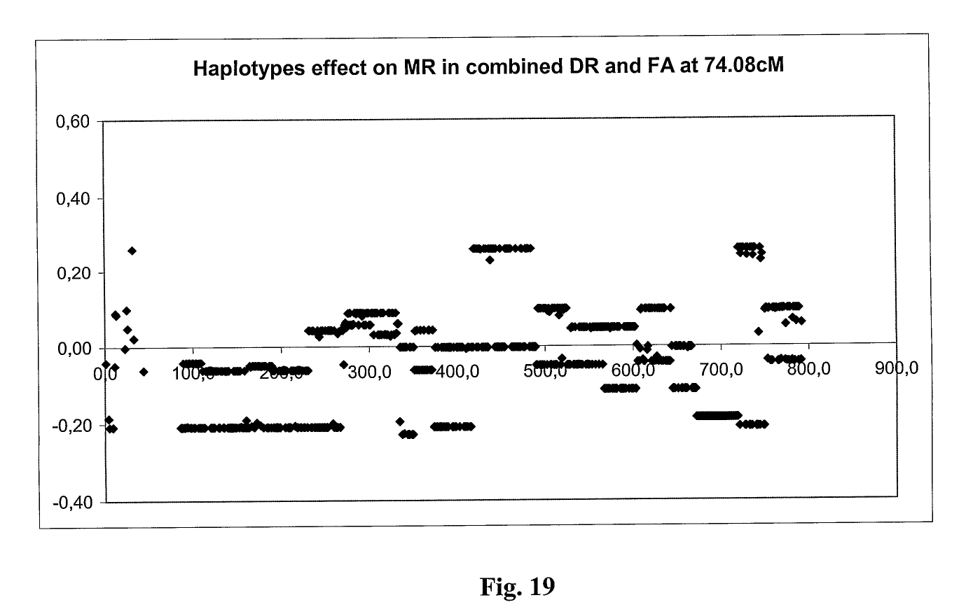

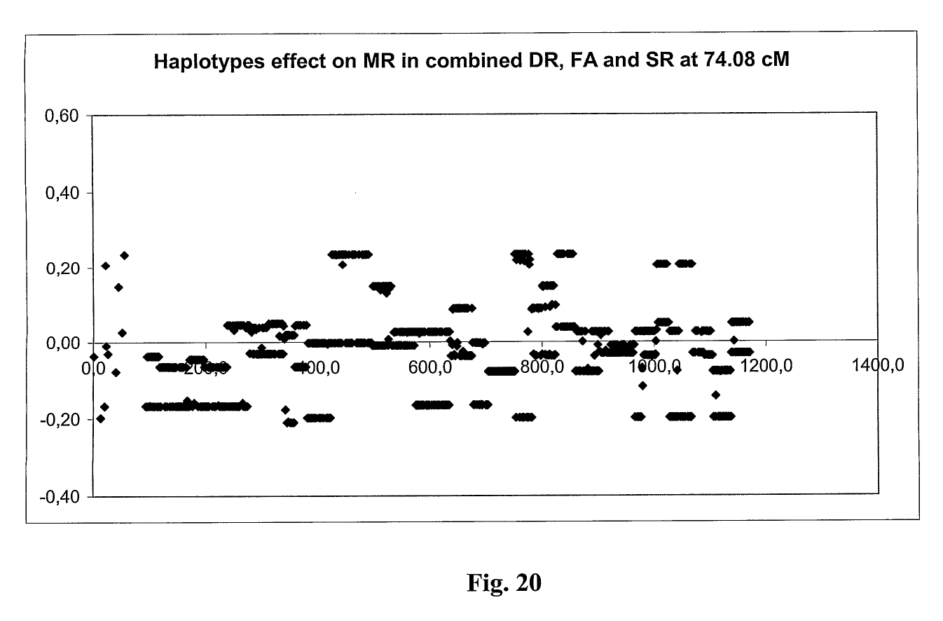

In table 53 the results from the regression analysis for BTA9 are presented. FIGS. 1 to 8 present the QTL graphs for the regression analysis. The variance component method was used to detect QTL across families QTL analysis. FIGS. 9 to 16 present the LD, LDLA and LD profile for the QTL in a variance component based method. FIGS. 17 to 20 present the haplotypes effects.

### Danish Red

Within family regression analysis revealed that QTL for CM and SCS are segregating in two families in DR breed. The QTL for two traits were not located in the same interval. In across-family linkage analysis using VC method, the QTL effects were not significant. With LDLA and LD analyses, high QTL peaks were observed at 74.08 cM between markers BMS2819 and INRA144. The peak LRT in LD analysis (13.6) was higher than the peak LRT (8.51) observed in combined LDLA analysis. This QTL explained 44% and 22% of the additive genetic variance and phenotypic variance for CM respectively. By default 10 marker haplotypes (five markers on each side of the putative position) were used to estimate the IBD probability of a location. We also used 4 marker haplotypes i.e. 2 markers on each side of the putative position, and observed similar LDLA/LD peak within these four marker (BM4208-BMS2819-INRA144-INRA084) haplotypes. A LDLA combined peak for SCS was also observed within these 4 markers bracket in this breed. The dam haplotypes with IBD probability of 0.90 or above were clustered together. There were 305 founder haplotypes in DR before clustering which reduced to 54 clusters after clustering. Five clusters had frequency higher than 5% and the largest cluster had a frequency of 10% and five sire haplotypes were also clustered with the largest cluster. The haplotypes effects for these 54 haplotypes and also the haplotypes received from the sires were estimated. The haplotypes associated with high and low mastitis resistance were identified.

### Finnish Ayrshire

The QTL affecting CM and SCS were found segregating when within family regression analysis was performed in Finish Ayrshire families. The QTL for CM was located in the interval of 58 to 79 cM. The QTL affecting SCS was located in between 32 to 44 cM with the peak LRT statistics at 37 cM. The combined LDLA peak for CM QTL over LA profile was observed within the markers BM4208-BMS2819-INRA144. The LD peak was also observed in the same region for CM. One LDLA peak for SCs over LA profile was observed at 38 cM between the markers DIK2810 and DIK5364. Four percent of the total variance in CM was explained by the QTL at 74 cM and this QTL showed no effect on SCS in FA. The QTL at 38 cM explained 18% of the total variance in SCS and it had very small effect on CM. At the highest LDLA peak in CM i.e. in the mid interval between markers BM4208 and BMS2819, 442 founder haplotypes grouped into 38 clusters when the clustering probability of 0.90 was applied. There were nine clusters with frequency higher than 5%. The biggest cluster had a frequency of 14%. The haplotypes associated with high and low mastitis resistance were identified.

### Swedish Red and White

Similar to DR and FA cattle, QTL affecting CM and SCS were also observed segregating on BTA9 in Swedish Red and White cattle when within-family regression analyses were performed. Both the QTL for CM and SCS were located in the same interval. The CM QTL was significant (P<0.01) in across-family LA analysis. The SCS QTL was not significant in across-family LA analysis. The peak LRT for SCS was at 73cM. The peak test statistics for CM QTL in across-family analysis was at 67.4 cM with the QTL interval was between 59 and 81 cM. Though the LRT statistics was highly significant in across-family LA analysis, no LDLA peak over LA profile was observed for this QTL in SRB cattle. At the peak LRT statistics location in LA analysis, the QTL variance was 25% of the total variance of CM trait. LDLA peaks for CM QTL of DR and FA breeds fall within the LA profile observed in SRB. Though no LDLA peak was observed in SRB data for CM, there were lot of clustering in SRB in the marker intervals where the peaks in DR and FA was located. For example at the mid interval between BM4208-BMS2819, where the highest LDLA peak is located in FA and in the neighbouring interval the DR peak (BMS2819-INRA144), there were 37 and 48 clusters respectively, out of 400 total founder haplotypes in SRB.

### Danish Holstein

QTL affecting CM and SCS was also segregating in Danish Holstein cattle revealed in within-family regression analysis. The CM QTL was significant (P<0.01) in across-family LA analysis with VC method. Though small LDLA peaks over LA profile was observed, but no convincing LD peak was seen for the QTL in DH. The highest LRT statistics for CM QTL in LA was at 42.9 cM with the LRT statistics of 10.6 and the QTL interval was quite large spreading from 29 to 51 cM. One small LD peak for CM QTL coincides with the LD peaks observed in DR and FA population at 74 cM. The SCC QTL has peak test statistics at 48.7 cM with an interval from 44 to 58 cM. The part of total variance explained by the QTL taking the highest peaks in respective LA were 27 and 17% for CM and SCS respectively. The highest LD peak for CM was at 73.35cM, the region where high LD peak for DR was observed. No LD peak for SCS was observed in DH.

### Across Breed Analysis

Within-breed LA, LDLA and LD analyses revealed that the QTL affecting CM were segregating at around 74 cM in more than one population. Therefore, across-breed QTL analyses were carried out combing data across different breeds in the study. The results of across-breed QTL analysis are presented in Tables 20, 21 and 22. The LDLA peak for CM QTL in DR and FA cattle was located in the neighbouring marker intervals when within breed analyses were done. However, a high LDLA peak of CM was observed in the marker bracket (BMS2819-INRA144) when combined data of DR and FA were analyzed and also coincides with the LD peak. The combined analysis of FA and SRB data didn't gave any higher LDLA peak over LA profile, however, the LD peak was observed at the same marker interval at 74 cM. The analyses of combined DR, FA and SRB data also gave the higher LDLA peak over LA in the same region i.e. BM4208-BMS2819-INRA144. The LD peak was also at the same location which authenticated the higher LDLA peak over LA. The joint analysis of DR and FA showed a high LDLA peak at 38 cM between the markers DIK2810 and DIK5364 for SCS QTL. LDLA peak at the same location was also observed for SCS QTL in combined analysis of FA and SRB. However, this LDLA peak disappeared when DR, FA and SRB were analyzed together.

### Multi-Trait Analysis

SCS is an indicator trait of mastitis resistance. It was expected that many of genes responsible of CM will also have effect on SCS. Therefore, multi-trait analysis of CM and SCS was carried out to test if the QTL segregating on BTA9 have pleiotropic effect on both the traits or they are linked QTL. Though the single-trait LDLA analysis of DR data showed the LDLA peak of CM and SCS at the same marker interval i.e. between BMS2819 and INRA144, the combined analysis of 2-traits gave LDLA peak at 69.1 cM in between the markers SLU2 and C6orf93. In within-breed analysis FA, SR did not show LDLA peak for the model with QTL affecting both CM and SCS. However when the three breeds, DR, FA and SRB were combined and analyzed with a 2-trait model, LDLA peak with LRT statistics of 19.7 was observed in the marker interval INRA144 and INRA084.

### Haplotype Analysis

QTL fine mapping results mentioned above, points towards a QTL segregating for CM within the 4-marker region, BM4208-BMS2819-INRA144-INRA084. Therefore the clustering of founder haplotypes and haplotypes effects were studied at the midpoint between the markers BMS2819 and INRA144. This was done within breeds and also across three breeds DR, FA and SRB as these three breeds are related in their origin. The haplotypes associated with high and low mastitis resistance were identified.

TABLE 53
With-family linkage analysis using regression interval analysis.

| Breed | Mastitis resistance (CM) | | | Somatic Cell Count | | |
|---|---|---|---|---|---|---|
| and Sire | Position | F- | P- | Position | F- | P- |
| No. | (Morgan) | values | values | (Morgan) | values | values |
| Danish | | | | | | |
| Holstein | | | | | | |
| 1079 | 0.829 | 10.73 | 0.99 | 0.596 | 13.19 | 1.00 |
| 1080 | 0.432 | 3.79 | 0.73 | 0.072 | 0.91 | 0.12 |
| 1082 | 0.643 | 6.76 | 0.92 | 0.654 | 1.18 | 0.21 |
| 1087 | 0.474 | 4.22 | 0.76 | 0.919 | 7.58 | 0.95 |
| 1808 | 0.347 | 16.61 | 1.00 | 0.596 | 1.97 | 0.33 |
| Across- | 0.432 | 4.90 | 1.00 | 0.523 | 3.43 | 0.97 |
| family | | | | | | |
| Danish | | | | | | |
| Red | | | | | | |
| 1800 | 0.501 | 13.00 | 0.99 | 0.501 | 5.65 | 0.86 |
| 1801 | 0.728 | 1.06 | 0.57 | 0.728 | 0.36 | 0.28 |
| 1802 | 0.699 | 0.07 | 0.07 | 0.961 | 0.23 | 0.21 |
| 1803 | 1.009 | 1.75 | 0.37 | 0.156 | 11.35 | 0.99 |
| 1804 | 0.718 | 0.32 | 0.18 | 0.712 | 3.96 | 0.88 |
| 1806 | 0.739 | 0.07 | 0.05 | 0.654 | 1.30 | 0.60 |
| 1807 | 0.363 | 0.28 | 0.24 | 0.442 | 0.45 | 0.34 |
| 4009 | 0.696 | 2.46 | 0.86 | 0.358 | 0.73 | 0.59 |
| Across- | 0.502 | 1.95 | 0.81 | 0.497 | 1.77 | 0.71 |
| family | | | | | | |
| Finnish | | | | | | |
| Ayrshire | | | | | | |
| 34872 | 0.913 | 0.83 | 0.51 | 0.416 | 1.17 | 0.62 |
| 35142 | 0.363 | 0.44 | 0.26 | 0.358 | 0.55 | 0.32 |
| 36386 | 1.003 | 3.14 | 0.71 | 1.003 | 1.95 | 0.50 |
| 36455 | 0.617 | 0.96 | 0.49 | 0.903 | 1.76 | 0.69 |
| 36460 | 0.792 | 8.81 | 0.97 | 0.945 | 8.49 | 0.97 |
| 36687 | 0.718 | 8.27 | 0.95 | 0.564 | 3.11 | 0.63 |
| 36733 | 0.728 | 1.67 | 0.74 | 0.728 | 0.66 | 0.49 |
| 37465 | 0.538 | 3.24 | 0.71 | 0.368 | 15.85 | 1.00 |
| 37505 | 0.358 | 1.31 | 0.58 | 0.358 | 1.66 | 0.66 |
| 38393 | 0.358 | 0.64 | 0.55 | 0.384 | 1.99 | 0.80 |
| 38651 | 0.808 | 1.33 | 0.56 | 0.998 | 2.17 | 0.72 |
| Across- | 0.687 | 2.03 | 0.90 | 0.978 | 1.94 | 0.89 |
| family | | | | | | |
| Swedish | | | | | | |
| Red | | | | | | |
| 36460 | 0.792 | 5.22 | 0.85 | 0.945 | 9.92 | 0.97 |
| 74746 | 0.426 | 5.89 | 0.91 | 0.638 | 2.38 | 0.58 |
| 75241 | 0.702 | 0.10 | 0.06 | 0.744 | 9.47 | 0.99 |
| 76351 | 0.913 | 4.13 | 0.78 | 0.336 | 5.74 | 0.88 |
| 76360 | 0.654 | 1.07 | 0.48 | 0.686 | 1.57 | 0.61 |
| 83798 | 0.834 | 0.09 | 0.09 | 0.670 | 1.17 | 0.59 |
| 85409 | 0.686 | 2.00 | 0.73 | 0.739 | 1.10 | 0.55 |
| 85439 | 0.363 | 3.62 | 0.74 | 0.919 | 1.38 | 0.33 |
| 85679 | 0.903 | 10.82 | 0.97 | 0.336 | 0.32 | 0.01 |
| 85716 | 0.739 | 11.77 | 0.98 | 0.649 | 2.21 | 0.49 |
| 86063 | 0.723 | 4.18 | 0.79 | 0.718 | 1.67 | 0.42 |
| 86097 | 0.432 | 1.68 | 0.53 | 0.760 | 1.51 | 0.50 |
| 86626 | 0.416 | 5.19 | 0.84 | 1.009 | 5.72 | 0.87 |
| Across- | 0.674 | 3.18 | 1.00 | 0.724 | 2.22 | 0.94 |
| family | | | | | | |

TABLE 54
Summary of across-family linkage analysis (LA) using
variance component method

| | | Position | Peak LRT | |
|---|---|---|---|---|
| Breed | Trait | (Morgan) | statistics | Marker interval |
| Danish Red (DR) | CM | 0.464 | 4.43 | BM4208-DIK4926 |
| | SCS | 0.253 | 4.58 | BMS2504-DIK2892 |
| Finish Ayrshire | CM | 0.682 | 4.19 | BM7209-SLU2 |
| (FA) | SCS | 0.370 | 5.12 | BMS817-BMS555 |
| Swedish Red | CM | 0.674 | 9.89 | DIK2145-BM7209 |
| (SR) | SCS | 0.731 | 6.05 | BM7234-BM4208 |
| Danish Holstein | CM | 0.429 | 10.63 | DIK2303-DIK4720 |
| (DH) | SCS | 0.487 | 6.72 | DIK4926-BMS1909 |
| DR + FA | CM | 0.682 | 3.20 | BM7209-SLU2 |
| | SCS | 0.370 | 6.57 | BMS817-BMS555 |
| FA + SR | CM | 0.682 | 15.30 | BM7209-SLU2 |
| | SCS | 0.951 | 9.91 | BMS1943-BMS1967 |
| DR + FA + SR | CM | 0.682 | 13.53 | BM7209-SLU2 |
| | SCS | 0.951 | 8.51 | BMS1943-BMS1967 |

TABLE 55
Summary of linkage disequilibrium and linkage analysis
(LDLA) using variance component method

| | | Position | Peak LRT | |
|---|---|---|---|---|
| Breed | Trait | (Morgan) | statistics | Marker interval |
| Danish Red (DR) | CM | 0.741 | 8.51 | BM2819-INRA144 |
| | SCS | 0.398 | 16.95 | DIK2741-TGLA261 |
| Finish Ayrshire | CM | 0.739 | 5.38 | BM4208-BMS2819 |
| (FA) | SCS | 0.381 | 7.26 | DIK2810-DIK5364 |
| Swedish Red (SR) | CM | 0.672 | 5.56 | BMS1724-DIK2145 |
| | SCS | 0.741 | 5.71 | BMS2819-INRA144 |
| Danish Holstein | CM | 0.429 | 12.69 | DIK2303-DIK4720 |
| (DH) | SCS | 0.464 | 7.85 | BM4204-DIK4926 |
| DR + FA | CM | 0.741 | 9.93 | BMS2819-INRA144 |
| | SCS | 0.381 | 9.61 | DIK2810-DIK5364 |
| FA + SR | CM | 0.739 | 10.98 | BM4208-BMS2819 |
| | SCS | 0.691 | 6.26 | SLU2-C6orf93 |
| DR + FA + SR | CM | 0.739 | 14.90 | BM4208-BMS2819 |
| | SCS | 0.741 | 6.69 | BMS2819-INRA144 |

TABLE 56
Summary of across-family linkage disequilibrium (LD)
analysis using variance component method

| | | Position | Peak LRT | |
|---|---|---|---|---|
| Breed | Trait | (Morgan) | statistics | Marker interval |
| Danish Red (DR) | CM | 0.741 | 13.60 | BM2819-INRA144 |
| | SCS | 0.398 | 12.95 | DIK2741-TGLA261 |
| Finish Ayrshire | CM | 0.739 | 3.64 | BM4208-BMS2819 |
| (FA) | SCS | — | <1.0 | — |
| Swedish Red | CM | 0.327 | 4.26 | DIK3002-DIK3003 |
| | SCS | 0.464 | 2.18 | BM4204-DIK4926 |
| Danish Holstein | CM | 0.744 | 5.45 | INRA144-INRA084 |
| (DH) | SCS | 0.731 | 1.49 | BM7234-BM4208 |
| DR + FA | CM | 0.741 | 5.49 | BMS2819-INRA144 |
| | SCS | 0.398 | 1.99 | TGLA261-ILSTS013 |
| FA + SR | CM | 0.739 | 5.39 | BM4208-BMS2819 |
| | SCS | 0.370 | 1.73 | BMS817-BMS555 |
| DR + FA + SR | CM | 0.739 | 8.94 | BM4208-BMS2819 |
| | SCS | 0.253 | 4.08 | BMS2504-DIK2892 |

### Example 2

### BTA11

### Statistical Analysis

A number of statistical methods as described below were used in the determination of genetic markers associated or linked to mastitis and thus mastitis resistance.

### QTL Analysis

Linkage analysis (LA) is used to identify QTL by typing genetic markers in families to chromosome regions that are associated with disease or trait values within pedigrees more often than are expected by chance. Such linked regions are more likely to contain a casual genetic variant. The data was analysed with a series of models. Three complementary approaches were used: (i) within half-sib family segregation analysis by regression based method (Haley and Knott, 1992) using GDQTL software (B. Guldbrandsten, 2005 personal communication); (ii) across family linkage analysis using variance component method, and (iii) combined linkage disequilibrium linkage analysis (LDLA) using variance component method. Each family was individually analyzed by using GDQTL to determine the sire's QTL segregation status for each trait. Permutation test (n=10,000) was used to determine chromosome wise significance level for each sire (Churchill and Doerge, 1994). The next step was across family linkage analyses using variance component based method (Sørensen et al., 2003) combining the data set from families segregating for QTL, regardless of the trait and QTL position. Thresholds were calculated using the method presented by Piepho (2001). The third step was combined LDLA analyses (Lund et al. 2003) including all the segregating and non-segregating families. Multi-trait and multi-QTL models were analyzed to separate pleiotropic QTL from linked QTL. When the QTL was observed segregating in the same region of the BTA11 in more than one breed, the LDLA analyses were performed combing the data across breeds.

### Variance Component Method

The across-family linkage analysis was carried out using variance component (VC) based method (Sørensen et al., 2003). In LA with VC, the Identity by descent (IBD) probabilities between QTL alleles of any two founder haplotypes (Hs and Hm) are assumed to be zero, i.e. founder haplotypes were unrelated (Meuwissen et al. 2002). The sire haplotypes and the paternally inherited haplotypes of the sons are used to compute the probability of inheriting the paternal or maternal QTL allele from the sire (Freyer et al. 2004) and computed the IBD matrix using a recursive algorithm (Wang et al., 1995). The IBD matrices were computed at the midpoint of each marker bracket along the chromosome and used in the subsequent variance component estimation procedure. The fraction of the total additive genetic variance explained by the QTL was estimated as 2σ²ₕ/(2σ²ₕ+σ²ᵤ) where σ²ₕ and σ²ᵤ correspond respectively to the variance component associated with the haplotypes effect and the additive polygenic effect.

Variance component analysis, Single trait single QTL analysis.

Each trait was analysed separately using linkage analysis. The full model can be expressed as:

y=Xβ+Zu+Wq+e, (1)

where y is a vector of n EBVs, X is a known design matrix, β is a vector of unknown fixed effects, which is in this case only the mean, Z is a matrix relating to individuals, u is a vector of additive polygenic effects, W is a known matrix relating each individual record to its unknown additive QTL effect, q is a vector of unknown additive QTL effects of individuals and e is a vector of residuals. The random variables u, q and e are assumed to be multivariate normally distributed and mutually independent (Lund et al., 2003).

### Multi-Trait Multi-QTL Analysis

Multi-trait analysis was performed. Model (1) can be extended to a multi-trait multi-QTL model as described in Model (2) following Lund et al., 2003.

The traits are modeled using the following linear mixed model with n_{q} QTL:

$$y = μ + Za + {\sum }_{i = 1}^{{n}_{q}} {Wh}_{i} + e , \qquad ( 2 )$$

where y is a vector of observations for n sons recorded on t traits, μ is a vector of overall trait means, Z and W is known matrices associating the observations of each son to its polygenic and QTL effects, a is a vector of polygenic effects of sires and their sons, hᵢ is a vector of QTL haplotypes effects of sires and their sons for the i'th QTL and e is a vector of residuals. The random variables a, hᵢ and e are assumed to be multivariate normally distributed (MVN) and mutually uncorrelated. Specifically, a is MVN (0, G\textcircled{x}A), hᵢ is MVN (0, Kᵢ\textcircled{x}IBDᵢ) and e is MVN (0, E\textcircled{x}I). Matrices G, K and E include variances and covariances among the traits due to polygenic effects, QTL effects and residuals effects. The symbol \textcircled{x}represents the Kronecker product. A is the additive relationship matrix that describe the covariance structure among the polygenic effects, IBDᵢ is the identity by descent (IBD) matrix that describes the covariance structure among the effects for the i'th QTL, and I is the identity matrix.

### Regression Analysis

Population allele frequencies at the markers were estimated using an EM-algorithm. Allele frequencies were subsequently assumed known without error. Phase in the sires was determined based on offspring marker types. Subsequently this phase was assumed known without error. Segregation probabilities at each map position were calculated using information from all markers on the chromosome simultaneously using Haldane's mapping function (Haldane, 1919). Phenotypes were regressed onto the segregation probabilities. Significance thresholds were calculated using permutation tests (Churchil and Doerge, 1994).

### Estimation of Parameters

The variance components were estimated using the average information restricted maximum likelihood algorithm (Jensen et al., 1997). The restricted likelihood was maximized with respect to the variance components associated with the random effects in the model. Maximizing a sequence of restricted likelihoods over a grid of specific positions yields a profile of the restricted likelihood of the QTL position (Sørensen et al., 2003). The parameters were estimated at the mid point of each marker bracket along the chromosome. The fraction of the total additive genetic variance explained by the QTL was estimated as 2σ²ₕ/(2σ²ₕ+σ²ᵤ) where σ²ₕ and σ²ᵤ correspond respectively to the variance component associated with the haplotypes effect and the additive polygenic effect.

### Estimation of IBD Probabilities

Linkage analysis: The IBD probabilities between QTL alleles of any two founder haplotypes (Hs and Hm) are assumed to be zero, i.e. founder haplotypes were unrelated (Meuwissen et al. 2002). The sire haplotypes and the paternally inherited haplotypes of the sons are used to compute the probability of inheriting the paternal or maternal QTL allele from the sire and the IBD matrix was computed using a recursive algorithm (Wang et al., 1995). The IBD matrices were computed at every 2 cM interval along the chromosome and used in the subsequent variance component estimation procedure.

### Combined Linkage and Linkage Disequilibrium Analysis

In combined linkage and linkage disequilibrium analysis, the IBD probabilities between QTL alleles of any two founder haplotypes were computed using the method described by Meuwissen and Goddard (2001). This method approximates the probability that the two haplotypes are IBD at a putative QTL conditional on the identity-by-state (IBS) status of flanking markers, on the basis of coalescence theory (Hudson, 1985). Briefly, the IBD probability at the QTL is based on the similarity of the marker haplotypes surrounding alleles that surround the position: i.e. many (non) identical marker alleles near the position imply high (low) IBD probability at the map position. The actual level of IBD probabilities is affected by the effective population size, Ne. The probability of coalescence between the current and an arbitrary base generation, Tg generations ago is calculated given the marker alleles that both haplotypes have in common (Hudson, 1985). It is not easy to estimate Tg and Ne from the observed data. Simulation studies show that the estimate of QTL position is relatively insensitive to choice of Ne and Tg (Meuwissen and Goddard, 2000). Therefore we used the values of Tg=100 and Ne=100. Windows of 10 markers were considered to compute the IBD probabilities. We also used different marker-window e.g. 6-marker, 4-markers etc. to compute IBD probabilities at the area of LDLA peak to examine if fewer markers were sufficient to explain the QTL variance detected by 10-marker haplotypes. Founder haplotypes were grouped into distinct clusters. We used (1-IBDᵢⱼ) as a distance measure and applied the hierarchical clustering algorithm average linkage to generate a rooted dendrogram representing the genetic relationship between all founder haplotypes. The tree is scanned downward from the root and branches are cut until nodes are reached such that all coalescing haplotypes have a distance measure (1-IBDᵢⱼ)<Tc. A cluster is defined as a group of haplotypes that coalesce into a common node. Haplotypes within a cluster are assumed to carry identical QTL allele (IBD probability=1.0) whereas haplotypes from different clusters carry distinct QTL alleles and are therefore considered to be independent (IBD probability=0). Therefore the upper part of the IBD matrix corresponding to the linkage disequilibrium information is an identity matrix corresponding to the distinct founder haplotypes. The lower part of the IBD matrix corresponding to the linkage information in the paternal haplotypes of the sons is build using a recursive algorithm (Wang et al., 1995). The IBD matrices were computed at the midpoints of each marker interval and used in the subsequent variance component estimation procedure.

### Significance Level

Significance thresholds for the variance-component analyses were calculated using a quick method to compute approximate threshold levels that control the genome-wise type I error (Piepho, 2001). Hypothesis tests for the presence of QTL were based on the asymptotic distribution of the likelihood ratio test (LRT) statistic, LRT=−2 ln(L_{reduced}−L_{full}), where L_{reduced} and L_{full} were the maximized likelihoods under the reduced model and full model, respectively. The reduced model always excluded the QTL effect for the chromosome being analyzed. This method is an alternative to permutation procedures and is applicable in complex situations. It requires the LRT from each of the putative QTL positions along the chromosome, the number of chromosomes, the degrees of freedom (df) for the LRT (df=number of parameters of H_{full}−number of parameters of H_{reduced}), and the chromosome-wise type I error rate. A significance level of 5% chromosome wise was considered to be significant.

### Results BTA11

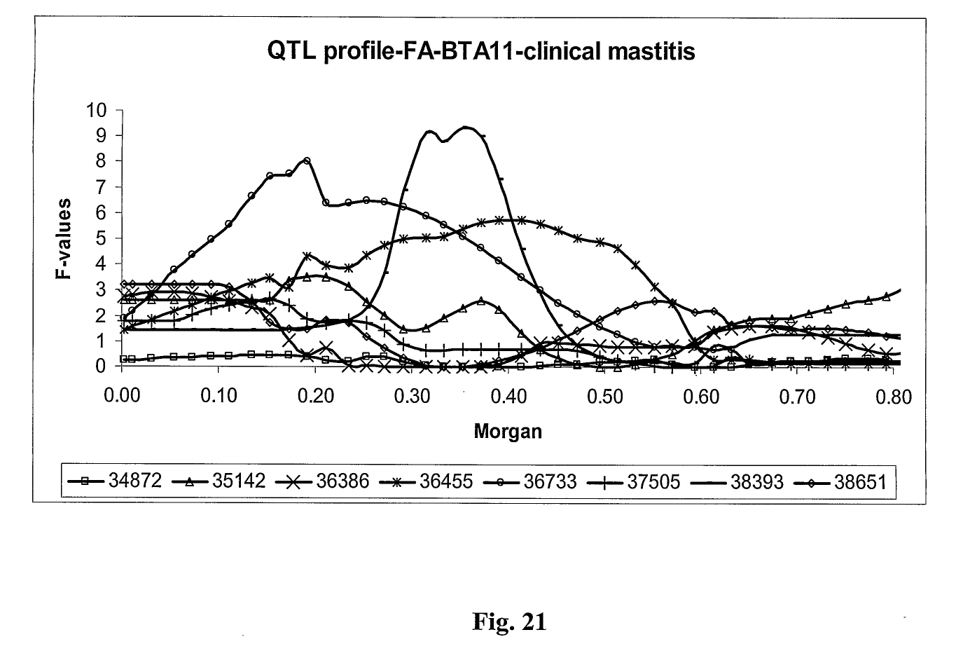

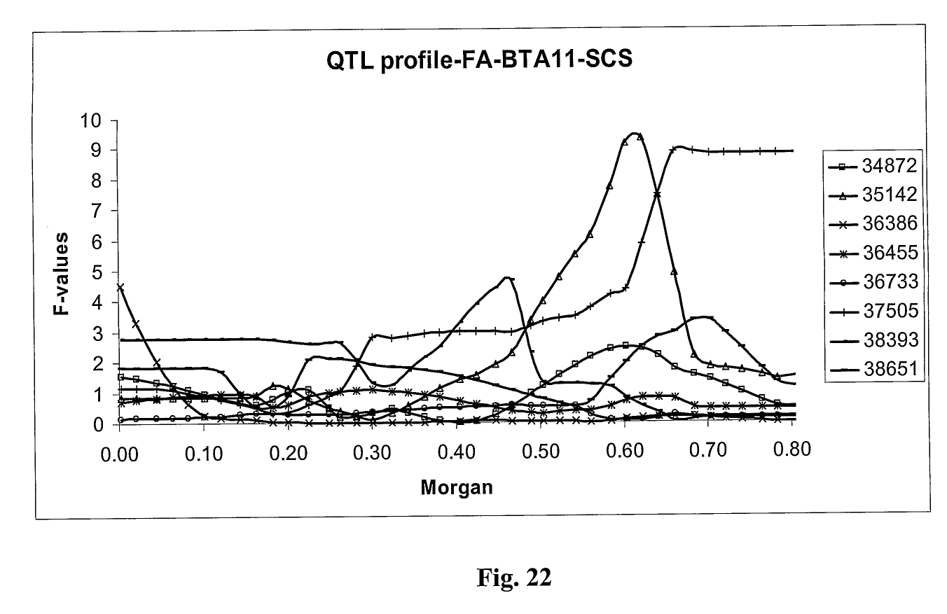

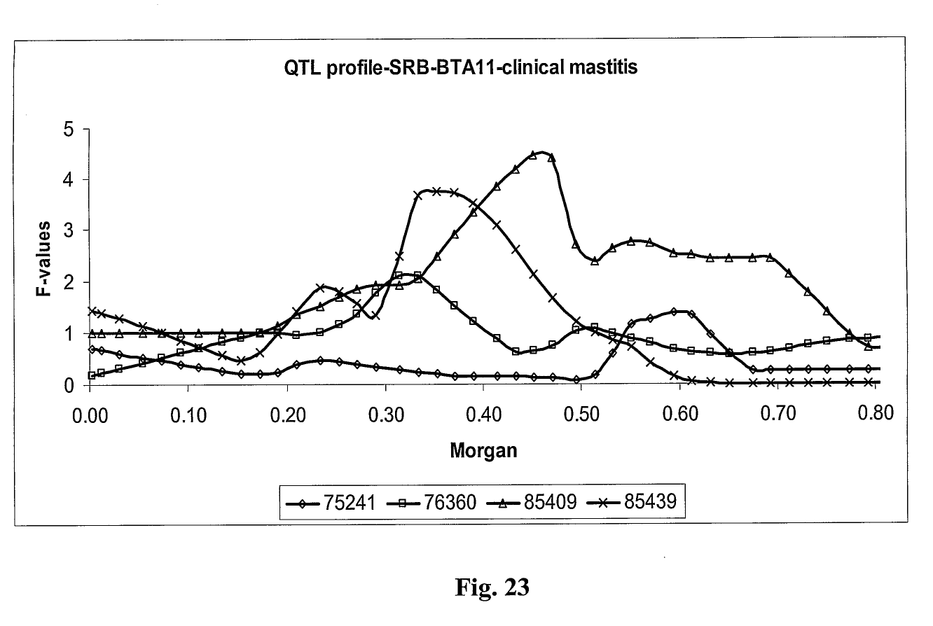

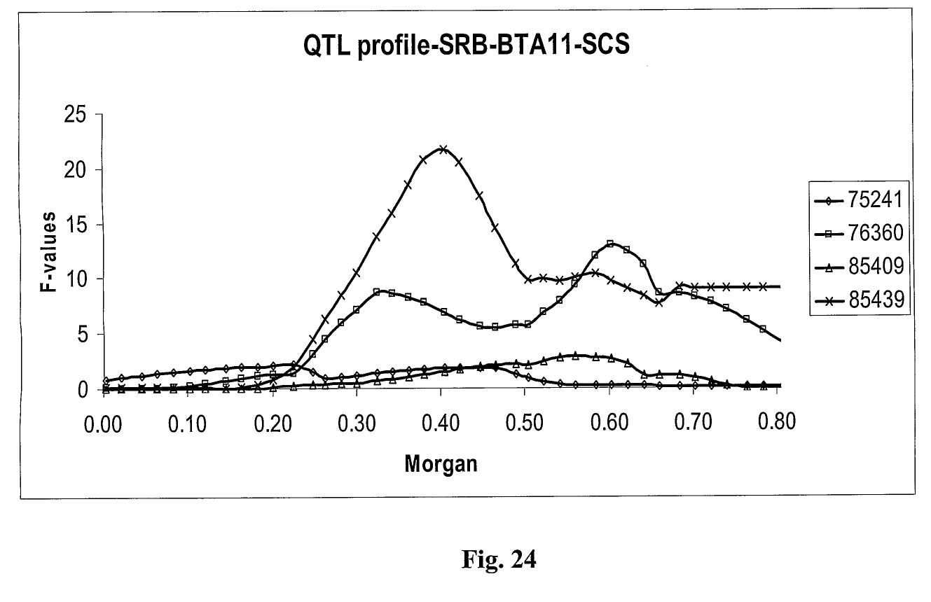

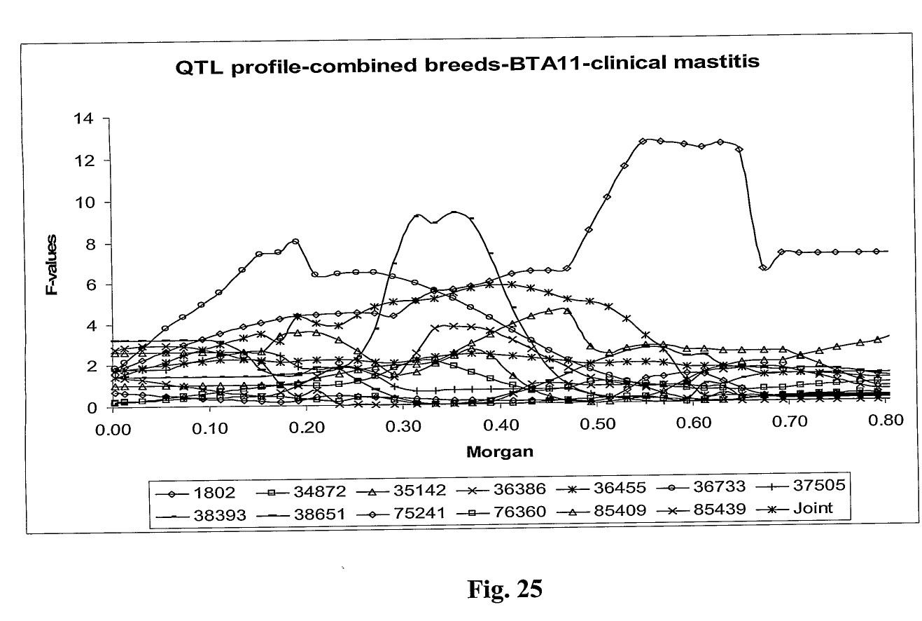

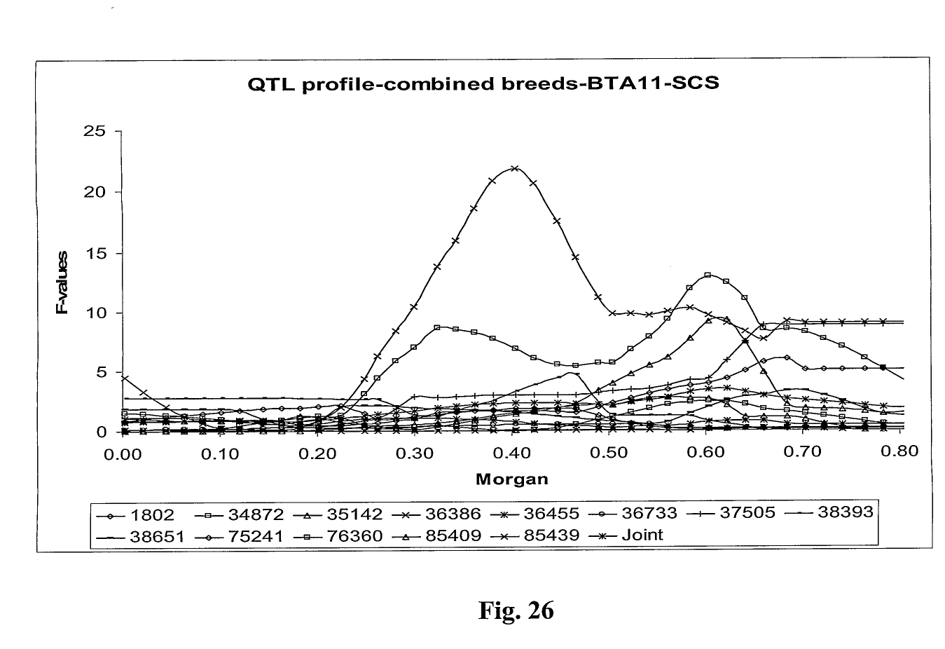

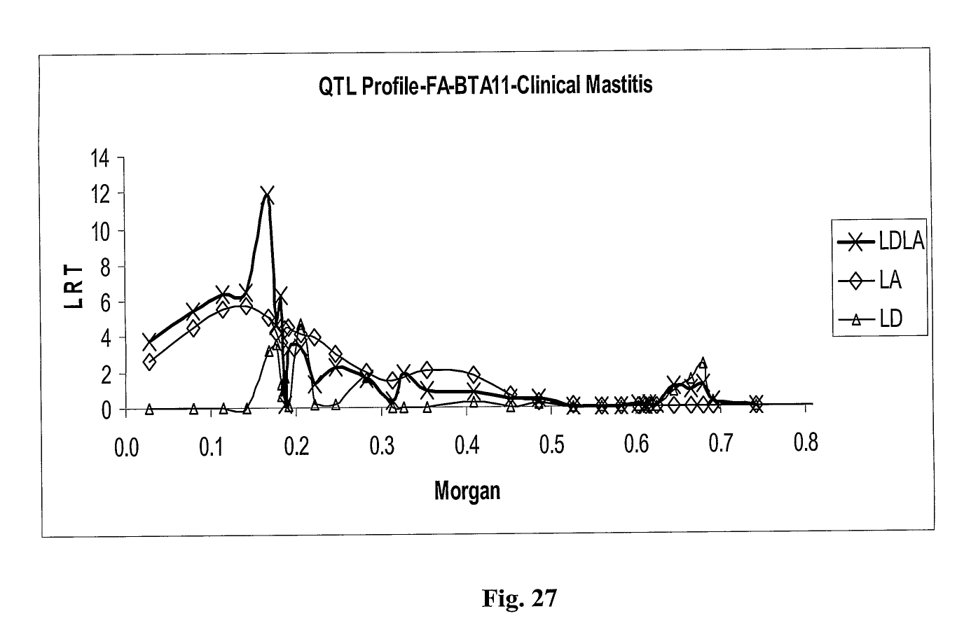

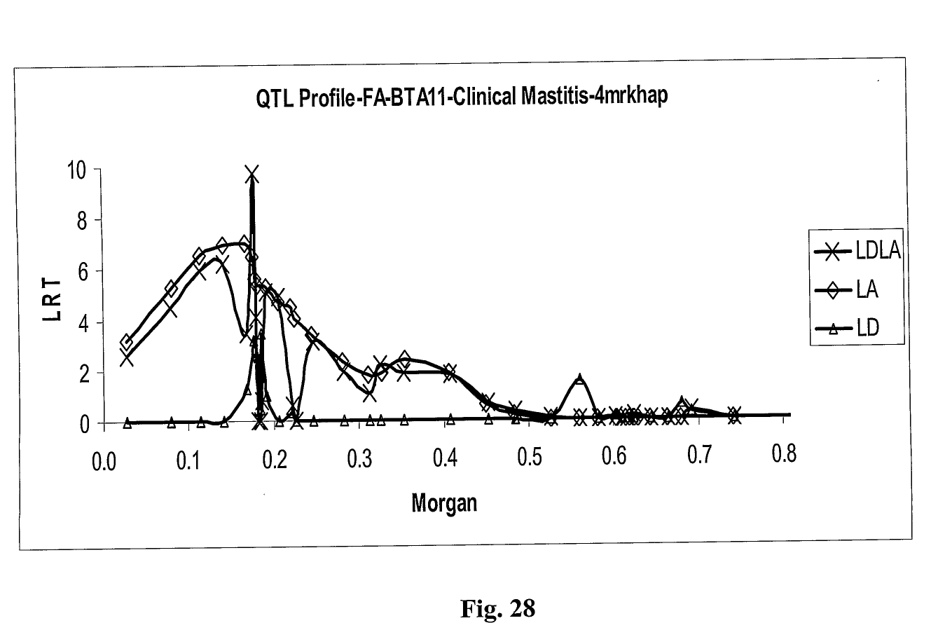

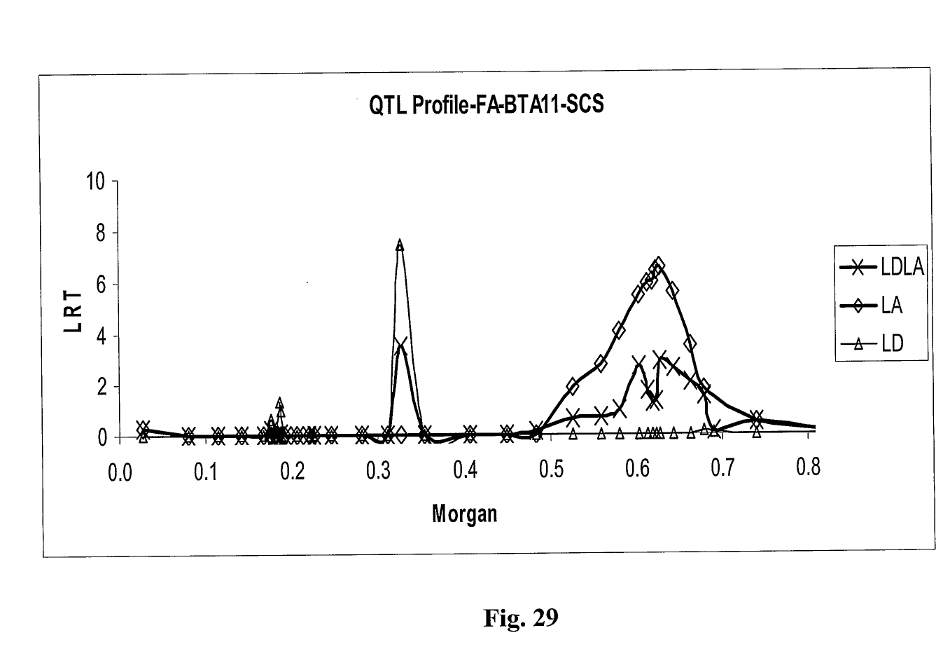

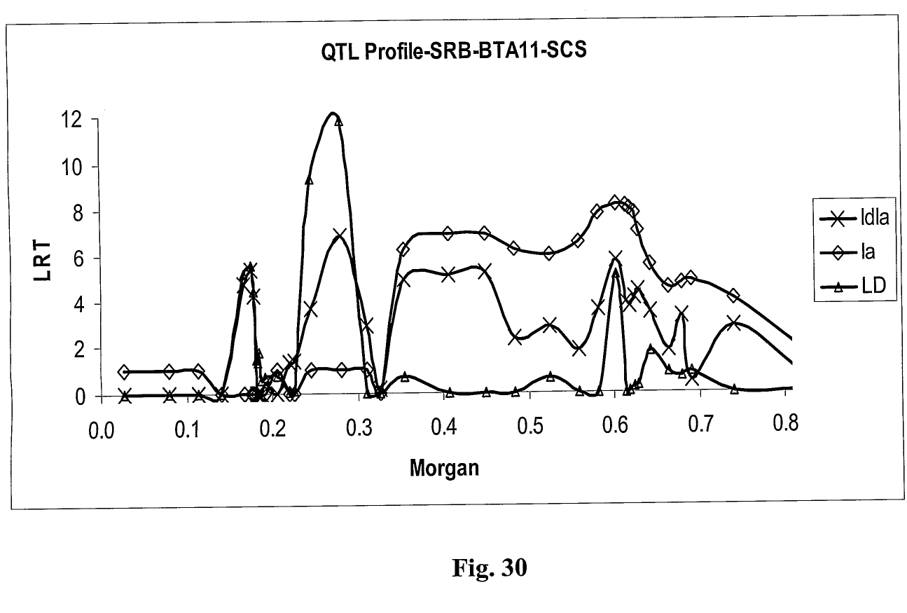

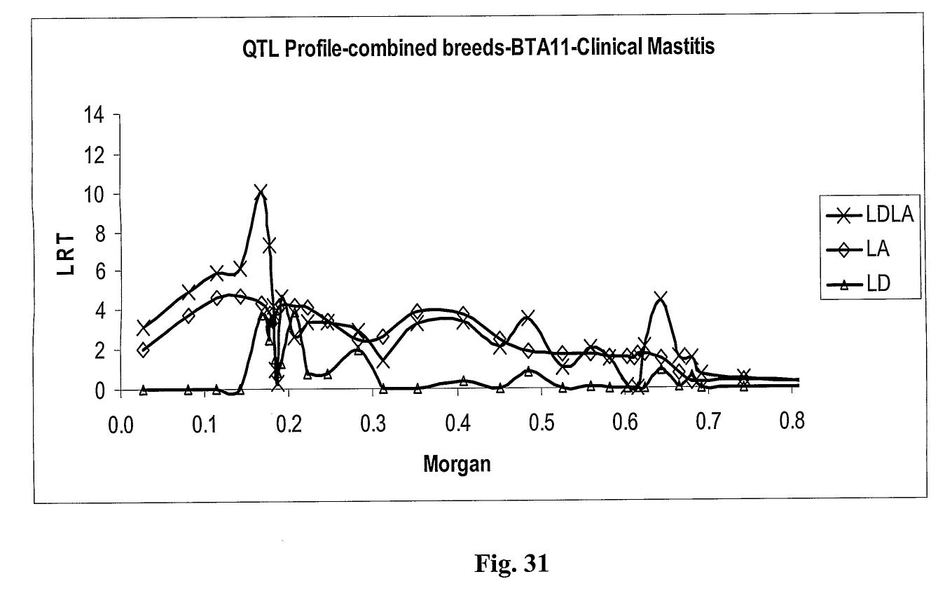

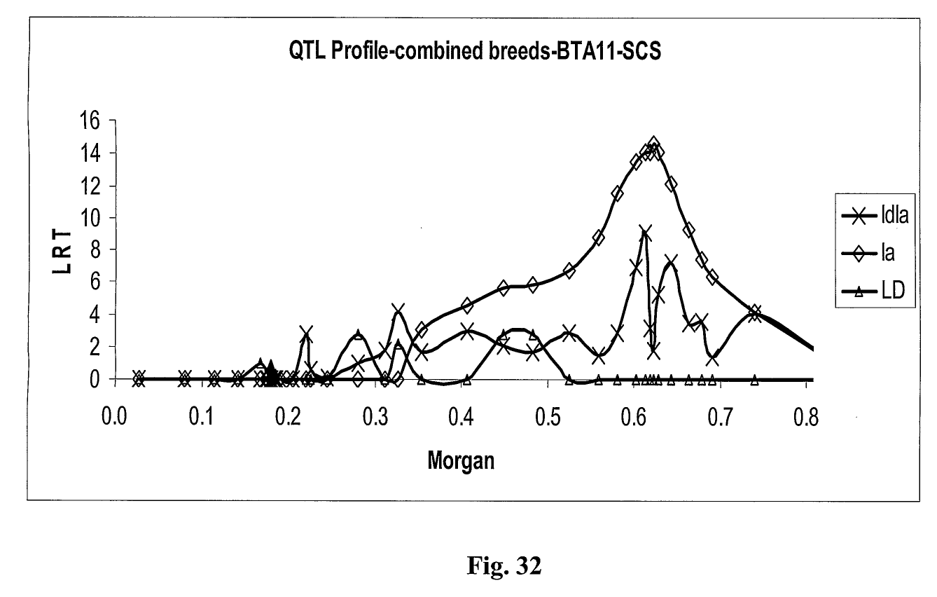

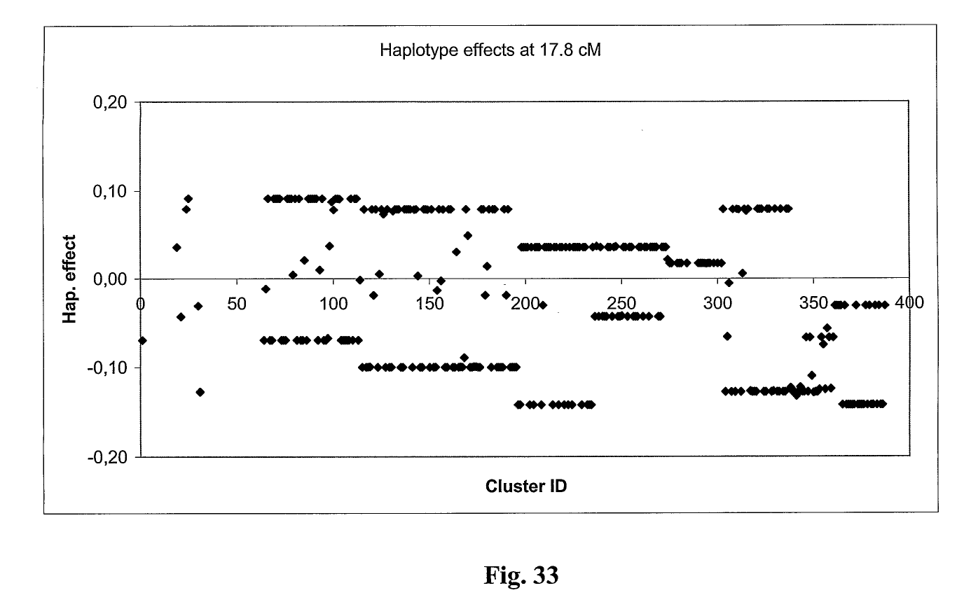

Table 57 shows the results from the regression analysis on BTA11. The results of LA analysis using variance component method are presented in Table 58; the LDLA results are presented in Table 59, and the LD analysis results in Table 60. FIG. 21 (FA), FIG. 23 (SRB) and FIG. 25 (Red combined) present the QTL graphs for the regression analysis for the trait clinical mastitis; and FIG. 22 (FA), FIG. 24 (SRB) and FIG. 26 (Red combined) present the QTL graphs for the trait somatic cell score. The variance component method was used to detect QTL across families QTL analysis. The QTL profiles obtained in variance component based method using LA, LDLA and LD for clinical mastitis are presented in FIG. 27 (FA), FIG. 28 (FA with a 4-marker window for IBD), and FIG. 31 (Red combined). The QTL profile in LA, LDLA and LD analysis for the trait SCS is presented in FIG. 29 (FA), FIG. 30 (SRB) and FIG. 32 (Red combined). The effect of large clusters on the trait clinical mastitis at the highest LDLA peak in Finnish Ayrshire with a 4-marker haplotype is presented in FIG. 33.

### Finnish Ayrshire

The analysis across of all eight Finnish Ayrshire (FA) half-sib family data for BTA11 using regression analysis resulted in 2 QTL which were significant at 5% level. The QTL affecting clinical mastitis was located at 11.3 cM and the QTL affecting somatic cell score was at 64.1 cM. One family was significant for mastitis QTL while two other families were reaching significant threshold. The QTL intervals in these three families were overlapping, though, spread over a large area. Two Finnish Ayrshire families were significant for the SCS QTL and the locations of the QTL in these two families were 6 cM apart. The across family linkage analysis for clinical mastitis using variance component method had the highest likelihood ratio test statistics (LRT) 5.74 at 14.2 cM. When combined linkage disequilibrium and linkage analysis (LDLA) was performed there was a sharp QTL peak at 16.8 cM with the LRT=11.82 between markers MNB-40 and AUP1. Though the highest LRT for LD analysis for clinical mastitis was at 20.6 cM between markers DIK4637 and UMBTL103, there was also some evidence for LD (LRT=3.18) between MNB-40 and AUP1. The part of clinical mastitis variance explained by the QTL at the highest LDLA peak was 15% of the total variance. By default 10-marker window was used to estimate the IBD probability. The LDLA analysis was repeated with a 4-marker window (FIG. 28). A sharp QTL peak (LRT=9.7) was observed at 17.8 cM between markers AUP1 and BM716. This interval between this 4-marker is 2.1 cM. Two Finnish Ayrshire sires families were segregating for the SCS QTL in the region 55-70 cM. The most probable QTL location was 64.1 cM in a multipoint regression analysis including all the FA families. The LA analysis with variance component has the highest LRT (6.6) at 62.8 cM. The QTL interval remained quite broad. The LDLA analysis failed to narrow down the QTL interval as no LD was observed in this region. The pleiotropic QTL model i.e. a QTL affecting both the traits clinical mastitis and SCS on BTA11 did not converge. The two linked QTL model put the clinical mastitis QTL at 14.2 cM and SCS QTL at 61.6 cM with LRT of 16.62. Therefore, it can be concluded that 2 QTL are segregating on BTA11 each one affecting one trait.

### Swedish Red and White

The multi-point regression analysis across Swedish Red and White (SRB) families revealed a QTL affecting SCS is segregating on BTA11 and the most probable location of the QTL is 61.2 cM. Two families were significant for the QTL. The probable location of the QTL in these two families was in 20 cM apart (59.8 and 40.4 cM). When across family linkage analysis was performed using variance component method in SRB the QTL interval for SCS was very large. The LDLA analysis could not make the QTL interval narrower due to lack of sufficient LD within the QTL interval. A 2-QTL model was ran to examine if the there were two linked QTL affecting SCS located between 30 to 70 cM region. A QTL at 61.2 cM affecting SCS was fixed and the region was scanned for another QTL affecting SCS. However, there was no evidence for the second QTL affecting SCS in this region. This QTL does not have pleiotropic effect on clinical mastitis in SRB cattle.

### Across Breed Analysis

Swedish Red and White breed is closely related with Nordic Ayrshire cattle breeds (Holmberg and Andersson-Eklund, 2004). The QTL on BTA11 affecting SCS was observed segregating in both FA (62.8 cM) and SRB (61.4 cM). Therefore, data from these two breeds were combined for QTL fine mapping on BTA11. One Danish Red (DR) family was observed segregating for QTL on BTA11 for clinical mastitis at 56.0 cM and for SCS at 66.9 cM. Danish Red cattle are also related historically with FA and SRB. Therefore, the DR family was included with the 13 FA and SRB families for joint analysis of BTA11. The across family linkage analysis for the trait clinical mastitis with variance component had highest LRT (4.72) at 14.2 cM. The reason for lower LA peak in joint Red data analysis than within FA analysis was due to inclusion of SRB and DR families, which do not segregate for the QTL at the proximal end of BTA11. The LDLA peak for Red combined data was at 16.8 cM (LRT=10.1). Though the highest evidence of LD was at 18.2 cM in Red data, but there was evidence of LD at the highest LDLA peak (LRT=3.8) between markers MNB-40 and AUP1.

The QTL affecting SCS in combined Red data analysis had a large interval (20 cM). The LRT in linkage analysis with variance component was 14.6 at 62.4. The highest LDLA peak was at 61.4 cM between markers MS2177 and HELMTT44. The reason for lower LRT in LDLA analysis than LA analysis was due to lack for LD within the SCS QTL interval. Though there was strong evidence of SCS QTL segregating on BTA11 from within and across family linkage analysis (both regression and variance component), the narrowing of the QTL location was not possible due to lack of LD within the QTL interval.

### Estimation of Haplotypes Effects

The LDLA analysis with a 4-marker window located the clinical mastitis QTL at 17.8 cM between markers AUP1 and BM716. Joint analysis of FA, SRB and one DR family showed that the QTL information at this location is primarily coming from Finnish Ayrshire families. Therefore, the clusters in the midpoint between the markers AUP1 and BM716 and their effects were studied in Finnish Ayrshire only. At the position 340 founder haplotypes coalesced to 63 clusters. There were eight clusters with frequency higher than 5% and the biggest cluster had the frequency 9.7%. One cluster with 32 haplotypes including two grandsire haplotypes had an estimated effect of −0.13 of phenotypic standard deviation.

The QTL fine mapping on BTA11 for the traits clinical mastitis and SCS confirmed that one QTL affecting clinical mastitis is segregating in Finnish Ayrshire cattle and one QTL affecting SCS is segregating in both Finnish Ayrshire and Swedish Red and White cattle. The LDLA analysis fine mapped the clinical mastitis to an interval of 2.1 cM. The QTL affecting SCS on BTA11 could not be fine mapped due to lack of linkage disequilibrium within the QTL interval.

### Haplotype Analysis

QTL fine mapping results mentioned above, points towards a QTL segregating for CM within the 4-marker region, MNB-40-AUP1-BM716-DIK2653. Therefore the clustering of founder haplotypes and haplotypes effects were studied at the midpoint between the markers AUP1 and BM716. QTL fine mapping results mentioned above, also points towards a QTL segregating for SCS within the 4-marker region, BM304-INRA177-UMBTL20-RM96 INRA177. Thus, the clustering of founder haplotypes and haplotypes effects were studied at the midpoint between the markers INRA177 and UMBTL20, respectively.

This was done within FA The haplotypes associated with high and low mastitis resistance were identified, se table 61 and table 62.

TABLE 57
With-family linkage analysis using regression interval analysis.

| Breed | Clinical Mastitis | | | | Somatic Cell Score | | | |
|---|---|---|---|---|---|---|---|---|
| and Sire | Position | F- | P- | | Position | F- | P- | |
| No. | (Morgan) | values | values* | Effect | (Morgan) | values | values* | Effect |

Finnish Ayrshire

| | | | | | | | | |
|---|---|---|---|---|---|---|---|---|
| 34872 | 0.262 | 0.49 | 0.03 | 0.077 | 0.627 | 2.44 | 0.52 | 0.22 |
| 35142 | 0.177 | 3.72 | 0.70 | −0.21 | 0.622 | 9.60 | 0.98 | −0.35 |
| 36386 | 0.045 | 2.94 | 0.65 | −0.34 | 0.00 | 4.63 | 0.82 | −0.31 |
| 36455 | 0.399 | 5.80 | 0.88 | −0.40 | 0.295 | 1.07 | 0.20 | −0.16 |
| 36733 | 0.187 | 8.11 | 0.94 | −0.45 | 0.485 | 0.55 | 0.07 | −0.13 |
| 37505 | 0.172 | 2.76 | 0.62 | −0.34 | 0.683 | 8.86 | 0.97 | 0.35 |
| 38393 | 0.352 | 9.38 | 0.97 | 0.37 | 0.461 | 4.62 | 0.83 | 0.43 |
| 38651 | 0.101 | 3.19 | 0.61 | −0.38 | 0.693 | 3.47 | 0.66 | −0.36 |
| Across- | 0.113 | 2.81 | 0.95 | | 0.641 | 2.67 | 0.95 | |
| family | | | | | | | | |

Swedish Red and White

| | | | | | | | | |
|---|---|---|---|---|---|---|---|---|
| 75241 | 0.593 | 1.39 | 0.32 | −0.16 | 0.224 | 2.14 | 0.51 | 0.17 |
| 76360 | 0.324 | 2.18 | 0.45 | 0.19 | 0.598 | 13.01 | 0.99 | 0.45 |
| 85409 | 0.461 | 4.57 | 0.86 | 0.45 | 0.565 | 2.80 | 0.62 | −0.32 |
| 85439 | 0.357 | 3.70 | 0.67 | −0.45 | 0.404 | 21.74 | 1.00 | −0.70 |
| 93907 | | | | | | | | |
| Across- | 0.410 | 2.29 | 0.64 | | 0.612 | 5.59 | 1.00 | |
| family | | | | | | | | |

Danish Red

| | | | | | | | | |
|---|---|---|---|---|---|---|---|---|
| 1802 | 0.560 | 12.64 | 0.99 | 0.57 | 0.669 | 6.07 | 0.91 | 0.42 |

*(1 − [p-value]) = chromosome wide significance level

TABLE 58
Summary of across-family linkage analysis (LA) using
variance component method

| | | Position | Peak LRT | |
|---|---|---|---|---|
| Breed | Trait | (Morgan) | statistics | Marker interval |
| Finish Ayrshire | CM | 0.142 | 5.74 | IL18RA-MNB-40 |
| (FA) | SCS | 0.628 | 6.56 | RM150-TGLA58 |
| Swedish Red and | CM | — | — | — |
| White (SRB) | SCS | 0.614 | 8.17 | MS2177-HELMTT44 |
| Red combined | CM | 0.142 | 4.72 | IL18RA-MNB-40 |
| | SCS | 0.624 | 14.57 | DIK5170-RM150 |

TABLE 59
Summary of linkage disequilibrium and linkage analysis
(LDLA) using variance component method

| | | Position | Peak LRT | |
|---|---|---|---|---|
| Breed | Trait | (Morgan) | statistics | Marker interval |
| Finish Ayrshire | CM | 0.168 | 11.82 | MNB-40-AUP1 |
| (FA) | SCS | 0.628 | 2.87 | RM150-TGLA58 |
| Swedish Red and | CM | — | — | — |
| White (SRB) | SCS | 0.603 | 5.72 | MB110-MS2177 |
| Red combined | CM | 0.168 | 10.06 | MNB-40-AUP1 |
| | SCS | 0.614 | 9.11 | MS2177-HELMTT44 |

TABLE 60
Summary of across-family linkage disequilibrium (LD)
analysis using variance component method

| | | Position | Peak LRT | |
|---|---|---|---|---|
| Breed | Trait | (Morgan) | statistics | Marker interval |
| Finish Ayrshire | CM | 0.206 | 4.72 | DIK4637-UMBTL103 |
| (FA) | SCS | 0.327 | 7.48 | INRA177-UMBTL20 |
| Swedish Red and | CM | — | — | — |
| White (SRB) | SCS | 0.281 | 11.82 | BM2818-BM304 |
| Red combined | CM | 0.206 | 3.89 | DIK4637-UMBTL103 |
| | SCS | 0.483 | 2.75 | BM7169-BMS1716 |

TABLE 61
BTA11, 17.8 cM (6^{th} interval), CM, FA

| Allele/Allele combination | | No. | |
|---|---|---|---|
| (marker MNB- | | grandsire | |
| 40-AUP1-BM716- | Total no. of | allele in the | |
| DIK2653) | founder haplotypes | haplotype | Effect |
| 294/296/300/304-10- | 33 | 1 | −0.0695 |
| 171-238 | | | |
| 294/298/302/304-10- | 32 | 2 | +0.0792 |
| 175-238 | | | |
| 292/294-30-167-244 | 30 | 2 | −0.1275 |
| 292/304-30-173-238 | 28 | 2 | +0.0357 |
| 290/302-10-167-240 | 23 | 1 | +0.0916 |
| 290/294/298/300/302 | 20 | 1 | −0.0310 |
| 298/300-30-167-256 | 19 | 1 | −0.0431 |

TABLE 62
BTA11, 32.65 cM (19^{th} interval), SCS, FA - 4 marker-hap

| Allele/Allele combination | | No. | |
|---|---|---|---|
| (marker | | grandsire | |
| BM304-INRA177- | Total no. of | allele in the | |
| UMBTL20-RM96) | founder haplotypes | haplotype | Effect |
| 105/109/115/123-99- | 82 | 2 | −0.1401 |
| 224-130/124 | | | |
| 105/109/115/123-93- | 35 | 2 | −0.0189 |
| 228-124/134 | | | |
| 105/123-93-220- | 33 | 3 | +0.1658 |
| 124/134 | | | |
| 109/115/123-93-236- | 31 | 1 | +0.0939 |
| 124/130 | | | |
| 123-101-238-124 | 24 | 1 | −0.0900 |
| 105/123-97-236- | 19 | 2 | −0.0458 |
| 120/124/130 | | | |

1. A method for determining the resistance to mastitis in a bovine subject, comprising detecting in a sample from said bovine subject the presence or absence of at least one genetic marker that is linked to at least one trait indicative of mastitis resistance, wherein said at least one genetic marker is located on the bovine chromosome BTA9 in the region flanked by and including the polymorphic microsatellite markers C6orf93 and inra084 and/or BTA11 in the region flanked by and including the polymorphic microsatellite markers HELMTT43 and BM3501, wherein the presence or absence of said at least one genetic marker is indicative of mastitis resistance of said bovine subject or off-spring therefrom.

2. The method according to claim 1 for determining the resistance to mastitis in a bovine subject, comprising detecting in a sample from said bovine subject the presence or absence of at least one genetic marker that is linked to at least one trait indicative of mastitis resistance, wherein said at least one genetic marker is located on the bovine chromosome BTA9 in the region flanked by and including the polymorphic microsatellite markers C6orf93 and inra084, wherein the presence or absence of said at least one genetic marker is indicative of mastitis resistance of said bovine subject or off-spring therefrom.

3. The method according to claim 1, wherein the at least one genetic marker is linked to a bovine trait for resistance to mastitis.

4. (canceled)

5. The method according to claim 1, wherein the at least one genetic marker is located in the region flanked by and including the genetic markers bms2251 and inra084 of BTA9.

6. The method according to claim 1, wherein the at least one genetic marker is located in the region flanked by and including the genetic markers bm7234 and inra144 of BTA9.

7. The method according to claim 1, wherein the at least one genetic marker is located in the region flanked by and including the genetic markers bms2819 and inra144 of BTA9.

8. The method according to claim 1, wherein the at least one genetic marker is located in the region flanked by and including the genetic markers bms2251 and inra144 of BTA9.

9. The method according to claim 1, wherein the at least one genetic marker is located in the region flanked by and including the genetic markers bms2819 and inra084 of BTA9.

10. The method according to claim 1, wherein the at least one genetic marker is located in the region flanked by and including the genetic markers bm7234 and bms2819 of BTA9.

11. The method according to claim 1, wherein the at least one genetic marker is located in the region flanked by and including the genetic markers bm7234 and bm4208 of BTA9.

12. The method according to claim 1, wherein the at least one genetic marker is located in the region flanked by and including the genetic markers inra144 and rgs17 of BTA9.

13-14. (canceled)

15. The method according to claim 1, wherein the at least one genetic marker is located in the region flanked by and including the genetic markers bm4208 and inra144.

16-32. (canceled)

33. The method according to claim 1, wherein the at least one genetic marker is located in the region from 69.35 cM to 74.5 cM of BTA9.

34-40. (canceled)

41. The method according to claim 1 for determining the resistance to mastitis in a bovine subject, wherein at least one genetic marker is located on the bovine chromosome 11 in the region flanked by and including the polymorphic microsatellite markers HELMTT43 and BM3501, wherein the at least one genetic marker is linked to mastitis resistance, said method comprising detecting in a genetic material from said subject the presence or absence of at least one genetic marker that is linked to at least one trait indicative of mastitis resistance, wherein the presence of said at least one genetic marker is indicative of displaying mastitis resistance and/or producing off-spring displaying mastitis resistance.

42. (canceled)

43. The method according to claim 1, wherein the at least one genetic marker is located in the region flanked by and including the genetic markers HELMTT43 and MNB-70 of BTA11.

44. The method according to claim 1, wherein the at least one genetic marker is located in the region flanked by and including the genetic markers MNB-40 and MNB-70 of BTA11.

45-56. (canceled)

57. The method according to claim 1, wherein the at least one genetic marker is located in the region flanked by and including the genetic markers BMS2325 and DIK4637.

58-70. (canceled)

71. The method according to claim 1, wherein the at least one genetic marker is located in the region from 2.249 cM to 97.223 cM of BTA11.

72. (canceled)

73. The method according to claim 1, wherein the at least one genetic marker is located in the region from 19.440 cM to 23.829 cM of BTA11.

74-79. (canceled)

80. A diagnostic kit for use in detecting the presence or absence in a bovine subject of at least one genetic marker associated with resistance to mastitis, comprising at least one oligonucleotide sequence selected from the group consisting of SEQ ID NO.: 1 to SEQ ID NO.: 192 and combinations thereof.


SEQUENCE LISTING (192 sequences)

SEQ ID NO: 1 - DNA, 20 bp, Artificial
  Primer
aacggctttcactttcttgc

SEQ ID NO: 2 - DNA, 18 bp, Artificial
  Primer
ctgggtgaacaaatgggc

SEQ ID NO: 3 - DNA, 22 bp, Artificial
  Primer
gatcaccttgccactatttcct

SEQ ID NO: 4 - DNA, 21 bp, Artificial
  Primer
acatgacagccagctgctact

SEQ ID NO: 5 - DNA, 19 bp, Artificial
  Primer
cagctttccatcccctttc

SEQ ID NO: 6 - DNA, 20 bp, Artificial
  Primer
ctcccatcccaaacacagac

SEQ ID NO: 7 - DNA, 20 bp, Artificial
  Primer
ttgaccctgaaagatgtcca

SEQ ID NO: 8 - DNA, 21 bp, Artificial
  Primer
cacggtttatcagcttgggta

SEQ ID NO: 9 - DNA, 24 bp, Artificial
  Primer
aaatggaggtaatgaaataaaata

SEQ ID NO: 10 - DNA, 21 bp, Artificial
  Primer
caaacccatggactgtaacct

SEQ ID NO: 11 - DNA, 20 bp, Artificial
  Primer
actttcagttttgggctgac

SEQ ID NO: 12 - DNA, 20 bp, Artificial
  Primer
tgtcactaggtaaattggtg

SEQ ID NO: 13 - DNA, 22 bp, Artificial
  Primer
ttctgcaatgttgagcttcaag

SEQ ID NO: 14 - DNA, 23 bp, Artificial
  Primer
gatctgaaaaagaaatgaataga

SEQ ID NO: 15 - DNA, 18 bp, Artificial
  Primer
tgggaaagttggcaaatg

SEQ ID NO: 16 - DNA, 22 bp, Artificial
  Primer
ttgtgatacctgaaatggtcaa

SEQ ID NO: 17 - DNA, 22 bp, Artificial
  Primer
ggaaagagtaggtgattccctg

SEQ ID NO: 18 - DNA, 22 bp, Artificial
  Primer
atttaattgtcatcccaggtga

SEQ ID NO: 19 - DNA, 22 bp, Artificial
  Primer
ttaaagcaatttagggagctta

SEQ ID NO: 20 - DNA, 22 bp, Artificial
  Primer
ctagtatctaaaatgaacagaa

SEQ ID NO: 21 - DNA, 20 bp, Artificial
  Primer
tgggtaagtgggaaaggatg

SEQ ID NO: 22 - DNA, 20 bp, Artificial
  Primer
ctcagccaggttgtcctctc

SEQ ID NO: 23 - DNA, 21 bp, Artificial
  Primer
caggtacacagtaaagacaga

SEQ ID NO: 24 - DNA, 20 bp, Artificial
  Primer
ctgctttgggggcacagtca

SEQ ID NO: 25 - DNA, 20 bp, Artificial
  Primer
ataagggtgcactggcagaa

SEQ ID NO: 26 - DNA, 20 bp, Artificial
  Primer
gcagtccaggggattgtaaa

SEQ ID NO: 27 - DNA, 20 bp, Artificial
  Primer
agtgcctggcaggtattgaa

SEQ ID NO: 28 - DNA, 20 bp, Artificial
  Primer
cctcggtttcccaatcatta

SEQ ID NO: 29 - DNA, 24 bp, Artificial
  Primer
gtagttatcaaaataagaatgctt

SEQ ID NO: 30 - DNA, 24 bp, Artificial
  Primer
tatgttttccttttggttgaatag

SEQ ID NO: 31 - DNA, 20 bp, Artificial
  Primer
tctgaaacctggaggaggag

SEQ ID NO: 32 - DNA, 20 bp, Artificial
  Primer
gaaacttccaccaccctcaa

SEQ ID NO: 33 - DNA, 20 bp, Artificial
  Primer
cctctgaaaccccagacttg

SEQ ID NO: 34 - DNA, 22 bp, Artificial
  Primer
aaaaacccaaaacaacacacaa

SEQ ID NO: 35 - DNA, 21 bp, Artificial
  Primer
tccccaaattctgatgactct

SEQ ID NO: 36 - DNA, 21 bp, Artificial
  Primer
tcagcccttaaaacgtaagca

SEQ ID NO: 37 - DNA, 25 bp, Artificial
  Primer
tcaaatctcattctctccagaaggc

SEQ ID NO: 38 - DNA, 25 bp, Artificial
  Primer
ccaacttatattaggcacaatgtcc

SEQ ID NO: 39 - DNA, 20 bp, Artificial
  Primer
cttgatccttatagaactgg

SEQ ID NO: 40 - DNA, 20 bp, Artificial
  Primer
acacaaaatcagatcagtgg

SEQ ID NO: 41 - DNA, 25 bp, Artificial
  Primer
ccttctctgaatttttgttgaaagc

SEQ ID NO: 42 - DNA, 22 bp, Artificial
  Primer
ggacagaagtgagtgactgaga

SEQ ID NO: 43 - DNA, 24 bp, Artificial
  Primer
ttaaatgggaccagataaatagga

SEQ ID NO: 44 - DNA, 24 bp, Artificial
  Primer
aaatgagaaccagataagcctaaa

SEQ ID NO: 45 - DNA, 20 bp, Artificial
  Primer
aagaagtagagcgggggaag

SEQ ID NO: 46 - DNA, 20 bp, Artificial
  Primer
gaatgccaagcatcccttac

SEQ ID NO: 47 - DNA, 23 bp, Artificial
  Primer
ttgcactgatctctgctaaagtg

SEQ ID NO: 48 - DNA, 20 bp, Artificial
  Primer
tctccccacaacatcattca

SEQ ID NO: 49 - DNA, 20 bp, Artificial
  Primer
ggaaagacaagagggtgctg

SEQ ID NO: 50 - DNA, 21 bp, Artificial
  Primer
tgttgcaaaaagcaaatttca

SEQ ID NO: 51 - DNA, 23 bp, Artificial
  Primer
catgatatttaccctgtgtgtgc

SEQ ID NO: 52 - DNA, 20 bp, Artificial
  Primer
gaggagctggagggctaaag

SEQ ID NO: 53 - DNA, 21 bp, Artificial
  Primer
gggtaggagcttttgtaggtg

SEQ ID NO: 54 - DNA, 22 bp, Artificial
  Primer
gccatcacccttctcttatatg

SEQ ID NO: 55 - DNA, 20 bp, Artificial
  Primer
atgactcctggagcagaacc

SEQ ID NO: 56 - DNA, 25 bp, Artificial
  Primer
gaagagtaagctgtatttttcatgc

SEQ ID NO: 57 - DNA, 24 bp, Artificial
  Primer
acttgttaggagggctattgttaa

SEQ ID NO: 58 - DNA, 22 bp, Artificial
  Primer
ccacatacaccaccaacattaa

SEQ ID NO: 59 - DNA, 24 bp, Artificial
  Primer
ttggcacttactacctcatatgtt

SEQ ID NO: 60 - DNA, 22 bp, Artificial
  Primer
ttttctggatgttgagcctatt

SEQ ID NO: 61 - DNA, 20 bp, Artificial
  Primer
gagaatcacctagagaggca

SEQ ID NO: 62 - DNA, 24 bp, Artificial
  Primer
ctttctctttaaattctatatggt

SEQ ID NO: 63 - DNA, 22 bp, Artificial
  Primer
tcaaaaagttggacatgactga

SEQ ID NO: 64 - DNA, 24 bp, Artificial
  Primer
aggttttcaaatgagagacttttc

SEQ ID NO: 65 - DNA, 20 bp, Artificial
  Primer
ggagggtgtgcttgaaagag

SEQ ID NO: 66 - DNA, 20 bp, Artificial
  Primer
gctggcgttctctctcgtat

SEQ ID NO: 67 - DNA, 20 bp, Artificial
  Primer
gacttgccccaatcctactg

SEQ ID NO: 68 - DNA, 21 bp, Artificial
  Primer
atttcaggtttgttggttccc

SEQ ID NO: 69 - DNA, 21 bp, Artificial
  Primer
tggtgctctgggaacatagac

SEQ ID NO: 70 - DNA, 20 bp, Artificial
  Primer
atcacagtggcctgaacaca

SEQ ID NO: 71 - DNA, 21 bp, Artificial
  Primer
ttttctgctcatgcttcagtg

SEQ ID NO: 72 - DNA, 22 bp, Artificial
  Primer
gcaggctatagtccatgacatc

SEQ ID NO: 73 - DNA, 21 bp, Artificial
  Primer
gggttctgtttgcttttcttc

SEQ ID NO: 74 - DNA, 22 bp, Artificial
  Primer
ctagcactggcaggtagattct

SEQ ID NO: 75 - DNA, 20 bp, Artificial
  Primer
ctcggtgatgtttttgctga

SEQ ID NO: 76 - DNA, 20 bp, Artificial
  Primer
cgccccagctctttctagtt

SEQ ID NO: 77 - DNA, 20 bp, Artificial
  Primer
gggatgaacattgagggttg

SEQ ID NO: 78 - DNA, 20 bp, Artificial
  Primer
catgatcaagatgggggaag

SEQ ID NO: 79 - DNA, 20 bp, Artificial
  Primer
caagacaggtgtttcaatct

SEQ ID NO: 80 - DNA, 21 bp, Artificial
  Primer
atcgactctgggggatgatgt

SEQ ID NO: 81 - DNA, 21 bp, Artificial
  Primer
ttttgcgagtccagttaaaca

SEQ ID NO: 82 - DNA, 20 bp, Artificial
  Primer
ggaaaagccagagcaaaatg

SEQ ID NO: 83 - DNA, 20 bp, Artificial
  Primer
ttcctaggcctgtgctcatt

SEQ ID NO: 84 - DNA, 20 bp, Artificial
  Primer
tggaccaggcataaggattt

SEQ ID NO: 85 - DNA, 20 bp, Artificial
  Primer
aacggctttcactttcttgc

SEQ ID NO: 86 - DNA, 18 bp, Artificial
  Primer
ctgggtgaacaaatgggc

SEQ ID NO: 87 - DNA, 16 bp, Artificial
  Primer
gcggccgcgttgagag

SEQ ID NO: 88 - DNA, 24 bp, Artificial
  Primer
ttccactttatgatgagcaggttc

SEQ ID NO: 89 - DNA, 23 bp, Artificial
  Primer
ttcactgattgtcattccctaga

SEQ ID NO: 90 - DNA, 24 bp, Artificial
  Primer
taagcaaataaatggtgctagtca

SEQ ID NO: 91 - DNA, 20 bp, Artificial
  Primer
tcagtacactggccaccatg

SEQ ID NO: 92 - DNA, 20 bp, Artificial
  Primer
cactgcatgcttttccaaac

SEQ ID NO: 93 - DNA, 21 bp, Artificial
  Primer
gctcacaggttctgaggactc

SEQ ID NO: 94 - DNA, 22 bp, Artificial
  Primer
aacttgaagaaggaatgctgag

SEQ ID NO: 95 - DNA, 22 bp, Artificial
  Primer
tcggtgtgggaggtgactacat

SEQ ID NO: 96 - DNA, 20 bp, Artificial
  Primer
tgctggtgggctccgtcacc

SEQ ID NO: 97 - DNA, 20 bp, Artificial
  Primer
ctaaagctttcctccatctc

SEQ ID NO: 98 - DNA, 20 bp, Artificial
  Primer
cctggtgatgtttggatgtc

SEQ ID NO: 99 - DNA, 24 bp, Artificial
  Primer
catgaaacacaaacataaatggga

SEQ ID NO: 100 - DNA, 23 bp, Artificial
  Primer
gggaccaaaaatacatcacagta

SEQ ID NO: 101 - DNA, 19 bp, Artificial
  Primer
gctgctggagatgctggat

SEQ ID NO: 102 - DNA, 21 bp, Artificial
  Primer
tgattcacgtcctctggaggt

SEQ ID NO: 103 - DNA, 18 bp, Artificial
  Primer
gctctggtgacccaggtg

SEQ ID NO: 104 - DNA, 20 bp, Artificial
  Primer
ctggcaggagatgagaggag

SEQ ID NO: 105 - DNA, 21 bp, Artificial
  Primer
tgcatcaactaagccacactg

SEQ ID NO: 106 - DNA, 20 bp, Artificial
  Primer
ttactcgctggttttctggg

SEQ ID NO: 107 - DNA, 20 bp, Artificial
  Primer
cgagttcgaggccatgtgaa

SEQ ID NO: 108 - DNA, 17 bp, Artificial
  Primer
cggagcagggagagggt

SEQ ID NO: 109 - DNA, 20 bp, Artificial
  Primer
ctgttctattccctatactg

SEQ ID NO: 110 - DNA, 21 bp, Artificial
  Primer
attatcatgcatacactttga

SEQ ID NO: 111 - DNA, 20 bp, Artificial
  Primer
cagggtgacagcggcgggcc

SEQ ID NO: 112 - DNA, 27 bp, Artificial
  Primer
gaagtaccgagtttattttcaacaaat

SEQ ID NO: 113 - DNA, 24 bp, Artificial
  Primer
caagaccggcctgagctacaagag

SEQ ID NO: 114 - DNA, 23 bp, Artificial
  Primer
gtgcggtggatgagtggggacag

SEQ ID NO: 115 - DNA, 21 bp, Artificial
  Primer
atcagtcgttcccagaatgtc

SEQ ID NO: 116 - DNA, 22 bp, Artificial
  Primer
ttgatatcctctctgtcaagcc

SEQ ID NO: 117 - DNA, 22 bp, Artificial
  Primer
gggcagatgtgagtaattttcc

SEQ ID NO: 118 - DNA, 21 bp, Artificial
  Primer
aactgagctgtatggtggacg

SEQ ID NO: 119 - DNA, 25 bp, Artificial
  Primer
ggttacagtccatgagtttgcaaag

SEQ ID NO: 120 - DNA, 21 bp, Artificial
  Primer
acagaggtggggtagactttt

SEQ ID NO: 121 - DNA, 21 bp, Artificial
  Primer
ggagctacggagtcaccatgt

SEQ ID NO: 122 - DNA, 21 bp, Artificial
  Primer
gtaggtccagcaatcgctcat

SEQ ID NO: 123 - DNA, 25 bp, Artificial
  Primer
caaagagtgggtctcaacatgaatc

SEQ ID NO: 124 - DNA, 15 bp, Artificial
  Primer
gggctgggcctgctc

SEQ ID NO: 125 - DNA, 22 bp, Artificial
  Primer
cagaagtcttgcctgggaagtc

SEQ ID NO: 126 - DNA, 19 bp, Artificial
  Primer
ccgtgtctgcctcttgtga

SEQ ID NO: 127 - DNA, 22 bp, Artificial
  Primer
cagcctccttcatactccttct

SEQ ID NO: 128 - DNA, 20 bp, Artificial
  Primer
ggggaagggagcagattgta

SEQ ID NO: 129 - DNA, 20 bp, Artificial
  Primer
ccctgtcctgacgtctgttt

SEQ ID NO: 130 - DNA, 20 bp, Artificial
  Primer
cacaaccaagggaaaaggaa

SEQ ID NO: 131 - DNA, 21 bp, Artificial
  Primer
agtacttggcttgctttgctc

SEQ ID NO: 132 - DNA, 22 bp, Artificial
  Primer
ttaaatttccatctcaccctgg

SEQ ID NO: 133 - DNA, 20 bp, Artificial
  Primer
atggccgtccattcagatac

SEQ ID NO: 134 - DNA, 20 bp, Artificial
  Primer
cctccctgtggtttatggaa

SEQ ID NO: 135 - DNA, 18 bp, Artificial
  Primer
agagaggccaaagctggg

SEQ ID NO: 136 - DNA, 19 bp, Artificial
  Primer
tttccttgggcttcaggag

SEQ ID NO: 137 - DNA, 20 bp, Artificial
  Primer
tccatcttgcagaagtgtgc

SEQ ID NO: 138 - DNA, 19 bp, Artificial
  Primer
agggccaggaatgctagtg

SEQ ID NO: 139 - DNA, 23 bp, Artificial
  Primer
tgctgtaggagaaaataaagcag

SEQ ID NO: 140 - DNA, 21 bp, Artificial
  Primer
tttgctgagaggactttgaga

SEQ ID NO: 141 - DNA, 21 bp, Artificial
  Primer
tgtgctctaaagcttgacctg

SEQ ID NO: 142 - DNA, 20 bp, Artificial
  Primer
tcagctggttgagggttctc

SEQ ID NO: 143 - DNA, 21 bp, Artificial
  Primer
tctccttcatagctggcatct

SEQ ID NO: 144 - DNA, 21 bp, Artificial
  Primer
ttggatggcatcactgacttg

SEQ ID NO: 145 - DNA, 21 bp, Artificial
  Primer
ccaaatgatggttcaagtttg

SEQ ID NO: 146 - DNA, 22 bp, Artificial
  Primer
gctcatgataaagggaattcag

SEQ ID NO: 147 - DNA, 20 bp, Artificial
  Primer
taatgagcagacccacacag

SEQ ID NO: 148 - DNA, 20 bp, Artificial
  Primer
accattggctctcctaggtc

SEQ ID NO: 149 - DNA, 22 bp, Artificial
  Primer
ttctgtggttgaagagtgttcc

SEQ ID NO: 150 - DNA, 21 bp, Artificial
  Primer
caatggctaagaggtccagtg

SEQ ID NO: 151 - DNA, 23 bp, Artificial
  Primer
ctggtgttcctttcatatcaacc

SEQ ID NO: 152 - DNA, 23 bp, Artificial
  Primer
ggcacgtactaacctgtaaaacc

SEQ ID NO: 153 - DNA, 23 bp, Artificial
  Primer
tccaaaagtttcgtgacatattg

SEQ ID NO: 154 - DNA, 20 bp, Artificial
  Primer
caccaggcttctctgttgaa

SEQ ID NO: 155 - DNA, 21 bp, Artificial
  Primer
ttccatgtcacagatagcctc

SEQ ID NO: 156 - DNA, 21 bp, Artificial
  Primer
acattatcacaagacaccagc

SEQ ID NO: 157 - DNA, 22 bp, Artificial
  Primer
tcgcaaaaagttggacaagact

SEQ ID NO: 158 - DNA, 21 bp, Artificial
  Primer
ttagcagggtgcctgacactt

SEQ ID NO: 159 - DNA, 22 bp, Artificial
  Primer
ggtaaaatcctgcaaaacacag

SEQ ID NO: 160 - DNA, 22 bp, Artificial
  Primer
tgactgtatagactgaagcaac

SEQ ID NO: 161 - DNA, 22 bp, Artificial
  Primer
tggtatgtagttacagcagccc

SEQ ID NO: 162 - DNA, 22 bp, Artificial
  Primer
ccattgaaacagacatgaatgc

SEQ ID NO: 163 - DNA, 20 bp, Artificial
  Primer
gtgggttggagaggtacaag

SEQ ID NO: 164 - DNA, 21 bp, Artificial
  Primer
agaaatggccttgagaaagag

SEQ ID NO: 165 - DNA, 22 bp, Artificial
  Primer
gtgtctgtcaaaagatgaatgg

SEQ ID NO: 166 - DNA, 20 bp, Artificial
  Primer
gacaactgcttctcgttggg

SEQ ID NO: 167 - DNA, 24 bp, Artificial
  Primer
gaagttgactgtgcatggaaatcc

SEQ ID NO: 168 - DNA, 20 bp, Artificial
  Primer
ggcaggcttcacattttgga

SEQ ID NO: 169 - DNA, 21 bp, Artificial
  Primer
acacatacacacacacgcaca

SEQ ID NO: 170 - DNA, 20 bp, Artificial
  Primer
tggctgctcaaaaaatagca

SEQ ID NO: 171 - DNA, 22 bp, Artificial
  Primer
tttgaaggagtaagcactctgt

SEQ ID NO: 172 - DNA, 21 bp, Artificial
  Primer
cagacacaactgaagcaactc

SEQ ID NO: 173 - DNA, 22 bp, Artificial
  Primer
cacttagccacctgaaatagat

SEQ ID NO: 174 - DNA, 20 bp, Artificial
  Primer
agcaactgccacttcacttc

SEQ ID NO: 175 - DNA, 19 bp, Artificial
  Primer
tttggacttgccaaacctc

SEQ ID NO: 176 - DNA, 23 bp, Artificial
  Primer
tcagagcaacagaactaataaga

SEQ ID NO: 177 - DNA, 22 bp, Artificial
  Primer
gaacagtggttacctgtctgtc

SEQ ID NO: 178 - DNA, 21 bp, Artificial
  Primer
ctgcctaaccttcctggcgtc

SEQ ID NO: 179 - DNA, 21 bp, Artificial
  Primer
ttctactctccagcctcctcc

SEQ ID NO: 180 - DNA, 20 bp, Artificial
  Primer
gttggctccaagagcaagtc

SEQ ID NO: 181 - DNA, 25 bp, Artificial
  Primer
caggccttcaccaacagttcactga

SEQ ID NO: 182 - DNA, 25 bp, Artificial
  Primer
gattccacagtgccagacccaagcc

SEQ ID NO: 183 - DNA, 22 bp, Artificial
  Primer
tcctacttttgcattccagtcc

SEQ ID NO: 184 - DNA, 22 bp, Artificial
  Primer
accactaaagtcaaagaagccg

SEQ ID NO: 185 - DNA, 20 bp, Artificial
  Primer
actatggacatttggggcag

SEQ ID NO: 186 - DNA, 22 bp, Artificial
  Primer
agtaggtggagatcaaggatgc

SEQ ID NO: 187 - DNA, 25 bp, Artificial
  Primer
gtttgatactatgtccctttgtgtg

SEQ ID NO: 188 - DNA, 20 bp, Artificial
  Primer
gagtagctgcccctgttctc

SEQ ID NO: 189 - DNA, 23 bp, Artificial
  Primer
tttgagaacttttgtttctgagc

SEQ ID NO: 190 - DNA, 24 bp, Artificial
  Primer
ttattttgcttttctgattttgtg

SEQ ID NO: 191 - DNA, 20 bp, Artificial
  Primer
ccaacgggttaaaagcactg

SEQ ID NO: 192 - DNA, 21 bp, Artificial
  Primer
ttcctgttccttcctcatctg